SEQUENCE LISTING (90 sequences)

SEQ ID NO: 1 - protein, 10 aa, synthetic construct
GFTFSTYYIH

SEQ ID NO: 2 - protein, 17 aa, synthetic construct
WIHPSGKYTYYADSVKG

SEQ ID NO: 3 - protein, 9 aa, synthetic construct
HYQVGAMDY

SEQ ID NO: 4 - protein, 11 aa, synthetic construct
RASQSVSSAVA

SEQ ID NO: 5 - protein, 7 aa, synthetic construct
SASSLYS

SEQ ID NO: 6 - protein, 9 aa, synthetic construct
QQSGRSLYT

SEQ ID NO: 7 - protein, 8 aa, synthetic construct
GFTFSTYY

SEQ ID NO: 8 - protein, 8 aa, synthetic construct
IHPSGKYT

SEQ ID NO: 9 - protein, 11 aa, synthetic construct
ARHYQVGAMDY

SEQ ID NO: 10 - protein, 6 aa, synthetic construct
QSVSSA

SEQ ID NO: 11 - skipped

SEQ ID NO: 12 - protein, 5 aa, synthetic construct
TYYIH

SEQ ID NO: 13 - protein, 7 aa, synthetic construct
GFTFSTY

SEQ ID NO: 14 - protein, 4 aa, synthetic construct
PSGK

SEQ ID NO: 15 - protein, 7 aa, synthetic construct
YQVGAMD

SEQ ID NO: 16 - protein, 7 aa, synthetic construct
SQSVSSA

SEQ ID NO: 17 - protein, 6 aa, synthetic construct
SGRSLY

SEQ ID NO: 18 - protein, 6 aa, synthetic construct
STYYIH

SEQ ID NO: 19 - protein, 13 aa, synthetic construct
WVAWIHPSGKYTY

SEQ ID NO: 20 - protein, 10 aa, synthetic construct
ARHYQVGAMD

SEQ ID NO: 21 - protein, 7 aa, synthetic construct
SSAVAWY

SEQ ID NO: 22 - protein, 10 aa, synthetic construct
LLIYSASSLY

SEQ ID NO: 23 - protein, 8 aa, synthetic construct
QQSGRSLY

SEQ ID NO: 24 - protein, 10 aa, synthetic construct
WIHPSGKYTY

SEQ ID NO: 25 - protein, 118 aa, synthetic construct
EVQLVESGGGLVQPGGSLRLSCAASGFTFSTYYIHWVRQAPGKGLEWVAWIHPSGKYTYYADSVKGRFTISADTSKNTAYLQMNSLRAEDTAVYYCARHYQVGAMDYWGQGTLVTVSS

SEQ ID NO: 26 - protein, 107 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQSGRSLYTFGQGTKVEIK

SEQ ID NO: 27 - protein, 10 aa, synthetic construct
GSSFTGYNMN

SEQ ID NO: 28 - protein, 17 aa, synthetic construct
AIDPYYGGTYYADSVKG

SEQ ID NO: 29 - protein, 4 aa, synthetic construct
GMEY

SEQ ID NO: 30 - protein, 16 aa, synthetic construct
RSSQSLVHRNGNTYLH

SEQ ID NO: 31 - protein, 7 aa, synthetic construct
KVSNRFS

SEQ ID NO: 32 - protein, 10 aa, synthetic construct
SQSTHVPPLT

SEQ ID NO: 33 - protein, 8 aa, synthetic construct
GSSFTGYN

SEQ ID NO: 34 - protein, 8 aa, synthetic construct
IDPYYGGT

SEQ ID NO: 35 - protein, 6 aa, synthetic construct
ASGMEY

SEQ ID NO: 36 - protein, 11 aa, synthetic construct
QSLVHRNGNTY

SEQ ID NO: 37 - skipped

SEQ ID NO: 38 - protein, 5 aa, synthetic construct
GYNMN

SEQ ID NO: 39 - protein, 7 aa, synthetic construct
GSSFTGY

SEQ ID NO: 40 - protein, 4 aa, synthetic construct
PYYG

SEQ ID NO: 41 - skipped

SEQ ID NO: 42 - protein, 12 aa, synthetic construct
SQSLVHRNGNTY

SEQ ID NO: 43 - protein, 7 aa, synthetic construct
STHVPPL

SEQ ID NO: 44 - protein, 6 aa, synthetic construct
TGYNMN

SEQ ID NO: 45 - protein, 13 aa, synthetic construct
WVAAIDPYYGGTY

SEQ ID NO: 46 - protein, 5 aa, synthetic construct
ASGME

SEQ ID NO: 47 - protein, 12 aa, synthetic construct
VHRNGNTYLHWY

SEQ ID NO: 48 - protein, 10 aa, synthetic construct
LLIHKVSNRF

SEQ ID NO: 49 - protein, 9 aa, synthetic construct
SQSTHVPPL

SEQ ID NO: 50 - protein, 10 aa, synthetic construct
AIDPYYGGTY

SEQ ID NO: 51 - protein, 113 aa, synthetic construct
EVQLVESGGGLVQPGGSLRLSCAASGSSFTGYNMNWVRQAPGKGLEWVAAIDPYYGGTYYADSVKGRFTISADTSKNTAYLQMNSLRAEDTAVYYCASGMEYWGQGTLVTVSS

SEQ ID NO: 52 - protein, 113 aa, synthetic construct
DIQMTQSPSSLSASVGDRVTITCRSSQSLVHRNGNTYLHWYQQKPGKAPKLLIHKVSNRFSGVPSRFSGSGSGTDFTLTISSLQPEDFATYYCSQSTHVPPLTFGQGTKVEIK

SEQ ID NO: 53 - protein, 441 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQSGRSLYTFGQGTKVEIKGQPREPQVCTLPPSRDELTKNQVSLTCLVRGFYPSDIAVEWESNGQPENNYKTTPPVLDSDGSFFLYSKLTVDKSRWQQGNVFSCSVMHEALHNHYTQKSLSLSPGKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 225 aa, synthetic construct
EVQLVESGGGLVQPGGSLRLSCAASGFTFSTYYIHWVRQAPGKGLEWVAWIHPSGKYTYYADSVKGRFTISADTSKNTAYLQMNSLRAEDTAVYYCARHYQVGAMDYWGQGTLVTVSSAQPREPQVYTLPPCRDWLTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - protein, 447 aa, synthetic construct
DIQMTQSPSSLSASVGDRVTITCRSSQSLVHRNGNTYLHWYQQKPGKAPKLLIHKVSNRFSGVPSRFSGSGSGTDFTLTISSLQPEDFATYYCSQSTHVPPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 56 - protein, 216 aa, synthetic construct
EVQLVESGGGLVQPGGSLRLSCAASGSSFTGYNMNWVRQAPGKGLEWVAAIDPYYGGTYYADSVKGRFTISADTSKNTAYLQMNSLRAEDTAVYYCASGMEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 57 - protein, 447 aa, synthetic construct
DIQMTQSPSSLSASVGDRVTITCRSSQSLVHRNGNTYLHWYQQKPGKAPKLLIHKVSNRFSGVPSRFSGSGSGTDFTLTISSLQPEDFATYYCSQSTHVPPLTFGQGTKVEIKGQPREPQVCTLPPSRDELTKNQVSLTCLVRGFYPSDIAVEWESNGQPENNYKTTPPVLDSDGSFFLYSKLTVDKSRWQQGNVFSCSVMHEALHNHYTQKSLSLSPGKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - protein, 220 aa, synthetic construct
EVQLVESGGGLVQPGGSLRLSCAASGSSFTGYNMNWVRQAPGKGLEWVAAIDPYYGGTYYADSVKGRFTISADTSKNTAYLQMNSLRAEDTAVYYCASGMEYWGQGTLVTVSSAQPREPQVYTLPPCRDWLTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - protein, 441 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQSGRSLYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 60 - protein, 221 aa, synthetic construct
EVQLVESGGGLVQPGGSLRLSCAASGFTFSTYYIHWVRQAPGKGLEWVAWIHPSGKYTYYADSVKGRFTISADTSKNTAYLQMNSLRAEDTAVYYCARHYQVGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 61 - protein, 441 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQSGRSLYTFGQGTKVEIKGQPREPQVCTLPPSRDELTKNQVSLTCLVRGFYPSDIAVEWESNGQPENNYKTTPPVLDSDGSFFLYSKLTVDKSRWQQGNVFSCSVMHEALHNHYTQKSLSLSPGKDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALKAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 225 aa, synthetic construct; identical to SEQ ID NO: 54

SEQ ID NO: 63 - protein, 447 aa, synthetic construct
DIQMTQSPSSLSASVGDRVTITCRSSQSLVHRNGNTYLHWYQQKPGKAPKLLIHKVSNRFSGVPSRFSGSGSGTDFTLTISSLQPEDFATYYCSQSTHVPPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALKAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 64 - protein, 216 aa, synthetic construct; identical to SEQ ID NO: 56

SEQ ID NO: 65 - protein, 447 aa, synthetic construct
DIQMTQSPSSLSASVGDRVTITCRSSQSLVHRNGNTYLHWYQQKPGKAPKLLIHKVSNRFSGVPSRFSGSGSGTDFTLTISSLQPEDFATYYCSQSTHVPPLTFGQGTKVEIKGQPREPQVCTLPPSRDELTKNQVSLTCLVRGFYPSDIAVEWESNGQPENNYKTTPPVLDSDGSFFLYSKLTVDKSRWQQGNVFSCSVMHEALHNHYTQKSLSLSPGKDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALKAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 66 - protein, 220 aa, synthetic construct; identical to SEQ ID NO: 58

SEQ ID NO: 67 - protein, 441 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQSGRSLYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALKAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 68 - protein, 221 aa, synthetic construct; identical to SEQ ID NO: 60

SEQ ID NO: 69 - protein, 334 aa, synthetic construct
GQPREPQVCTLPPSRDELTKNQVSLTCLVRGFYPSDIAVEWESNGQPENNYKTTPPVLDSDGSFFLYSKLTVDKSRWQQGNVFSCSVMHEALHNHYTQKSLSLSPGKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - protein, 107 aa, synthetic construct
AQPREPQVYTLPPCRDWLTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 334 aa, synthetic construct
RTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 72 - protein, 103 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 73 - protein, 107 aa, synthetic construct
GQPREPQVCTLPPSRDELTKNQVSLTCLVRGFYPSDIAVEWESNGQPENNYKTTPPVLDSDGSFFLYSKLTVDKSRWQQGNVFSCSVMHEALHNHYTQKSLSLSPGK

SEQ ID NO: 74 - protein, 110 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 75 - protein, 107 aa, synthetic construct
GQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - protein, 107 aa, synthetic construct [sequence omitted]

SEQ ID NO: 77 - protein, 110 aa, synthetic construct; identical to SEQ ID NO: 74

SEQ ID NO: 78 - protein, 107 aa, synthetic construct
GQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 79 - protein, 534 aa, Homo sapiens
MLRRRGSPGMGVHVGAALGALWFCLTGALEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYQGYPEAEVFWQDGQGVPLTGNVTTSQMANEQGLFDVHSILRVVLGANGTYSCLVRNPVLQQDAHSSVTITPQRSPTGAVEVQVPEDPVVALVGTDATLRCSFSPEPGFSLAQLNLIWQLTDTKQLVHSFTEGRDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEALWVTVGLSVCLIALLVALAFVCWRKIKQSCEEENAGAEDQDGEGEGSKTALQPLKHSDSKEDDGQEIA

SEQ ID NO: 80 - protein, 533 aa, Homo sapiens
MWLGRRALCALVLLLACASLGLLYASTRDAPGLRLPLAPWAPPQSPRRPELPDLAPEPRYAHIPVRIKEQVVGLLAWNNCSCESSGGGLPLPFQKQVRAIDLTKAFDPAELRAASATREQEFQAFLSRSQSPADQLLIAPANSPLQYPLQGVEVQPLRSILVPGLSLQAASGQEVYQVNLTASLGTWDVAGEVTGVTLTGEGQADLTLVSPGLDQLNRQLQLVTYSSRSYQTNTADTVRFSTEGHEAAFTIRIRHPPNPRLYPPGSLPQGAQYNISALVTIATKTFLRYDRLRALITSIRRFYPTVTVVIADDSDKPERVSGPYVEHYLMPFGKGWFAGRNLAVSQVTTKYVLWVDDDFVFTARTRLERLVDVLERTPLDLVGGAVREISGFATTYRQLLSVEPGAPGLGNCLRQRRGFHHELVGFPGCVVTDGVVNFFLARTDKVREVGFDPRLSRVAHLEFFLDGLGSLRVGSCSDVVVDHASKLKLPWTSRDAGAETYARYRYPGSLDESQMAKHRLLFFKHRLQCMTSQ

SEQ ID NO: 81 - protein, 222 aa, synthetic construct
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 82 - protein, 222 aa, synthetic construct
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 222 aa, synthetic construct
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 84 - protein, 222 aa, synthetic construct
CPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALKAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - protein, 222 aa, synthetic construct
CPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALKAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 86 - protein, 222 aa, synthetic construct
CPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALKAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 87 - protein, 110 aa, synthetic construct
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALKAPIEKTISKAK

SEQ ID NO: 88 - protein, 29 aa, synthetic construct
GPASSAVPLSPAHPPHGSMCWSHWFSRGL

SEQ ID NO: 89 - protein, 6 aa, synthetic construct
GKDTWA

SEQ ID NO: 90 - protein, 20 aa, synthetic construct
GARPGWRDGQTVGGPRKRLV

(19) United States
(12) Patent Application Publication
(10) Pub. No.: US 2025/0353910 A1
(43) Pub. Date: Nov. 20, 2025

(54) MULTISPECIFIC BINDING AGENTS THAT TARGET B7H3 AND GD2 AND USES THEREOF

(71) Applicant: Invenra Inc., Madison, WI (US)

(72) Inventors: Jonathan Harry DAVIS, Madison, WI (US); Nicholas Michael MARSHALL, Middleton, WI (US)

(21) Appl. No.: 18/980,705

(22) Filed: Dec. 13, 2024

Related U.S. Application Data
(63) Continuation of application No. PCT/US2023/068384, filed on Jun. 13, 2023.
(60) Provisional application No. 63/352,208, filed on Jun. 14, 2022.

(51) Int. Cl. C07K 16/28; A61K 39/00; A61P 35/00; C07K 16/30

(52) U.S. Cl. CPC C07K 16/2827; A61P 35/00; C07K 16/3084; A61K 2039/505; C07K 2317/31; C07K 2317/33; C07K 2317/35; C07K 2317/732; C07K 2317/92; C07K 2317/94

(57) ABSTRACT

The present disclosure provides multispecific binding agents (e.g., antibodies, such as bispecific antibodies) that have a first binding domain that binds to B7H3, including human B7H3, and a second binding domain that binds to GD2, such as human GD2, and uses thereof.

### CROSS-REFERENCE TO RELATED APPLICATION

This application is a continuation of International Patent Application No. PCT/US2023/068384, filed Jun. 13, 2023, which claims the benefit of priority to U.S. provisional Application No. 63/352,208, filed Jun. 14, 2022, the entire contents of each of which are herein incorporated by reference.

### INCORPORATION BY REFERENCE OF SEQUENCE LISTING

The instant application contains a Sequence Listing, which has been submitted electronically. The Sequence Listing titled 209070-011003US_SL.xml, which was created on Dec. 9, 2024 and is 86,457 bytes in size, is herein incorporated by reference in its entirety.

### FIELD

The present disclosure relates generally to multispecific binding agents, such as bispecific antibodies, that have a first binding domain that binds to B7H3, including human B7H3, and a second binding domain that binds to GD2, and methods of their use.

### BACKGROUND

With the current biotherapeutic market dominated by antibody molecules, bispecific antibodies represent a key component of the next-generation of antibody therapy. Bispecific antibodies can target two different antigens at the same time, such as simultaneously binding tumor cell receptors and recruiting cytotoxic immune cells. Structural diversity has been fast-growing in the bispecific antibody field, creating a plethora of novel bispecific antibody scaffolds, which provide great functional variety. Two common formats of bispecific antibodies on the market are the single-chain variable fragment (scFv)-based (with or without an Fc fragment) antibody and the full-length IgG-like asymmetric antibody. Unlike the conventional monoclonal antibodies, great production challenges with respect to the quantity, quality, and stability of bispecific antibodies have hampered their wider clinical application and acceptance.

There are mainly two problems that must be solved to produce the desired IgG-like bispecific antibody—the heterodimerization of two different heavy chains and the discrimination between preferred and unwanted light-chain/heavy-chain interactions. A multiplicity of judicious genetic and cellular engineering strategies, including but not limited to, quadroma technology, knobs-into-holes, common heavy chain, and common light-chain strategies, CrossMab, orthogonal mutations in the CH1/CL interfaces, and co-culture methods, have been implemented in the attempt to produce optimized Y-shape IgG-like bispecific antibodies.

Bispecific antibody heavy-chain heterodimerization, especially within the CH3 region, represents an approach involving multiple design strategies, including but not limited to, steric mutation, electrostatic steering interactions and charge difference of heavy chains to facilitate purification. A multiplicity of approaches are often applied together in attempts to achieve bispecific antibody heavy-chain heterodimerization with minimal homodimer formation.

Mammalian cells are the predominant workhorses for IgG production in industry, and various production platforms are widely scalable for high titers (e.g., multiple grams per liter of culture) of conventional monoclonal antibodies in order to meet clinical and commercial demands. However, the production of bispecific antibodies is much more complex. One level of complexity involves the strategy and design of plasmids for expression of bispecific antibodies. Single plasmid systems using four open reading frames can be challenging to create and are often unable to produce diverse ratios of chain expression, resulting in the requirement for at least two and as many as four plasmids to express two heterodimerized heavy chains and either a common light chain or two different light-chains. Recommendations have been made to express heavy chain and light chain on separate plasmids because the manipulation of the plasmid ratio may be an easier and/or efficient approach to optimize protein assembly for desired products. Subsequently, a laborious and time-consuming process is typically needed to select the most desirable clonal cell lines from a heterogeneous stable transfectant pool for large-scale antibody production. CHO cells are well-known for high protein productivity, low contamination rates, and human immunological compatibility. The expression levels for conventional monoclonal antibodies via stable CHO cells can reach >3 g/L and sometimes >5 g/L and beyond and be successfully scaled up in bioreactors to large volumes. Nevertheless, the yield of bispecific IgG-like antibody from CHO cells has been significantly lower. A key challenge in addition to the challenge of low yield of bispecific antibody is how to produce uniform bispecific antibody with high quality and limited or negligible side products and impurities.

Although a variety of bispecific antibodies have been reported, including bispecific antibodies that target B7H3 and GD2 (e.g., WO2021/168379), the challenges of producing bispecific antibodies of high quality and high yield, including B7H3×GD2 bispecific antibodies, remain. The multispecific binding agents, compositions and methods provide herein satisfy this need and provide related advantages.

### SUMMARY

In some aspects, provided herein are composition that include multispecific binding agents (e.g., antibodies, such as bispecific antibodies) that bind to B7H3 and GD2. Such compositions are useful in methods of treating, preventing, or alleviating diseases, including cancer, and one or more symptoms associated with the diseases. The compositions and methods provided herein can target B7H3 and GD2 co-expressing cells. In particular, the multispecific binding agents (e.g., antibodies, such as bispecific antibodies) that bind to B7H3 and GD2 can selectively bind to B7H3 and GD2 co-expressing cells, including cancer cells.

In some aspects, provided herein is a bispecific antibody comprising a first polypeptide chain, a second polypeptide chain, a third polypeptide chain, and a fourth polypeptide chain, wherein the first polypeptide chain and the second polypeptide chain comprise an antigen binding domain for B7 Homolog 3 (B7H3), wherein the third polypeptide chain and the fourth polypeptide chain comprise an antigen binding domain for disialoganglioside (GD2), and wherein: the first polypeptide chain comprises the amino acid sequence of SEQ ID NO: 53; the second polypeptide chain comprises the amino acid sequence of SEQ ID NO: 54; the third polypeptide chain comprises the amino acid sequence of SEQ ID NO: 55; the fourth polypeptide chain comprises the amino acid sequence of SEQ ID NO: 56.

In some aspects, provided herein is a bispecific antibody comprising a first polypeptide chain, a second polypeptide chain, a third polypeptide chain, and a fourth polypeptide chain, wherein the first polypeptide chain and the second polypeptide chain comprise an antigen binding domain for disialoganglioside (GD2), wherein the third polypeptide chain and the fourth polypeptide chain comprise an antigen binding domain for B7 Homolog 3 (B7H3), and wherein: the first polypeptide chain comprises the amino acid sequence of SEQ ID NO: 57; the second polypeptide chain comprises the amino acid sequence of SEQ ID NO: 58; the third polypeptide chain comprises the amino acid sequence of SEQ ID NO: 59; the fourth polypeptide chain comprises the amino acid sequence of SEQ ID NO: 60.

In some aspects, provided herein is a bispecific antibody comprising a first polypeptide chain, a second polypeptide chain, a third polypeptide chain, and a fourth polypeptide chain, wherein the first polypeptide chain and the second polypeptide chain comprise an antigen binding domain for B7 Homolog 3 (B7H3), wherein the third polypeptide chain and the fourth polypeptide chain comprise an antigen binding domain for disialoganglioside (GD2), and wherein: the first polypeptide chain comprises a light chain variable (VL) region and constant regions, wherein the VL region comprises a VL CDR1, a VL CDR2, and a VL CDR3 amino acid sequence as set forth in SEQ ID NO: 26 and the constant regions comprise the amino acid sequence of SEQ ID NO: 69; the second polypeptide chain comprises a heavy chain variable (VH) region and a constant region, wherein the VH region comprises a VH CDR1, a VH CDR2, and a VH CDR3 amino acid sequence as set forth in SEQ ID NO: 25 and the constant region comprises the amino acid sequence of SEQ ID NO: 70; the third polypeptide chain comprises a light chain variable (VL) region and constant regions, wherein the VL region comprises a VL CDR1, a VL CDR2, and a VL CDR3 amino acid sequence as set forth in SEQ ID NO: 52 and the constant regions comprise the amino acid sequence of SEQ ID NO: 71; the fourth polypeptide chain comprises a heavy chain variable (VH) region and a constant region, wherein the VH region comprises a VH CDR1, a VH CDR2, and a VH CDR3 amino acid sequence as set forth in SEQ ID NO: 51 and the constant region comprises the amino acid sequence of SEQ ID NO: 72. In some embodiments, the VL CDR1 of the first polypeptide chain comprises the amino acid sequence of SEQ ID NO: 4; the VL CDR2 of the first polypeptide chain comprises the amino acid sequence of SEQ ID NO: 5; the VL CDR3 of the first polypeptide chain comprises the amino acid sequence of SEQ ID NO: 6; the VH CDR1 of the second polypeptide chain comprises the amino acid sequence of SEQ ID NO: 1; the VH CDR2 of the second polypeptide chain comprises the amino acid sequence of SEQ ID NO: 2; the VH CDR3 of the second polypeptide chain comprises the amino acid sequence of SEQ ID NO: 3; the VL CDR1 of the third polypeptide chain comprises the amino acid sequence of SEQ ID NO: 30; the VL CDR2 of the third polypeptide chain comprises the amino acid sequence of SEQ ID NO: 31; the VL CDR3 of the third polypeptide chain comprises the amino acid sequence of SEQ ID NO: 32; the VH CDR1 of the fourth polypeptide chain comprises the amino acid sequence of SEQ ID NO: 27; the VH CDR2 of the fourth polypeptide chain comprises the amino acid sequence of SEQ ID NO: 28; and the VH CDR3 of the fourth polypeptide chain comprises the amino acid sequence of SEQ ID NO: 29. In some embodiments, the VL CDR1 of the first polypeptide chain comprises the amino acid sequence of SEQ ID NO: 10; the VL CDR2 of the first polypeptide chain comprises the amino acid sequence of SEQ ID NO: 11; the VL CDR3 of the first polypeptide chain comprises the amino acid sequence of SEQ ID NO: 6; the VH CDR1 of the second polypeptide chain comprises the amino acid sequence of SEQ ID NO: 7; the VH CDR2 of the second polypeptide chain comprises the amino acid sequence of SEQ ID NO: 8; the VH CDR3 of the second polypeptide chain comprises the amino acid sequence of SEQ ID NO: 9; the VL CDR1 of the third polypeptide chain comprises the amino acid sequence of SEQ ID NO: 36; the VL CDR2 of the third polypeptide chain comprises the amino acid sequence of SEQ ID NO: 37; the VL CDR3 of the third polypeptide chain comprises the amino acid sequence of SEQ ID NO: 32; the VH CDR1 of the fourth polypeptide chain comprises the amino acid sequence of SEQ ID NO: 33; the VH CDR2 of the fourth polypeptide chain comprises the amino acid sequence of SEQ ID NO: 34; and the VH CDR3 of the fourth polypeptide chain comprises the amino acid sequence of SEQ ID NO: 35. In some embodiments, the VL CDR1 of the first polypeptide chain comprises the amino acid sequence of SEQ ID NO: 4; the VL CDR2 of the first polypeptide chain comprises the amino acid sequence of SEQ ID NO: 5; the VL CDR3 of the first polypeptide chain comprises the amino acid sequence of SEQ ID NO: 6; the VH CDR1 of the second polypeptide chain comprises the amino acid sequence of SEQ ID NO: 12; the VH CDR2 of the second polypeptide chain comprises the amino acid sequence of SEQ ID NO: 2; the VH CDR3 of the second polypeptide chain comprises the amino acid sequence of SEQ ID NO: 3; the VL CDR1 of the third polypeptide chain comprises the amino acid sequence of SEQ ID NO: 30; the VL CDR2 of the third polypeptide chain comprises the amino acid sequence of SEQ ID NO: 31; the VL CDR3 of the third polypeptide chain comprises the amino acid sequence of SEQ ID NO: 32; the VH CDR1 of the fourth polypeptide chain comprises the amino acid sequence of SEQ ID NO: 38; the VH CDR2 of the fourth polypeptide chain comprises the amino acid sequence of SEQ ID NO: 28; and the VH CDR3 of the fourth polypeptide chain comprises the amino acid sequence of SEQ ID NO: 29.

In some aspects, provided herein is a bispecific antibody comprising a first polypeptide chain, a second polypeptide chain, a third polypeptide chain, and a fourth polypeptide chain, wherein the first polypeptide chain and the second polypeptide chain comprise an antigen binding domain for disialoganglioside (GD2), wherein the third polypeptide chain and the fourth polypeptide chain comprise an antigen binding domain for B7 Homolog 3 (B7H3), and wherein: the first polypeptide chain comprises a light chain variable (VL) region and constant regions, wherein the VL region comprises a VL CDR1, a VL CDR2, and a VL CDR3 amino acid sequence as set forth in SEQ ID NO: 52 and the constant regions comprise the amino acid sequence of SEQ ID NO: 69; the second polypeptide chain comprises a heavy chain variable (VH) region and a constant region, wherein the VH region comprises a VH CDR1, a VH CDR2, and a VH CDR3 amino acid sequence as set forth in SEQ ID NO: 51 and the constant region comprises the amino acid sequence of SEQ ID NO: 70; the third polypeptide chain comprises a light chain variable (VL) region and constant regions, wherein the VL region comprises a VL CDR1, a VL CDR2, and a VL CDR3 amino acid sequence as set forth in SEQ ID NO: 26 and the constant regions comprise the amino acid sequence of SEQ ID NO: 71; the fourth polypeptide chain comprises a heavy chain variable (VH) region and a constant region, wherein the VH region comprises a VH CDR1, a VH CDR2, and a VH CDR3 amino acid sequence as set forth in SEQ ID NO: 25 and the constant region comprises the amino acid sequence of SEQ ID NO: 72. In some embodiments, a VL CDR1 of the first polypeptide chain comprises the amino acid sequence of SEQ ID NO: 30; a VL CDR2 of the first polypeptide chain comprises the amino acid sequence of SEQ ID NO: 31; a VL CDR3 of the first polypeptide chain comprises the amino acid sequence of SEQ ID NO: 32; a VH CDR1 of the second polypeptide chain comprises the amino acid sequence of SEQ ID NO: 27; a VH CDR2 of the second polypeptide chain comprises the amino acid sequence of SEQ ID NO: 28; a VH CDR3 of the second polypeptide chain comprises the amino acid sequence of SEQ ID NO: 29; a VL CDR1 of the third polypeptide chain comprises the amino acid sequence of SEQ ID NO: 4; a VL CDR2 of the third polypeptide chain comprises the amino acid sequence of SEQ ID NO: 5; a VL CDR3 of the third polypeptide chain comprises the amino acid sequence of SEQ ID NO: 6; a VH CDR1 of the fourth polypeptide chain comprises the amino acid sequence of SEQ ID NO: 1; a VH CDR2 of the fourth polypeptide chain comprises the amino acid sequence of SEQ ID NO: 2; and a VH CDR3 of the fourth polypeptide chain comprises the amino acid sequence of SEQ ID NO: 3. In some embodiments, the a VL CDR1 of the first polypeptide chain comprises the amino acid sequence of SEQ ID NO: 36; a VL CDR2 of the first polypeptide chain comprises the amino acid sequence of SEQ ID NO: 37; a VL CDR3 of the first polypeptide chain comprises the amino acid sequence of SEQ ID NO: 32; a VH CDR1 of the second polypeptide chain comprises the amino acid sequence of SEQ ID NO: 33; a VH CDR2 of the second polypeptide chain comprises the amino acid sequence of SEQ ID NO: 34; a VH CDR3 of the second polypeptide chain comprises the amino acid sequence of SEQ ID NO: 35; a VL CDR1 of the third polypeptide chain comprises the amino acid sequence of SEQ ID NO: 10; a VL CDR2 of the third polypeptide chain comprises the amino acid sequence of SEQ ID NO: 11; a VL CDR3 of the third polypeptide chain comprises the amino acid sequence of SEQ ID NO: 6; a VH CDR1 of the fourth polypeptide chain comprises the amino acid sequence of SEQ ID NO: 7; a VH CDR2 of the fourth polypeptide chain comprises the amino acid sequence of SEQ ID NO: 8; and a VH CDR3 of the fourth polypeptide chain comprises the amino acid sequence of SEQ ID NO: 9. In some embodiments, the a VL CDR1 of the first polypeptide chain comprises the amino acid sequence of SEQ ID NO: 30; a VL CDR2 of the first polypeptide chain comprises the amino acid sequence of SEQ ID NO: 31; a VL CDR3 of the first polypeptide chain comprises the amino acid sequence of SEQ ID NO: 32; a VH CDR1 of the second polypeptide chain comprises the amino acid sequence of SEQ ID NO: 38; a VH CDR2 of the second polypeptide chain comprises the amino acid sequence of SEQ ID NO: 28; a VH CDR3 of the second polypeptide chain comprises the amino acid sequence of SEQ ID NO: 29; a VL CDR1 of the third polypeptide chain comprises the amino acid sequence of SEQ ID NO: 4; a VL CDR2 of the third polypeptide chain comprises the amino acid sequence of SEQ ID NO: 5; a VL CDR3 of the third polypeptide chain comprises the amino acid sequence of SEQ ID NO: 6; a VH CDR1 of the fourth polypeptide chain comprises the amino acid sequence of SEQ ID NO: 12; a VH CDR2 of the fourth polypeptide chain comprises the amino acid sequence of SEQ ID NO: 2; and a VH CDR3 of the fourth polypeptide chain comprises the amino acid sequence of SEQ ID NO: 3.

In some embodiments, the VH region or VL region of a bispecific antibody described herein further comprises human framework sequences. In some embodiments, the VH region and VL region of a bispecific antibody described herein further comprises human framework sequences. In some embodiments, the VH region or VL region further comprises a framework 1 (FR1), a framework 2 (FR2), a framework 3 (FR3) and/or a framework 4 (FR4) sequence. In some embodiments, the VH region and VL region further comprises a framework 1 (FR1), a framework 2 (FR2), a framework 3 (FR3) and a framework 4 (FR4) sequence.

In some embodiments, the bispecific antibody provided herein is a monoclonal antibody. In some embodiments, the monoclonal antibody is a humanized, human or chimeric antibody.

In some embodiments, the bispecific antibody provided herein is conjugated or recombinantly fused to a diagnostic agent, detectable agent or therapeutic agent. In some embodiments, the therapeutic agent is a chemotherapeutic agent, cytotoxin, or drug.

In some aspects, provided herein is one or more vectors comprising one or more polynucleotides encoding the first polypeptide chain, the second polypeptide chain, the third polypeptide chain, and the fourth polypeptide chain of bispecific antibody described herein. In some aspects, provided herein is a cell comprising the one or more vectors.

In some aspects, provided herein is a method of generating a bispecific antibody composition comprising culturing the cell provided herein in a culture medium to produce the bispecific antibody in the culture medium, isolating the bispecific antibody from the culture medium, and purifying the bispecific antibody isolated from the culture medium, thereby generating the bispecific antibody composition. In some embodiments, the bispecific antibody provided herein isolating comprises: contacting the culture medium with a protein A or anti-CH1 based resin; and eluting the bispecific antibody from the protein A or anti-CH1 based resin. In some embodiments, the bispecific antibody provided herein the purifying further comprises: contacting the eluted bispecific antibody with a cation exchange resin; and eluting the bispecific antibody from the cation exchange resin.

In some aspects, provided herein is a pharmaceutical composition comprising an effective amount of the bispecific antibody provided herein and a pharmaceutically acceptable carrier. In some embodiments, the effective amount of the bispecific antibody is effective to treat a cancer in a subject. In some embodiments, the cancer is selected from a brain cancer, an eye cancer, a lung cancer, a skin cancer, and a sarcoma. In some embodiments, the brain cancer is neuroblastoma or glioblastoma. In some embodiments, the eye cancer is retinoblastoma. In some embodiments, the lung cancer is small cell lung cancer. In some embodiments, the skin cancer is melanoma. In some embodiments, the sarcoma is selected from osteosarcoma, rhabdomyosarcoma, Ewing's sarcoma in children and adolescents, liposarcoma, fibrosarcoma, leiomyosarcoma and other soft tissue sarcomas in adults.

In some aspects, provided herein is a method of treating or preventing a cancer in a subject comprising administering to the subject the bispecific antibody of provided herein or the pharmaceutical composition provided herein.

In some aspects, provided herein is a method for alleviating one or more symptoms associated with a cancer in a subject comprising administering to the subject the bispecific antibody provided herein or the pharmaceutical composition provided herein. In some embodiments, the cancer is selected from a brain cancer, an eye cancer, a lung cancer, a skin cancer, and a sarcoma. In some embodiments, the brain cancer is neuroblastoma or glioblastoma. In some embodiments, the eye cancer is a retinoblastoma. In some embodiments, the lung cancer is small cell lung cancer. In some embodiments, the skin cancer is melanoma. In some embodiments, the sarcoma is selected from osteosarcoma, rhabdomyosarcoma, Ewing's sarcoma in children and adolescents, liposarcoma, fibrosarcoma, leiomyosarcoma and other soft tissue sarcomas in adults. In some embodiments, the method further comprises administering to the subject a radiotherapy.

In some aspects, provided herein is a method of selectively binding a cell co-expressing B7 Homolog 3 (B7H3) and disialoganglioside (GD2) in a subject comprising administering to the subject the bispecific antibody provided herein or the pharmaceutical composition provided herein.

### BRIEF DESCRIPTION OF THE DRAWINGS

FIG. 1 presents a schematic antibody construct, with respective naming conventions for the various domains in the first, second, third and fourth polypeptide chains, for the bispecific antibodies described herein. Domain A=VL; Domain B=variant CH3; Domain D=CH2; Domain E=variant CH3; Domain F=VH; Domain G=variant CH3; Domain H=VL; Domain I=CL—Kappa; Domain J=CH2; Domain K=variant CH3; Domain L=VH; Domain M=CH1.

FIG. 2A illustrates exemplary results from binding assays using B78 cells co-expressing human B7H3 and human GD2, further described in Example 2.

FIG. 2B illustrates exemplary results from binding assays using CHLA-20 cells, further described in Example 2.

FIG. 3A illustrate exemplary results from ADCC assays for Exemplary Bispecific Antibody 1 on B78 cells overexpressing both human B7H3 and human GD2 or B78 cells overexpressing human GD2, but not B7H3, further described in Example 3.

FIG. 3B illustrate exemplary results from ADCC assays multiple antibodies on B78 cells overexpressing both human B7H3 and human GD2, further described in Example 3.

FIG. 4 illustrate exemplary results from pharmacokinetic assays, further described in Example 4.

FIG. 5 illustrate exemplary results from immunogenicity assays, further described in Example 5.

FIG. 6 presents a schematic antibody construct, with corresponding molecular weights and isoelectric points (pl), for a properly formed exemplary bispecific antibody (Ex. bsAb. 1) having polypeptide chains 1, 2, 3 and 4, and improperly formed complexes of various polypeptide chains, the formation of which is described and assess in Example 6.

FIG. 7 illustrate exemplary results from bispecific antibody culture, isolation and purification using pooled antibody lots, further described in Example 6.

FIG. 8 illustrate exemplary results from bispecific antibody culture, isolation and purification using single clonal lots, further described in Example 6.

FIG. 9 illustrate exemplary results from SEC chromatography for protein A purified exemplary bispecific antibody, further described in Example 6.

FIG. 10 illustrate exemplary results from SEC chromatography for protein A purified exemplary bispecific antibody polished with cation exchange, further described in Example 6.

FIG. 11 illustrate exemplary results from HIC chromatography for protein A purified exemplary bispecific antibody polished with cation exchange, further described in Example 6.

FIG. 12 illustrate exemplary results from SMAC chromatography for protein A purified exemplary bispecific antibody polished with cation exchange, further described in Example 6.

FIG. 13 illustrate exemplary results from mass spectroscopy analysis for protein A purified exemplary bispecific antibody, further described in Example 6.

FIG. 14 illustrate exemplary results from mass spectroscopy analysis for protein A purified exemplary bispecific antibody polished with cation exchange, further described in Example 6.

### DETAILED DESCRIPTION

The present disclosure provides multispecific binding agents (e.g., antibodies, such as bispecific antibodies) that bind to B7H3 and GD2. Such multispecific binding agents include antibodies (e.g., antibodies, such as bispecific antibodies) that bind to B7H3, including antibodies that bind to human B7H3, and GD2, including antibodies that bind to human GD2. Such multispecific binding agents are useful in compositions and in methods of treating, preventing, or alleviating cancer, including one or more symptoms associated with a cancer. Such a cancer that the compositions and methods described herein can be useful for include, but not limited to, any cancer wherein the cancer or tumor cells express or overexpress B7H3 and GD2. Such cancers include a brain cancer (e.g., neuroblastoma or glioblastoma), an eye cancer (e.g., retinoblastoma), a lung cancer (e.g., small cell lung cancer), a skin cancer (e.g., melanoma), a sarcoma (e.g., osteosarcoma, rhabdomyosarcoma, Ewing's sarcoma in children and adolescents, liposarcoma, fibrosarcoma, leiomyosarcoma and other soft tissue sarcomas in adults). Such B7H3 and GD2 co-expressing cells can be selectively targeted using the compositions and methods described herein. In particular, the multispecific binding agents (e.g., antibodies, such as bispecific antibodies) that bind to B7H3 and GD2 can selectively bind to B7H3 and GD2 co-expressing cancer or tumor cells over non-cancer or non-tumor cells expressing either B7H3 or GD2 (e.g., cells of the peripheral nervous system which are known for expressing GD2).

The term “B7H3,” “B7-H3,” “B7 Homolog 3,” “B7H3 polypeptide,” “B7H3 protein,” or similar term refers to a polypeptide (“polypeptide” and “protein” are used interchangeably herein) or any native B7H3 from any vertebrate source, including mammals such as primates (e.g., humans, cynomolgus monkey (cyno)), dogs, and rodents (e.g., mice and rats), unless otherwise indicated. B7H3, also known in the art as “4Ig-B7-H3,” “B7 homolog 3 protein,” “CD276 antigen,” “CD276,” or “CD276 protein”, is a protein that in humans is encoded by the CD276 gene. The CD276 gene is also referred to as the B7H3gene, or similar terms. B7H3 has Ig-like V-type 1 domain at positions 29-139, Ig-like C2-type 1 domain at positions 145-238, Ig-like V-type 2 domain at positions 243-357, and Ig-like C2-type 2 domain at positions 363-456. B7H3 is a type I transmembrane protein in the immunoglobulin superfamily. B7H3 is known in the art as an immune checkpoint member of the B7 family. B7-H3 has been shown to bind an unidentified counter-receptor on activated T cells to costimulate the proliferation of CD4+ or CD8+ T cells. B7-H3 has also been found to enhance the induction of primary cytotoxic T lymphocytes and stimulate IFN-gamma production. In normal tissues, B7-H3 protein has limited expression, playing an inhibitory role in adaptive immunity, suppressing T cell activation and proliferation. In malignant tissues, B7-H3 is overexpressed, serving as an immune checkpoint molecule that inhibits tumor antigen-specific immune responses, or other pro-tumorigenic functions. The term B7H3 encompasses “full-length,” unprocessed B7H3, as well as any form of B7H3 or any fragment thereof that results from processing in the cell, including the four known alternatively spliced isoforms of 4Ig-B7-H3, or human B7H3, that differ in the length of the intracellular tail. The term B7H3 also encompasses naturally occurring variants of B7H3, such as SNP variants, splice variants and allelic variants. The full-length amino acid sequence of human B7H3 is provided below:

(SEQ ID NO: 79)
MLRRRGSPGMGVHVGAALGALWFCLTGALEVQVPEDPVVALVGTDATLC
CSFSPEPGFSLAQLNLIWQLTDTKQLVHSFAEGQDQGSAYANRTALFPD
LLAQGNASLRLQRVRVADEGSFTCFVSIRDFGSAAVSLQVAAPYSKPSM
TLEPNKDLRPGDTVTITCSSYQGYPEAEVFWQDGQGVPLTGNVTTSQMA
NEQGLFDVHSILRVVLGANGTYSCLVRNPVLQQDAHSSVTITPQRSPTG
AVEVQVPEDPVVALVGTDATLRCSFSPEPGFSLAQLNLIWQLTDTKQLV
HSFTEGRDQGSAYANRTALFPDLLAQGNASLRLQRVRVADEGSFTCFVS
IRDFGSAAVSLQVAAPYSKPSMTLEPNKDLRPGDTVTITCSSYRGYPEA
EVFWQDGQGVPLTGNVTTSQMANEQGLFDVHSVLRVVLGANGTYSCLVR
NPVLQQDAHGSVTITGQPMTFPPEALWVTVGLSVCLIALLVALAFVCWR
KIKQSCEEENAGAEDQDGEGEGSKTALQPLKHSDSKEDDGQEIA.

The second isoform differs from the canonical sequence in that positions 159-376 are missing. The third isoform differs from the canonical sequence in those positions 465-493 read GPASSAVPLSPAHPPHGSMCWSHWFSRGL (SEQ ID NO: 88), and positions 494-534 are missing. The fourth isoform differs from the canonical sequence in that positions 528-534 read GKDTWA. (SEQ ID NO: 89).

Other related B7H3 polypeptides that are also encompassed by the term B7H3 include fragments, derivatives (e.g., substitution, deletion, truncations, and insertion variants), fusion polypeptides, and interspecies homologs that retain B7H3 activity and/or are sufficient to generate an anti-B7H3 immune response. As those skilled in the art will appreciate, a multispecific binding agent (e.g., an antibody, such as a bispecific antibody) described herein can bind to a B7H3 polypeptide, a B7H3 polypeptide fragment, a B7H3 antigen, and/or a B7H3 epitope. An epitope may be part of a larger B7H3 antigen, which may be part of a larger B7H3 polypeptide fragment, which, in turn, may be part of a larger B7H3 polypeptide. B7H3 may exist in a native or denatured form. B7H3 polypeptides described herein may be isolated from a variety of sources, such as from human tissue types or from another source, or prepared by recombinant or synthetic methods. A B7H3 polypeptide may comprise a polypeptide having the same amino acid sequence as a corresponding B7H3 polypeptide derived from nature. Orthologs to the B7H3 polypeptide are also well known in the art.

The term “GD2,” “disialoganglioside,” “GD2 polypeptide,” or similar terms refers to a polypeptide (“polypeptide” and “protein” are used interchangeably herein) or any native GD2 from any vertebrate source, including mammals such as primates (e.g., humans, cynomolgus monkey (cyno)), dogs, and rodents (e.g., mice and rats), unless otherwise indicated. GD2, also known as “beta-1,4-N-acetylgalactosaminyl transferase-1,” “(N-acetylneuraminyl)-galactosylglucosylceramide,” “GM2/GD2 synthase,” “GaINAc-T,” or “GALGT,” is a protein that in humans is encoded by the B4GALNT1 gene. The B4GALNT1 gene is also referred to as the GALGT gene, the SIAT2 gene, or similar terms. GD2 belongs to the glycosyltransferase 2 family. GD2 is known in the art as an enzyme involved in the biosynthesis of complex gangliosides (G), which are mono-(M), di-(D), and tri-(T) sialic acid-containing glycosphingolipids generated by sequential glycosylations. In particular, GD2 catalyzes the transfer of N-acetylgalactosamine into GM3, GD3, and globotriaosylceramide by a beta-1,4 linkage. GD2, a gangliosides, is a complex, acidic glycolipid expressed on the outer cell membrane. GD2 is biosynthesized from precursor gangliosides GD3/GM3 by β-1,4-N-acetylgalactosaminyltransferase (GD2 synthase). GD2 is highly expressed in neuroblastoma (NB) cells. In normal tissues, GD2 expression is largely limited to neurons, skin melanocytes, and peripheral pain fibers. The term GD2 encompasses “full-length,” GD2, as well as any form of GD2 or any fragment thereof that results from processing in the cell, including the four known alternatively spliced isoforms of GD2 that differ in the length of the intracellular tail. The term GD2 also encompasses naturally occurring variants of GD2, such as SNP variants, splice variants and allelic variants. The full-length amino acid sequence of human GD2 is provided below:

(SEQ ID NO: 80)
MWLGRRALCALVLLLACASLGLLYASTRDAPGLRLPLAPWAPPQSPRR
PELPDLAPEPRYAHIPVRIKEQVVGLLAWNNCSCESSGGGLPLPFQKQ
VRAIDLTKAFDPAELRAASATREQEFQAFLSRSQSPADQLLIAPANSP
LQYPLQGVEVQPLRSILVPGLSLQAASGQEVYQVNLTASLGTWDVAGE
VTGVTLTGEGQADLTLVSPGLDQLNRQLQLVTYSSRSYQTNTADTVRF
STEGHEAAFTIRIRHPPNPRLYPPGSLPQGAQYNISALVTIATKTFLR
YDRLRALITSIRRFYPTVTVVIADDSDKPERVSGPYVEHYLMPFGKGW
FAGRNLAVSQVTTKYVLWVDDDFVFTARTRLERLVDVLERTPLDLVGG
AVREISGFATTYRQLLSVEPGAPGLGNCLRQRRGFHHELVGFPGCVVT
DGVVNFFLARTDKVREVGFDPRLSRVAHLEFFLDGLGSLRVGSCSDVV
VDHASKLKLPWTSRDAGAETYARYRYPGSLDESQMAKHRLLFFKHRLQ
CMTSQ.

The second isoform differs from the canonical sequence in that positions 74-128 are missing. The third isoform differs from the canonical sequence in that positions 238-328 read GARPGWRDGQ . . . TVGGPRKRLV(SEQ ID NO: 90), and positions 329-533 are missing.

Other related GD2 polypeptides that are also encompassed by the term GD2 include fragments, derivatives (e.g., substitution, deletion, truncations, and insertion variants), fusion polypeptides, and interspecies homologs that retain GD2 activity and/or are sufficient to generate an anti-GD2 immune response. As those skilled in the art will appreciate, a multispecific binding agent (e.g., an antibody, such as a bispecific antibody) described herein can bind to a GD2 polypeptide, a GD2 polypeptide fragment, a GD2 antigen, and/or a GD2 epitope. An epitope may be part of a larger GD2 antigen, which may be part of a larger GD2 polypeptide fragment, which, in turn, may be part of a larger GD2 polypeptide. GD2 may exist in a native or denatured form. GD2 polypeptides described herein may be isolated from a variety of sources, such as from human tissue types or from another source, or prepared by recombinant or synthetic methods. A GD2 polypeptide may comprise a polypeptide having the same amino acid sequence as a corresponding GD2 polypeptide derived from nature. Orthologs to the GD2 polypeptide are also well known in the art.

As used herein, the term “binding agent” or a grammatical equivalent thereof refers to a molecule (e.g., an antibody, such as a bispecific antibody) with one or more antigen binding sites that binds an antigen. In some embodiments, a multispecific binding agent as described herein is an antibody, antibody fragment, or other peptide-based molecule that binds to B7H3, such as human B7H3.

The term “antibody,” “immunoglobulin,” or “Ig” is used interchangeably herein, and is used in the broadest sense and specifically covers, for example polyclonal antibodies, monoclonal antibodies (including agonist, antagonist, neutralizing antibodies, full length monoclonal antibodies), antibody compositions with polyepitopic or monoepitopic specificity, recombinantly produced antibodies, monospecific antibodies, multispecific antibodies (including bispecific antibodies), synthetic antibodies, chimeric antibodies, humanized antibodies, or human versions of antibodies having full length heavy and/or light chains. The present disclosure also includes antibody fragments (and/or polypeptides that comprise antibody fragments) that retain B7H3 binding characteristics. Non-limiting examples of antibody fragments include antigen-binding regions and/or effector regions of the antibody, e.g., Fab, Fab′, F(ab′)2, Fv, scFv, (scFv)₂, single chain antibody molecule, dual variable region antibody, single variable region antibody, linear antibody, V region, a multispecific antibody formed from antibody fragments, F(ab)₂, Fd, Fc, diabody, di-diabody, disulfide-linked Fvs (dsFv), single-domain antibody (e.g., nanobody) or other fragments (e.g., fragments consisting of the variable regions of the heavy and light chains that are non-covalently coupled). In general terms, a variable (V) region domain may be any suitable arrangement of immunoglobulin heavy (VH) and/or light (VL) chain variable domains. For example, the present disclosure also includes tetrameric antibodies comprising two heavy chain and two light chain molecules, an antibody light chain monomer, and an antibody heavy chain monomer. Thus, for example, the V region domain may be dimeric and contain VH-VH, VH-VL, or VL-VL dimers that bind B7H3. If desired, the VH and VL chains may be covalently coupled either directly or through a linker to form a single chain Fv (scFv). For ease of reference, scFv proteins are referred to herein as included in the category “antibody fragments.” Another form of an antibody fragment is a peptide comprising one or more complementarity determining regions (CDRs) of an antibody. CDRs (also termed “minimal recognition units” or “hypervariable region”) can be obtained by constructing polynucleotides that encode the CDR of interest. Such polynucleotides are prepared, for example, by using the polymerase chain reaction to synthesize the variable region using mRNA of antibody-producing cells as a template (see, for example, Larrick et al., Methods: A Companion to Methods in Enzymology, 2:106 (1991); Courtenay-Luck, “Genetic Manipulation of Monoclonal Antibodies,” in Monoclonal Antibodies Production, Engineering and Clinical Application, Ritter et al. (eds.), page 166, Cambridge University Press (1995); and Ward et al., “Genetic Manipulation and Expression of Antibodies,” in Monoclonal Antibodies: Principles and Applications, Birch et al., (eds.), page 137, Wiley-Liss, Inc. (1995)). Antibody fragments may be incorporated into single domain antibodies, maxibodies, minibodies, intrabodies, diabodies, triabodies, tetrabodies, variable domains of new antigen receptors (v-NAR), and bis-single chain Fv regions (see, e.g., Hollinger and Hudson, Nature Biotechnology, 23(9):1126-1136, 2005). The binding agent, in some embodiments, contains a light chain and/or a heavy chain constant region, such as one or more constant regions, including one or more IgG1, IgG2, IgG3 and/or IgG4 constant regions. In some embodiments, antibodies can include epitope-binding fragments of any of the above. The antibodies described herein can be of any class (e.g., IgG, IgE, IgM, IgD, and IgA) or any subclass (e.g., IgG1, IgG2, IgG3, IgG4, IgA1, and IgA2) of immunoglobulin molecule. Antibodies may be agonistic antibodies or antagonistic antibodies.

The term “monospecific” when used in reference to a binding agent (e.g., an antibody) as used herein denotes a binding agent that has one or more binding sites each of which bind to the same epitope of the same antigen.

The term “multispecific” when used in reference to a binding agent (e.g., an antibody, including a bispecific antibody) means that the binding agent has binding specificities for at least two different antigens (e.g., B7H3 and GD2) or at least two different epitopes on the same antigen (e.g., a bispecific antibody directed to B7H3 with a first binding site for a first epitope of a B7H3, and a second binding site for a second epitope of B7H3).

The term “bispecific” when used in reference to a binding agent (e.g., an antibody) means that the binding agent is able to specifically bind to two distinct antigenic determinants, for example, two binding sites each formed by a pair of an antibody heavy chain variable region (VH) and an antibody light chain variable region (VL) binding to different antigens or to different epitopes on the same antigen. When a bispecific binding agent (e.g., an antibody) comprises two antigen binding sites, each may bind to a different antigenic determinant.

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software that can be used to obtain alignments of amino acid or nucleotide sequences are well-known in the art. These include, but are not limited to, BLAST, ALIGN, Megalign, BestFit, GCG Wisconsin Package, and variants thereof. In some embodiments, two nucleic acids or polypeptides are substantially identical, meaning they have at least 70%, at least 75%, at least 80%, at least 85%, at least 90%, and in some embodiments at least 95%, 96%, 97%, 98%, 99% nucleotide or amino acid residue identity, when compared and aligned for maximum correspondence, as measured using a sequence comparison algorithm or by visual inspection. In some embodiments, identity exists over a region of the amino acid sequences that is at least about 10 residues, at least about 20 residues, at least about 40-60 residues, at least about 60-80 residues in length or any integral value there between. In some embodiments, identity exists over a longer region than 60-80 residues, such as at least about 80-100 residues, and in some embodiments the sequences are substantially identical over the full length of the sequences being compared, such as the coding region of a target protein or an antibody. In some embodiments, identity exists over a region of the nucleotide sequences that is at least about 10 bases, at least about 20 bases, at least about 40-60 bases, at least about 60-80 bases in length or any integral value there between. In some embodiments, identity exists over a longer region than 60-80 bases, such as at least about 80-1000 bases or more, and in some embodiments the sequences are substantially identical over the full length of the sequences being compared, such as a nucleotide sequence encoding a protein of interest.

A “conservative amino acid substitution” is one in which one amino acid 'residue is replaced with another amino acid residue having a side chain with similar chemical characteristics. Families of amino acid residues having similar side chains have been generally defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. Generally, conservative substitutions in the sequences of the polypeptides, soluble proteins, and/or antibodies of the disclosure do not abrogate the binding of the polypeptide, soluble protein, or antibody containing the amino acid sequence, to the target binding site. Methods of identifying amino acid conservative substitutions which do not eliminate binding are well-known in the art.

The terms “polypeptide” refers to polymers of amino acids of any length. The polymer can be linear or branched, it can comprise modified amino acids, and it can include (e.g., be interrupted by) non-amino acids. The terms also encompass an amino acid polymer that has been modified naturally or by intervention; for example, disulfide bond formation, glycosylation, lipidation, acetylation, phosphorylation, or any other manipulation or modification, such as linkage to or conjugation with (directly or indirectly) a moiety such as a labeling component. Also included within the definition are, for example, polypeptides containing one or more analogs of an amino acid (including, for example, unnatural amino acids), as well as other modifications known in the art. It is understood that, because the polypeptides of this disclosure can be based upon antibodies or other members of the immunoglobulin superfamily, in some embodiments, the polypeptides can occur as single chains.

As used herein, an “antigen” is a moiety or molecule that contains an epitope to which a binding agent (e.g., an antibody, such as a bispecific antibody) can bind. As such, an antigen can be bound by an antibody. In some embodiments, the antigen, to which a binding agent (e.g., an antibody, such as a bispecific antibody) described herein binds, is B7H3 (e.g., human B7H3), or a fragment thereof.

As used herein, an “epitope” is a term in the art and refers to a localized region of an antigen to which an antibody can bind. An epitope can be a linear epitope or a conformational, non-linear, or discontinuous, epitope. In the case of a polypeptide antigen, for example, an epitope can be contiguous amino acids of the polypeptide (a “linear” epitope) or an epitope can comprise amino acids from two or more non-contiguous regions of the polypeptide (a “conformational,” “non-linear” or “discontinuous” epitope), e.g., human B7H3. It will be appreciated by one of skill in the art that, in general, a linear epitope may or may not be dependent on secondary, tertiary, or quaternary structure. For example, in some embodiments, an antibody binds to a group of amino acids regardless of whether they are folded in a natural three dimensional protein structure. In other embodiments, an antibody requires amino acid residues making up the epitope to exhibit a particular conformation (e.g., bend, twist, turn or fold) in order to recognize and bind the epitope.

An antibody binds “an epitope” or “essentially the same epitope” or “the same epitope” as a reference antibody, when the two antibodies recognize identical, overlapping or adjacent epitopes in a three-dimensional space. The most widely used and rapid methods for determining whether two antibodies bind to identical, overlapping or adjacent epitopes in a three-dimensional space are competition assays, which can be configured in a number of different formats, for example, using either labeled antigen or labeled antibody. In some assays, the antigen is immobilized on a 96-well plate, or expressed on a cell surface, and the ability of unlabeled antibodies to block the binding of labeled antibodies is measured using radioactive, fluorescent or enzyme labels.

“Epitope binning” is the process of grouping antibodies based on the epitopes they recognize. More particularly, epitope binning comprises methods and systems for discriminating the epitope recognition properties of different antibodies, using competition assays combined with computational processes for clustering antibodies based on their epitope recognition properties and identifying antibodies having distinct binding specificities.

As used herein, the terms “specifically binds,” “specifically recognizes,” “immunospecifically binds,” “selectively binds,” “immunospecifically recognizes” and “immunospecific” are analogous terms in the context of antibodies and refer to molecules that bind to an antigen (e.g., epitope) as such binding is understood by one skilled in the art. In some embodiments, “specifically binds” means, for instance that a polypeptide or molecule interacts more frequently, more rapidly, with greater duration, with greater affinity, or with some combination of the above to the epitope, protein, or target molecule than with alternative substances, including related and unrelated proteins. For example, a molecule that specifically binds to an antigen may bind to other peptides or polypeptides, generally with lower affinity as determined by, e.g., immunoassays, Biacore™, KinExA 3000 instrument (Sapidyne Instruments, Boise, ID), or other assays known in the art. In some embodiments, an antibody or antigen binding domain binds to or specifically binds to an antigen when it binds to an antigen with higher affinity than to any cross-reactive antigen as determined using experimental techniques, such as radioimmunoassays (RIA) and enzyme linked immunosorbent assays (ELISAs). Typically, a specific or selective reaction will be at least twice background signal or noise and may be more than 10 times background. See, e.g., Fundamental Immunology 332-36 (Paul ed., 2d ed. 1989) for a discussion regarding binding specificity. In some embodiments, the extent of binding of an antibody or antigen binding domain to a “non-target” protein is less than about 10% of the binding of the antibody or antigen binding domain to its particular target antigen, for example, as determined by fluorescence activated cell sorting (FACS) analysis or RIA. In some embodiments, molecules that specifically bind to an antigen bind to the antigen with a Ka that is at least 2 logs, 2.5 logs, 3 logs, 4 logs or greater than the Ka when the molecules bind to another antigen. In some embodiments, molecules that specifically bind to an antigen do not cross react with other proteins. In another specific embodiment, molecules that specifically bind to an antigen do not cross react with other non-B7H3 proteins. In some embodiments “specifically binds” means, for instance, that a polypeptide or molecule binds a protein or target with a KD of about 0.1 mM or less, but more usually less than about 1 μM. In some embodiments, “specifically binds” means that a polypeptide or molecule binds a target with a KD of at least about 0.1 μM or less, at least about 0.01 μM or less, or at least about 1 nM or less. Because of the sequence identity between homologous proteins in different species, specific binding can include a polypeptide or molecule that recognizes a protein or target in more than one species. Likewise, because of homology within certain regions of polypeptide sequences of different proteins, specific binding can include a polypeptide or molecule that recognizes more than one protein or target. It is understood that, in some embodiments, a polypeptide or molecule that specifically binds a first target may or may not specifically bind a second target. As such, “specific binding” does not necessarily require (although it can include) exclusive binding, e.g., binding to a single target. Thus, a polypeptide or molecule can, in some embodiments, specifically bind more than one target. In some embodiments, multiple targets can be bound by the same antigen-binding site on the polypeptide or molecule. For example, an antibody can, in certain instances, comprise two identical antigen-binding sites, each of which specifically binds the same epitope on two or more proteins. In certain alternative embodiments, an antibody can be bispecific and comprise at least two antigen-binding sites with differing specificities. Generally, but not necessarily, reference to “binding” means “specific binding”.

“Binding affinity” generally refers to the strength of the sum total of noncovalent interactions between a single binding site of a binding molecule (e.g., a binding protein such as an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a binding molecule X for its binding partner Y can generally be represented by the dissociation constant (Ko). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present disclosure. In one embodiment, the “K_{D}” or “K_{D} value” may be measured by assays known in the art, for example by a binding assay. The Ko values reported herein were determined by biolayer interferometry (BLI) using, for example, the OctetQK384 system (ForteBio, Menlo Park, CA). Alternatively, the Ko may also be measured in a radiolabeled antigen binding assay (RIA), for example, performed with the Fab version of an antibody of interest and its antigen (Chen, et al., (1999) J. Mol Biol 293:865-881) or using surface plasmon resonance (SPR) assays by Biacore, using, for example, a BIAcore™-2000 or a BIAcore™-3000 BIAcore, Inc., Piscataway, NJ). An “on-rate” or “rate of association” or “association rate” or “kₒₙ,” as well as an “off-rate” or “rate of dissociation” or “dissociation rate” or “k_{off},” may can also be determined with the same SPR or BLI techniques described above using, for example, the OctetQK384 sytem (ForteBio, Menlo Park, CA) or a BIAcore™-2000 or a BIAcore™-3000 (BIAcore, Inc., Piscataway, NJ), respectively.

“Binding avidity” generally refers to the overall binding strength of the total of all noncovalent interactions between all the binding sites of a binding molecule (e.g., a binding protein such as a bispecific antibody) and its binding partner(s) (e.g., antigen(s)). Binding affinity is one factor that influences the avidity of the interaction between a molecule and its binding partner(s). Other factors that can influence binding avidity include valency of the binding partner and the binding molecule, density of the binding molecule on the cellular surface, as well as the structural arrangement of their interaction. For example, if both the binding molecule and the binding partner(s) are multivalent and there is a favorable structural arrangement between them, the interaction between the binding molecule and the binding partner(s) can be very strong due to high avidity, despite one or more of the individual binding affinities of the binding molecule having low individual binding affinity to a single binding partner. A variety of methods of measuring binding avidity are known in the art, any of which can be used for purposes of the present disclosure. Examples of methods for measuring binding avidity of binding molecules (e.g., a binding protein such as a bispecific antibody) include solid-phase radioimmunoassay, surface plasmon resonance, cell binding, and modified ELISA as described in Correa et al., Biomed. J., 44(4):433-438 (2021), Brady et al., J. Immunol. Methods., 447:31-36 (2017), Hedman et al., Rev. Med. Microbiol., 4:123-129 (1993), and Lynch et al., J Immunol Methods., 404:1-12 (2014).

As used herein, the term “constant region” or “constant domain” is a well-known antibody term of art and refers to an antibody portion, e.g., for example, a carboxyl terminal portion of a light and/or heavy chain which is not directly involved in binding of an antibody to an antigen but which can exhibit various effector functions, such as interaction with the Fc receptor. The term includes the portion of an immunoglobulin molecule having a generally more conserved amino acid sequence relative to an immunoglobulin variable region.

Antibody “effector functions” refer to those biological activities attributable to the Fc region (e.g., a native sequence Fc region or amino acid sequence variant Fc region) of an antibody, and vary with the antibody isotype. Examples of antibody effector functions include: C1q binding and complement dependent cytotoxicity; Fc receptor binding; antibody-dependent cell-mediated cytotoxicity (ADCC); phagocytosis; down regulation of cell surface receptors (e.g., B cell receptor); and B cell activation.

The term “Fc region” herein is used to define a C-terminal region of an immunoglobulin heavy chain, including, for example, native sequence Fc regions, recombinant Fc regions, and variant Fc regions. Although the boundaries of the Fc region of an immunoglobulin heavy chain might vary, the human IgG heavy chain Fc region is often defined to stretch from an amino acid residue at position Cys226 (according to the EU numbering system), or from Pro230 (according to the EU numbering system), to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. A first exemplary Fc region sequence is provided below (CH2 domain=bold text; CH3 domain=underline text):

(SEQ ID NO: 81)
CPPCPAPELLGGPSVFLFPPKPKDTLMISRTPEVTCVVVDVSHEDPEV
KFNWYVDGVEVHNAKTKPREEQYNSTYRVVSVLTVLHQDWLNGKEYKC
KVSNKALPAPIEKTISKAKGQPREPQVYTLPPSRDELTKNQVSLTCLV
KGFYPSDIAVEWESNGQPENNYKTTPPVLDSDGSFFLYSKLTVDKSRW
QQGNVFSCSVMHEALHNHYTQKSLSLSPGK.

A second exemplary Fc region sequence is provided below (CH2 domain=bold text; CH3 domain=underline text):

(SEQ ID NO: 82)
CPPCPAPELLGGPSVFLFPPKPKDTLMISRTPEVTCVVVDVSHEDPEV
KFNWYVDGVEVHNAKTKPREEQYNSTYRVVSVLTVLHQDWLNGKEYKC
KVSNKALPAPIEKTISKAKGQPREPQVYTLPPSRDELTKNQVSLWCLV
KGFYPSDIAVEWESNGQPENNYKTTPPVLDSDGSFFLYSKLTVDKSRW
QQGNVFSCSVMHEALHNHYTQKSLSLSPGK.

A third exemplary Fc region sequence is provided below (CH2 domain=bold text; CH3 domain=underline text):

(SEQ ID NO: 83)
CPPCPAPELLGGPSVFLFPPKPKDTLMISRTPEVTCVVVDVSHEDPEV
KFNWYVDGVEVHNAKTKPREEQYNSTYRVVSVLTVLHQDWLNGKEYKC
KVSNKALPAPIEKTISKAKGQPREPQVYTLPPSREEMTKNQVSLSCAV
KGFYPSDIAVEWESNGQPENNYKTTPPVLDSDGSFFLVSKLTVDKSRW
QQGNVFSCSVMHEALHNHYTQKSLSLSPGK.

A “functional Fc region” possesses an “effector function” of a native sequence Fc region. Exemplary “effector functions” include C1q binding; complement dependent cytotoxicity (CDC); Fc receptor binding; antibody-dependent cell-mediated cytotoxicity (ADCC); phagocytosis; down regulation of cell surface receptors (e.g., B cell receptor; BCR), etc. Such effector functions generally require the Fc region to be combined with a binding region or binding domain (e.g., an antibody variable region) and can be assessed using various assays as disclosed.

A “native sequence Fc region” comprises an amino acid sequence identical to the amino acid sequence of an Fc region found in nature, and not manipulated, modified, and/or changed (e.g., isolated, purified, selected, including or combining with other sequences such as variable region sequences) by a human. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification, (e.g., substituting, addition, or deletion) preferably one or more amino acid substitution(s). In some embodiments, the variant Fc region has at least one amino acid substitution compared to a native sequence Fc region or to the Fc region of a parent polypeptide, for example, from about one to about ten amino acid substitutions, and preferably from about one to about five amino acid substitutions in a native sequence Fc region or in the Fc region of the parent polypeptide. The variant Fc region herein can possess at least about 80% homology with a native sequence Fc region and/or with an Fc region of a parent polypeptide, or at least about 90% homology therewith, for example, at least about 95% homology therewith. The variant Fc region herein described herein may have modified effector function. An first exemplary variant Fc region sequence is provided below (CH2 domain=bold text with amino acid changes underlined; CH3 domain=underline text):

(SEQ ID NO: 84)
CPPCPAPEAAGGPSVFLFPPKPKDTLMISRTPEVTCVVVDVSHEDPEV
KFNWYVDGVEVHNAKTKPREEQYNSTYRVVSVLTVLHQDWLNGKEYKC
KVSNKALKAPIEKTISKAKGQPREPQVYTLPPSRDELTKNQVSLTCLV
KGFYPSDIAVEWESNGQPENNYKTTPPVLDSDGSFFLYSKLTVDKSRW
QQGNVFSCSVMHEALHNHYTQKSLSLSPGK.

An second exemplary variant Fc region sequence is provided below (CH2 domain=bold text with amino acid changes underlined; CH3 domain=underline text):

(SEQ ID NO: 85)
CPPCPAPEAAGGPSVFLFPPKPKDTLMISRTPEVTCVVVDVSHEDPEV
KFNWYVDGVEVHNAKTKPREEQYNSTYRVVSVLTVLHQDWLNGKEYKC
KVSNKALKAPIEKTISKAKGQPREPQVYTLPPSRDELTKNQVSLWCLV
KGFYPSDIAVEWESNGQPENNYKTTPPVLDSDGSFFLYSKLTVDKSRW
QQGNVFSCSVMHEALHNHYTQKSLSLSPGK.

An third exemplary variant Fc region sequence is provided below (CH2 domain=bold text with amino acid changes underlined; CH3 domain=underline text):

(SEQ ID NO: 86)
CPPCPAPEAAGGPSVFLFPPKPKDTLMISRTPEVTCVVVDVSHEDPEV
KFNWYVDGVEVHNAKTKPREEQYNSTYRVVSVLTVLHQDWLNGKEYKC
KVSNKALKAPIEKTISKAKGQPREPQVYTLPPSREEMTKNQVSLSCAV
KGFYPSDIAVEWESNGQPENNYKTTPPVLDSDGSFFLVSKLTVDKSRW
QQGNVFSCSVMHEALHNHYTQKSLSLSPGK.

An exemplary variant CH2 domain (e.g., silent CH2) sequence useful for multispecific (e.g., bispecific) binding agents described herein is provided below (amino acid changes underlined):

(SEQ ID NO: 87)
APEAAGGPSVFLFPPKPKDTLMISRTPEVTCVVVDVSHEDPEVKFNWY
VDGVEVHNAKTKPREEQYNSTYRVVSVLTVLHQDWLNGKEYKCKVSNK
ALKAPIEKTISKAK.

As used herein, the term “heavy chain” when used in reference to an antibody refers to a polypeptide chain of about 50-70 kDa, wherein the amino-terminal portion includes a variable region of about 120 to 130 or more amino acids, and a carboxy-terminal portion includes one or more constant regions. The “heavy chain” can refer to any distinct types, e.g., for example, alpha (a), delta (6), epsilon (s), gamma (γ) and mu (p), based on the amino acid sequence of the constant domain, which give rise to IgA, IgD, IgE, IgG and IgM classes of antibodies, respectively, including subclasses of IgG, e.g., IgG1, IgG2, IgG3 and IgG4.

As used herein, the term “light chain” when used in reference to an antibody can refer to a polypeptide chain of about 25 kDa, wherein the amino-terminal portion includes a variable region of about 100 to about 110 or more amino acids, and a carboxy-terminal portion includes a constant region. The approximate length of a light chain is 211 to 217 amino acids. There are two distinct types, e.g., kappa (κ) of lambda (λ) based on the amino acid sequence of the constant domains. Light chain amino acid sequences are well known in the art.

The terms “antigen binding domain,” “binding domain,” and similar terms refer to that portion of a binding agent (e.g., an antibody, including a bispecific antibody) that comprises the amino acid residues that interact with an antigen and confer on the binding agent, domain, or region its specificity and affinity for the antigen (e.g., the CDRs). An antigen binding domain and the binding agent comprising an antigen binding domain can be referred to as recognizing the antigen or epitope to which the antigen binding domain specifically binds. Likewise, the antigen or epitope can be referred to as being the determinant of the recognition specificity or binding specificity of the antigen binding domain.

Antibodies described herein include, but are not limited to, synthetic antibodies, monoclonal antibodies, recombinantly produced antibodies, multispecific antibodies (e.g., including bispecific antibodies), human antibodies, humanized antibodies, chimeric antibodies, intrabodies, single-chain Fvs (scFv) (e.g., including monospecific, bispecific, etc.), camelized antibodies, Fab fragments, F(ab′) fragments, disulfide-linked Fvs (sdFv), anti-idiotypic (anti-Id) antibodies, and epitope-binding fragments of any of the above.

In some embodiments, antibodies described herein include immunoglobulin molecules and immunologically active portions of immunoglobulin molecules, including molecules that contain one or more antigen binding domains that bind to a B7H3 antigen and GD2.

Antibodies can be of any type (e.g., IgG, IgE, IgM, IgD, IgA or IgY), any class, (e.g., IgG1, IgG2, IgG3, IgG4, IgA1 or IgA2), or any subclass (e.g., IgG2a or IgG2b) of immunoglobulin molecule. In some embodiments, antibodies described herein are IgG antibodies (e.g., human IgG), or a class (e.g., human IgG1, IgG2, IgG3 or IgG4) or subclass thereof.

In some embodiments, an antibody is a 4-chain antibody unit comprising two heavy (H) chain/light (L) chain pairs, wherein the amino acid sequences of the H chains are identical and the amino acid sequences of the L chains are identical. In some embodiments, the H and L chains comprise constant regions, for example, human constant regions. In some embodiments, the L chain constant region of such antibodies is a kappa or lambda light chain constant region, for example, a human kappa or lambda light chain constant region. In some embodiments, the H chain constant region of such antibodies comprise a gamma heavy chain constant region, for example, a human gamma heavy chain constant region. In some embodiments, such antibodies comprise IgG constant regions, for example, human IgG constant regions (e.g., IgG1, IgG2, IgG3, and/or IgG4 constant regions).

A multispecific binding agent (e.g., an antibody, such as a bispecific antibody) may preferentially bind to B7H3, such as human B7H3, meaning that the multispecific binding agent binds B7H3 with greater affinity than it binds to an unrelated control protein and/or binds human B7H3 with greater affinity than it binds to an unrelated control protein. For example, the multispecific binding agent may specifically recognize and bind B7H3 or a portion thereof. Similarly, multispecific binding agent (e.g., an antibody, such as a bispecific antibody) may preferentially bind to GD2, such as human GD2, meaning that the multispecific binding agent binds GD2 with greater affinity than it binds to an unrelated control protein and/or binds human B7H3 with greater affinity than it binds to an unrelated control protein. For example, the multispecific binding agent may specifically recognize and bind GD2 or a portion thereof. Likewise, multispecific binding agent (e.g., an antibody, such as a bispecific antibody) may preferentially bind to targets having both B7H3 and GD2 (e.g., cells co-expressing B7H3, such as human B7H3, and GD2, such as human GD2), meaning that the multispecific binding agent binds to targeting having B7H3 and GD2 with greater affinity than it binds to targets having an unrelated control protein and/or only B7H3 or GD2 (e.g., cells expressing B7H3, but not GD2, or cells expressing GD2, but not B7H3). For example, the multispecific binding agent may specifically recognize and bind cells co-expressing both B7H3 and GD2 as compared to cells expressing GD2, but not B7H3. “Specific binding” means that the multispecific binding agent (e.g., an antibody, such as a bispecific antibody) thereof binds to B7H3 and/or GD2 with an affinity that is at least 5, 10, 15, 20, 25, 50, 100, 250, 500, 1000, or 10,000 times greater than the affinity for an unrelated control protein (e.g., hen egg white lysozyme) or to targets having only B7H3 or GD2. In some embodiments, the multispecific binding agent (e.g., an antibody, such as a bispecific antibody) thereof may bind B7H3 and/or GD2 substantially exclusively (e.g., is able to distinguish B7H3 and/or GD2 from other known polypeptides, for example, by virtue of measurable differences in binding affinity). In some embodiments, a multispecific binding agent (e.g., an antibody, such as a bispecific antibody) may react with B7H3 sequences other than human B7H3 sequences (e.g., cynomolgus B7H3 sequences). In some embodiments, a multispecific binding agent (e.g., an antibody, such as a bispecific antibody) may react with GD2 sequences other than human GD2 sequences (e.g., cynomolgus B7H3 sequences).

The term “variable region” or “variable domain” refers to a portion of the light or heavy chains of an antibody that is generally located at the amino-terminal of the light or heavy chain and has a length of about 120 to 130 amino acids in the heavy chain and about 100 to 110 amino acids in the light chain, and are used in the binding and specificity of each particular antibody for its particular antigen. The variable region of the heavy chain may be referred to as “VH.” The variable region of the light chain may be referred to as “VL.” The term “variable” refers to the fact that certain segments of the variable regions differ extensively in sequence among antibodies. The V region mediates antigen binding and defines specificity of a particular antibody for its particular antigen. However, the variability is not evenly distributed across the 110-amino acid span of the variable regions. Instead, the V regions consist of less variable (e.g., relatively invariant) stretches called framework regions (FRs) of about 15-30 amino acids separated by shorter regions of greater variability (e.g., extreme variability) called “hypervariable regions” or alternatively called “complementarity determining regions.” The variable regions of heavy and light chains each comprise four FRs (FR1, FR2, FR3 and FR4), largely adopting a p sheet configuration, connected by three hypervariable regions, which form loops connecting, and in some cases forming part of, the p sheet structure. The hypervariable regions in each chain are held together in close proximity by the FRs and, with the hypervariable regions from the other chain, contribute to the formation of the antigen-binding site of antibodies (see, e.g., Kabat et al., Sequences of Proteins of Immunological Interest, 5th Ed. Public Health Service, National Institutes of Health, Bethesda, MD, 1991)). The constant regions are not involved directly in binding an antibody to an antigen, but exhibit various effector functions, such as participation of the antibody in ADCC and CDC. The variable regions differ extensively in sequence between different antibodies. The variability in sequence is concentrated in the CDRs while the less variable portions in the variable region are referred to as framework regions (FR). The CDRs of the light and heavy chains are primarily responsible for the interaction of the antibody with antigen. In specific embodiments, the variable region is a human variable region.

The term “hypervariable region,” “HVR,” “HV,” “complementarity determining region,” or “CDR” when used herein refers to the regions of an antibody variable region that are hypervariable in sequence and/or form structurally defined loops. Generally, antibodies comprise six hypervariable regions; three in the VH (H1, H2, H3), and three in the VL (L1, L2, L3). A number of hypervariable region delineations are in use and are encompassed herein. The Kabat CDRs are based on sequence variability and are the most commonly used (see, e.g., Kabat et al., Sequences of Proteins of Immunological Interest, 5th Ed. Public Health Service, National Institutes of Health, Bethesda, MD. (1991)). Chothia refers instead to the location of the structural loops (see, e.g., Chothia and Lesk, J. Mol. Biol. 196:901-917 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software (see, e.g., Martin, in Antibody Engineering, Vol. 2, Chapter 3, Springer Verlag). The “contact” hypervariable regions are based on an analysis of the available complex crystal structures. The residues from each of these hypervariable regions or CDRs are noted below.

A universal numbering system has been developed and widely adopted, ImMunoGeneTics (IMGT) Information System® (Lefranc et al., Dev. Comp. Immunol. 27(1):55-77 (2003)). IMGT is an integrated information system specializing in immunoglobulins (IG), T cell receptors (TR) and major histocompatibility complex (MHC) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues and are readily identified. This information can be used in grafting and replacement of CDR residues from immunoglobulins of one species into an acceptor framework from, typically, a human antibody. An additional numbering system (AHon) has been developed by Honegger and Pluckthun, J. Mol. Biol. 309: 657-670 (2001). Correspondence between the numbering system, including, for example, the Kabat numbering and the IMGT unique numbering system, is well known to one skilled in the art (see, e.g., Kabat, supra; Chothia and Lesk, supra; Martin, supra; Lefranc et al., supra) and is also illustrated below. An Exemplary system, shown herein, combines Kabat and Chothia.

| | Exemplary | IMGT | Kabat | AbM | Chothia | Contact |
|---|---|---|---|---|---|---|
| V_{H} CDR1 | 26-35 | 27-38 | 31-35 | 26-35 | 26-32 | 30-35 |
| V_{H} CDR2 | 50-65 | 56-65 | 50-65 | 50-58 | 52a/ | 47-58 |
| | | | | | 53-55 | |
| V_{H} CDR3 | 95-102 | 105-117 | 95-102 | 95-102 | 96-101 | 93-101 |
| V_{L} CDR1 | 24-34 | 27-38 | 24-34 | 24-34 | 26-32 | 30-36 |
| V_{L} CDR2 | 50-56 | 56-65 | 50-56 | 50-56 | 50-52 | 46-55 |
| V_{L} CDR3 | 89-97 | 105-117 | 89-97 | 89-97 | 91-96 | 89-96 |

Hypervariable regions may comprise “extended hypervariable regions” as follows: 24-36 or 24-34 (L1), 46-56 or 50-56 (L2) and 89-97 or 89-96 (L3) in the VL and 26-35 or 26-35A (H1), 50-65 or 49-65 (H2) and 93-102, 94-102, or 95-102 (H3) in the VH. As used herein, the terms “hypervariable region,” “HVR,” “HV,” “complementarity determining region,” or “CDR” are used interchangeably.

The term “vector” refers to a substance that is used to carry or include a nucleic acid sequences, including for example, in order to introduce a nucleic acid sequence into a host cell. Vectors applicable for use include, for example, expression vectors, plasmids, phage vectors, viral vectors, episomes and artificial chromosomes, which can include selection sequences or markers operable for stable integration into a host cell's chromosome. Additionally, the vectors can include one or more selectable marker genes and appropriate expression control sequences. Selectable marker genes that can be included, for example, provide resistance to antibiotics or toxins, complement auxotrophic deficiencies, or supply critical nutrients not in the culture media. Expression control sequences can include constitutive and inducible promoters, transcription enhancers, transcription terminators, and the like which are well known in the art. When two or more nucleic acid molecules are to be co-expressed (e.g., both an antibody heavy and light chain or an antibody VH and VL) both nucleic acid molecules can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The introduction of nucleic acid molecules into a host cell can be confirmed using methods well known in the art. Such methods include, for example, nucleic acid analysis such as Northern blots or polymerase chain reaction (PCR) amplification of mRNA, or immunoblotting for expression of gene products, or other suitable analytical methods to test the expression of an introduced nucleic acid sequence or its corresponding gene product. It is understood by those skilled in the art that the nucleic acid molecules are expressed in a sufficient amount to produce a desired product (e.g., a multispecific binding agent as described herein), and it is further understood that expression levels can be optimized to obtain sufficient expression using methods well known in the art.

An “effective amount” is generally an amount sufficient to reduce the severity and/or frequency of symptoms, eliminate the symptoms and/or underlying cause, prevent the occurrence of symptoms and/or their underlying cause, and/or improve or remediate the damage that results from or is associated with a disease, disorder, or condition. In some embodiments, the effective amount is a therapeutically effective amount or a prophylactically effective amount.

The term “therapeutically effective amount” as used herein refers to the amount of an agent (e.g., a bispecific antibody described herein or any other agent described herein) that is sufficient to reduce and/or ameliorate the severity and/or duration of a given disease, disorder or condition, and/or a symptom related thereto. A therapeutically effective amount of an agent, including a therapeutic agent, can be an amount necessary for (i) reduction or amelioration of the advancement or progression of a given disease, disorder, or condition, (ii) reduction or amelioration of the recurrence, development or onset of a given disease, disorder or conditions, and/or (iii) to improve or enhance the prophylactic or therapeutic effect of another therapy (e.g., a therapy other than the administration of an antibody described herein). A “therapeutically effective amount” of a substance/molecule/agent of the present disclosure (e.g., a bispecific antibody) may vary according to factors such as the disease state, age, sex, and weight of the individual, and the ability of the substance/molecule/agent, to elicit a desired response in the individual. A therapeutically effective amount encompasses an amount in which any toxic or detrimental effects of the substance/molecule/agent are outweighed by the therapeutically beneficial effects. In certain embodiments, the term “therapeutically effective amount” refers to an amount of an antibody or other agent (e.g., or drug) effective to “treat” a disease, disorder, or condition, in a subject or mammal.

A “prophylactically effective amount” is an amount of a pharmaceutical composition that, when administered to a subject, will have the intended prophylactic effect, e.g., preventing or delaying the onset (or reoccurrence) of a disease, disorder or condition, or reducing the likelihood of the onset (or reoccurrence) of a disease, disorder, or condition or associated symptom(s). The full therapeutic or prophylactic effect does not necessarily occur by administration of one dose, and may occur only after administration of a series of doses. Thus, a therapeutically or prophylactically effective amount may be administered in one or more administrations.

The term “pharmaceutically acceptable” as used herein means being approved by a regulatory agency of the Federal or a state government, or listed in the U.S. Pharmacopeia, European Pharmacopeia or other generally recognized Pharmacopeia for use in animals, and more particularly in humans.

“Carriers” as used herein include carriers, excipients, or stabilizers that are nontoxic to the cell or mammal being exposed thereto at the dosages and concentrations employed. Often the carrier is an aqueous pH buffered solution. Examples of carriers include buffers such as phosphate, citrate, and other organic acids; antioxidants including ascorbic acid; low molecular weight ((e.g., less than about 10 amino acid residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ polyethylene glycol (PEG), and PLURONICS™. The term “carrier” can also refer to a diluent, adjuvant (e.g., Freund's adjuvant (complete or incomplete)), excipient, or vehicle with which the therapeutic is administered. Such carriers can be sterile liquids, such as water and oils, including those of petroleum, animal, vegetable or synthetic origin, such as peanut oil, soybean oil, mineral oil, sesame oil and the like. Water is an exemplary carrier when a composition (e.g., a pharmaceutical composition) is administered intravenously. Saline solutions and aqueous dextrose and glycerol solutions can also be employed as liquid carriers, particularly for injectable solutions. Suitable excipients (e.g., pharmaceutical excipients) include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. Compositions can take the form of solutions, suspensions, emulsion, tablets, pills, capsules, powders, sustained-release formulations and the like. Oral compositions, including formulations, can include standard carriers such as pharmaceutical grades of mannitol, lactose, starch, magnesium stearate, sodium saccharine, cellulose, magnesium carbonate, etc. Examples of suitable carriers are described in Remington's Pharmaceutical Sciences (1990) Mack Publishing Co., Easton, PA. Compositions, including pharmaceutical compounds, may contain a prophylactically or therapeutically effective amount of a multispecific binding agent (e.g., an antibody, such as a bispecific antibody), for example, in isolated or purified form, together with a suitable amount of carrier so as to provide the form for proper administration to the subject (e.g., patient). The formulation should suit the mode of administration.

In some embodiments, the present disclosure provides multispecific binding agents that can be used herein as therapeutic agents. Such agents include multispecific antibodies (e.g., antibodies, such as bispecific antibodies) comprising a first binding domain that binds to B7H3, including human B7H3, and a second binding domain that binds GD2, including human GD2. Exemplary antibodies include humanized, human, bispecific, and heteroconjugate antibodies, as well as variants thereof having increased or decreased affinity or other properties.

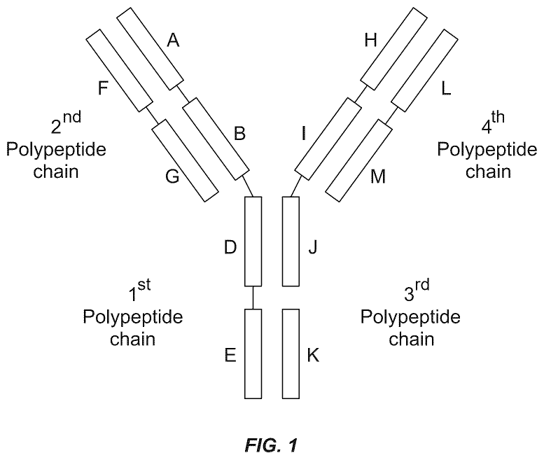

In some embodiments, the present disclosure also provides multispecific binding agents (e.g., antibodies, such as bispecific antibodies) as an antibody construct as described in FIG. 1. For example, in some embodiments, multispecific binding agents (e.g., antibodies, such as bispecific antibodies) described herein include four polypeptide chains, wherein the first polypeptide chain includes Domains A-B-D-E, the second polypeptide chain includes Domains F-G, the third polypeptide chain includes Domains H-I-J-K, and the fourth polypeptide chain includes Domains L-M. In some embodiments, Domain A is a VL region described herein. In some embodiments, Domain B is a variant CH3 described herein (e.g., SEQ ID NO: 73). In some embodiments, Domain D is a CH2 described herein (e.g., SEQ ID NO: 74). In some embodiments, Domain E is a variant CH3 described herein (e.g., SEQ ID NO: 75). In some embodiments, Domain F is a VH region described herein. In some embodiments, Domain G is a variant CH3 described herein (e.g., SEQ ID NO: 70). In some embodiments, Domain H is a VL described herein. In some embodiments, Domain I is a CL (e.g., Kappa) described herein (e.g., SEQ ID NO: 76). In some embodiments, Domain J is a CH2 described herein (e.g., SEQ ID NO: 77). In some embodiments, Domain K is a variant CH3 described herein (e.g., SEQ ID NO: 78). In some embodiments, Domain L is a VH region. In some embodiments, Domain M is a CH1 described herein (e.g., SEQ ID NO: 72).

In some embodiments, multispecific binding agents (e.g., antibodies, such as bispecific antibodies) described herein include a first polypeptide chain having domains connected (e.g., via a peptide bond) in a specific order. For example, as described herein as “Domains A-B-D-E,” the C-terminus of Domain A is connected to the N-terminus of Domain B, the C-terminus of Domain B is connected to the N-terminus of Domain D, and the C-terminus of Domain D is connected to the N-terminus of Domain E. Accordingly, in some embodiments, a multispecific binding agent (e.g., an antibody, such as a bispecific antibody) includes a first polypeptide chain having a VL region described herein (Domain A) connected to constant regions (Domains B-D-E), wherein the constant regions include the amino acid sequence of SEQ ID NO: 69. In some embodiments, multispecific binding agent (e.g., an antibody, such as a bispecific antibody) includes a first polypeptide chain having the amino acid sequence of SEQ ID NO: 53. In some embodiments, multispecific binding agent (e.g., an antibody, such as a bispecific antibody) includes a first polypeptide chain having the amino acid sequence of SEQ ID NO: 57.

In some embodiments, multispecific binding agents (e.g., antibodies, such as bispecific antibodies) described herein include a second polypeptide chain having domains connected (e.g., via a peptide bond) in a specific order. For example, as described herein as “Domains F-G,” the C-terminus of Domain F is connected to the N-terminus of Domain G. Accordingly, in some embodiments, a multispecific binding agent (e.g., an antibody, such as a bispecific antibody) includes a second polypeptide chain having a VH region described herein (Domain F) connected to a constant region (Domain G), wherein the constant region includes the amino acid sequence of SEQ ID NO: 70. In some embodiments, multispecific binding agent (e.g., an antibody, such as a bispecific antibody) includes a second polypeptide chain having the amino acid sequence of SEQ ID NO: 54. In some embodiments, multispecific binding agent (e.g., an antibody, such as a bispecific antibody) includes a second polypeptide chain having the amino acid sequence of SEQ ID NO: 58.

In some embodiments, multispecific binding agents (e.g., antibodies, such as bispecific antibodies) described herein include a third polypeptide chain having domains connected (e.g., via a peptide bond) in a specific order. For example, as described herein as “Domains H-I-J-K,” the C-terminus of Domain H is connected to the N-terminus of Domain I, the C-terminus of Domain I is connected to the N-terminus of Domain J, and the C-terminus of Domain J is connected to the N-terminus of Domain K. Accordingly, in some embodiments, a multispecific binding agent (e.g., an antibody, such as a bispecific antibody) includes a third polypeptide chain having a VL region described herein (Domain H) connected to constant regions (Domains I-J-K), wherein the constant region includes the amino acid sequence of SEQ ID NO: 71. In some embodiments, multispecific binding agent (e.g., an antibody, such as a bispecific antibody) includes a third polypeptide chain having the amino acid sequence of SEQ ID NO: 55. In some embodiments, multispecific binding agent (e.g., an antibody, such as a bispecific antibody) includes a third polypeptide chain having the amino acid sequence of SEQ ID NO: 59.

In some embodiments, multispecific binding agents (e.g., antibodies, such as bispecific antibodies) described herein include a fourth polypeptide chain having domains connected (e.g., via a peptide bond) in a specific order. For example, as described herein as “Domains L-M,” the C-terminus of Domain L is connected to the N-terminus of Domain M. Accordingly, in some embodiments, a multispecific binding agent (e.g., an antibody, such as a bispecific antibody) includes a fourth polypeptide chain having a VH region described herein (Domain L) connected to a constant region (Domain M), wherein the constant regions include the amino acid sequence of SEQ ID NO: 72. In some embodiments, multispecific binding agent (e.g., an antibody, such as a bispecific antibody) includes a fourth polypeptide chain having the amino acid sequence of SEQ ID NO: 56. In some embodiments, multispecific binding agent (e.g., an antibody, such as a bispecific antibody) includes a fourth polypeptide chain having the amino acid sequence of SEQ ID NO: 60.

In some embodiments, described herein are multispecific binding agents (e.g., antibodies, such as bispecific antibodies) comprising a first binding domain that binds to B7H3, including a B7H3 polypeptide, a B7H3 polypeptide fragment, a B7H3 peptide or a B7H3 epitope. In some embodiments, the multispecific binding agents are human or humanized antibodies (e.g., comprising human constant regions) comprising a first binding domain that binds B7H3, including a B7H3 polypeptide, a B7H3 polypeptide fragment, a B7H3 peptide or a B7H3 epitope. In some embodiments, a multispecific binding agent (e.g., an antibody, such as a bispecific antibody) can bind to B7H3 expressed on the surface of a mammalian (e.g., human) cell, including a B7H3 expressing tumor cell. In some embodiments, a multispecific binding agent (e.g., an antibody, such as a bispecific antibody) binds a B7H3 extracellular epitope exposed on a cell such as a tumor cell (e.g., a B7H3 epitope). In some embodiments, described herein is a multispecific binding agent (e.g., an antibody, such as a bispecific antibody) that binds to B7H3, such as human B7H3 or portions thereof. In some embodiments, B7H3 is a human B7H3. In some embodiments, a multispecific binding agent is a multispecific binding agent that binds to B7H3 (e.g., an antibody that binds to human B7H3). An exemplary amino acid sequence of human B7H3 is described herein.

In some embodiments, described herein are multispecific binding agents (e.g., antibodies, such as bispecific antibodies) comprising a second binding domain that bind to GD2, including a GD2 polypeptide, a GD2 polypeptide fragment, a GD2 peptide or a GD2 epitope. In some embodiments, the multispecific binding agents are humanized antibodies (e.g., comprising human constant regions) comprising a second binding domain that binds GD2, including a GD2 polypeptide, a GD2 polypeptide fragment, a GD2 peptide or a GD2 epitope. In some embodiments, a multispecific binding agent (e.g., an antibody, such as a bispecific antibody) can bind to GD2 expressed on the surface of a mammalian (e.g., human) cell, including a GD2 expressing antigen presenting cells and tumor cells. In some embodiments, a multispecific binding agent (e.g., an antibody, such as a bispecific antibody) binds a GD2 extracellular epitope exposed on a cell such as a tumor cell (e.g., a B7H3 epitope). In some embodiments, described herein is a multispecific binding agent (e.g., an antibody, such as a bispecific antibody) comprising a second binding domain that binds to GD2, such as human GD2 or portions thereof. In some embodiments, GD2 is a human GD2. In some embodiments, a multispecific binding agent is a multispecific binding agent that binds to human GD2 (e.g., an antibody that binds to human GD2). An exemplary amino acid sequence of human GD2 is described herein.

In some embodiments, the multispecific binding agents (e.g., antibodies, such as bispecific antibodies) described herein comprise a VH region, VL region, VH CDR1, VH CDR2, VH CDR3, VL CDR1, VL CDR2, and/or VL CDR3 of any one of the antibodies described herein, such as an amino acid sequence of a VH region, VL region, VH CDR1, VH CDR2, VH CDR3, VL CDR1, VL CDR2, and/or VL CDR3 depicted in Tables 1-2. Accordingly, in some embodiments, the multispecific binding agent (e.g., an antibody, such as a bispecific antibody) described herein comprises a first binding domain that comprises one, two, and/or three heavy chain CDRs and/or one, two, and/or three light chain CDRs from the antibody designated mAb-B7H3, as shown in Table 1. In some embodiments, the multispecific binding agent (e.g., an antibody, such as a bispecific antibody) described herein comprises a second binding domain that comprises one, two, and/or three heavy chain CDRs and/or one, two, and/or three light chain CDRs from the antibody designated mAb-GD2, as shown in Table 2. In some embodiments, the multispecific binding agent (e.g., an antibody, such as a bispecific antibody) described herein comprises a first binding domain that comprises one, two, and/or three heavy chain CDRs and/or one, two, and/or three light chain CDRs from the antibody designated mAb-B7H3, as shown in Table 1, and a second binding domain that comprises one, two, and/or three heavy chain CDRs and/or one, two, and/or three light chain CDRs from the antibody designated mAb-GD2, as shown in Table 2. In some embodiments, the multispecific binding agent (e.g., an antibody, such as a bispecific antibody) described herein comprises a first binding domain that comprises one, two, and/or three heavy chain CDRs and one, two, and/or three light chain CDRs from the antibody designated mAb-B7H3, as shown in Table 1, and a second binding domain that comprises one, two, and/or three heavy chain CDRs and one, two, and/or three light chain CDRs from the antibody designated mAb-GD2, as shown in Table 2.

In some embodiments, a multispecific binding agent (e.g., an antibody, such as a bispecific antibody) comprises a first binding domain that binds to B7H3 and comprises a VH region, which comprises VH CDR1, VH CDR2, and/or VH CDR3, and a VL region, which comprises VL CDR1, VL CDR2, and/or VL CDR3, of any one of the binding agents described in Table 1, and a second binding domain that binds to GD2 and comprises a VH region, which comprises VH CDR1, VH CDR2, and/or VH CDR3, and a VL region, which comprises VL CDR1, VL CDR2, and/or VL CDR3, of any one of the binding agents described in Table 2. Accordingly, in some embodiments, a multispecific binding agent (e.g., an antibody, such as a bispecific antibody) described herein comprises a first binding domain that comprises one, two, and/or three heavy chain CDRs and/or one, two, and/or three light chain CDRs from Table 1. In some embodiments, the multispecific binding agent (e.g., an antibody, such as a bispecific antibody) described herein comprises a second binding domain that comprises one, two, and/or three heavy chain CDRs and/or one, two, and/or three light chain CDRs from Table 2. In some embodiments, a multispecific binding agent (e.g., an antibody, such as a bispecific antibody) described herein is bispecific and comprises a first binding domain that comprises one, two, and/or three heavy chain CDRs and one, two, and/or three light chain CDRs from Table 1, and a second binding domain that comprises one, two, and/or three heavy chain CDRs and one, two, and/or three light chain CDRs from Table 2.

The antibody designated mAb-B7H3 comprises a VH sequence that is SEQ ID NO: 25 and a VL sequence that is SEQ ID NO: 26.

The antibody designated mAb-GD2 comprises a VH sequence that is SEQ ID NO: 51 and a VL sequence that is SEQ ID NO: 52.

In some embodiments, a multispecific binding agent (e.g., an antibody, such as a bispecific antibody) that binds to B7H3 comprises (i) a VH region wherein the VH region comprises a VH sequence that is SEQ ID NO: 25 (mAb-B7H3 VH) and (ii) a VL region wherein the VL region comprises a VL sequence that is SEQ ID NO: 26 (mAb-B7H3 VL). In some embodiments, such VH and VL regions were used to construct bispecific binding agents (e.g., antibodies) each with a first binding domain that bind to B7H3, including wherein the first binding domain comprises the mAb-B7H3 VH and mAb-B7H3 VL regions.

In some embodiments, a multispecific binding agent (e.g., an antibody, such as a bispecific antibody) that binds to GD2 comprises (i) a VH region wherein the VH region comprises a VH sequence that is SEQ ID NO: 51 (mAb-GD2 VH) and (ii) a VL region wherein the VL region comprises a VL sequence that is SEQ ID NO: 52 (mAb-GD2 VL). In some embodiments, such VH and VL regions were used to construct bispecific binding agents (e.g., antibodies) each with a first binding domain that bind to GD2, including wherein the first binding domain comprises the mAb-GD2 VH and mAb-GD2 VL regions.

In some embodiments, such VH and VL regions were used to construct bispecific antibodies comprising four polypeptide chains, wherein (i) polypeptide chain 1 comprises a VL region (e.g., mAb-B7H3, Table 1), polypeptide chain 2 comprises a VH region (e.g., mAb-B7H3, Table 1), wherein the VL and VH regions form a first binding domain that binds to B7H3, and (ii) polypeptide chain 3 comprises a VL region (e.g., mAb-GD2, Table 2), polypeptide chain 4 comprises a VH region (e.g., mAb-GD2, Table 2), wherein the VL and VH regions form a second binding domain that binds to GD2.

In some embodiments, such VH and VL regions were used to construct bispecific antibodies comprising four polypeptide chains, wherein (i) polypeptide chain 1 comprises a VL region (e.g., mAb-GD2, Table 2), polypeptide chain 2 comprises a VH region (e.g., mAb-GD2, Table 2), wherein the VL and VH regions form a first binding domain that binds to GD2, and (ii) polypeptide chain 3 comprises a VL region (e.g., mAb-B7H3, Table 1), polypeptide chain 4 comprises a VH region (e.g., mAb-B7H3, Table 1), wherein the VL and VH regions form a second binding domain that binds to B7H3.

In a first exemplary embodiment of a bispecific binding agent (e.g., an antibody, such as a bispecific antibody), the first polypeptide chain has the amino acid sequence of sequence of SEQ ID NO: 53, the second polypeptide chain has the amino acid sequence of sequence of SEQ ID NO: 54, the third polypeptide chain has the amino acid sequence of sequence of SEQ ID NO: 55, and the fourth polypeptide chain has the amino acid sequence of sequence of SEQ ID NO: 56. In this embodiment, the first polypeptide chain and second polypeptide chain comprise an antigen binding domain for B7H3 and the third polypeptide chain and the fourth polypeptide chain comprise an antigen binding domain for GD2.

In a second exemplary embodiment of a bispecific binding agent (e.g., an antibody, such as a bispecific antibody), the first polypeptide chain has the amino acid sequence of sequence of SEQ ID NO: 57, the second polypeptide chain has the amino acid sequence of sequence of SEQ ID NO: 58, the third polypeptide chain has the amino acid sequence of sequence of SEQ ID NO: 59, and the fourth polypeptide chain has the amino acid sequence of sequence of SEQ ID NO: 60. In this embodiment, the first polypeptide chain and second polypeptide chain comprise an antigen binding domain for GD2 and the third polypeptide chain and the fourth polypeptide chain comprise an antigen binding domain for B7H3.

TABLE 1
mAb-B7H3 Variable Region Sequences

| | | Exemplary | IMGT | Kabat | Chothia | Contact | AbM |
|---|---|---|---|---|---|---|---|
| VH CDR | VH | GFTFSTYYIH | GFTFSTYY | TYYIH | GFTFSTY | STYYIH | GFTFSTYYIH |
| Seq. | CDR1 | (SEQ ID NO: 1) | (SEQ ID NO: 7) | (SEQ ID NO: 12) | (SEQ ID NO: 13) | (SEQ ID NO: 18) | (SEQ ID NO: 1) |
| | VH | WIHPSGKYTYYA | IHPSGKYT | WIHPSGKYTYYA | PSGK | WVAWIHPSGKYT | WIHPSGKYTY |
| | CDR2 | DSVKG | (SEQ ID NO: 8) | DSVKG | (SEQ ID NO: 14) | Y | (SEQ ID NO: 24) |
| | | (SEQ ID NO: 2) | | (SEQ ID NO: 2) | | (SEQ ID NO: 19) | |
| | VH | HYQVGAMDY | ARHYQVGAMDY | HYQVGAMDY | YQVGAMD | ARHYQVGAMD | HYQVGAMDY |
| | CDR3 | (SEQ ID NO: 3) | (SEQ ID NO: 9) | (SEQ ID NO: 3) | (SEQ ID NO: 15) | (SEQ ID NO: 20) | (SEQ ID NO: 3) |
| VL CDR | VL | RASQSVSSAVA | QSVSSA | RASQSVSSAVA | SQSVSSA | SSAVAWY | RASQSVSSAVA |
| Seq. | CDR1 | (SEQ ID NO: 4) | (SEQ ID NO: 10) | (SEQ ID NO: 4) | (SEQ ID NO: 16) | (SEQ ID NO: 21) | (SEQ ID NO: 4) |
| | VL | SASSLYS | SAS | SASSLYS | SAS | LLIYSASSLY | SASSLYS |
| | CDR2 | (SEQ ID NO: 5) | (SEQ ID NO: 11) | (SEQ ID NO: 5) | (SEQ ID NO: 11) | (SEQ ID NO: 22) | (SEQ ID NO: 5) |
| | VL | QQSGRSLYT | QQSGRSLYT | QQSGRSLYT | SGRSLY | QQSGRSLY | QQSGRSLYT |
| | CDR3 | (SEQ ID NO: 6) | (SEQ ID NO: 6) | (SEQ ID NO: 6) | (SEQ ID NO: 17) | (SEQ ID NO: 23) | (SEQ ID NO: 6) |

VH Sequence:
EVQLVESGGGLVQPGGSLRLSCAASGFTFSTYYIHWVRQAPGKGLEWVAWIHPSGKYTYYADSVKGRFTISADTSKNTAYLQMNSLRAEDTAVY
YCARHYQVGAMDYWGQGTLVTVSS (SEQ ID NO: 25)
VL Sequence:
DIQMTQSPSSLSASVGDRVTITCRASQSVSSAVAWYQQKPGKAPKLLIYSASSLYSGVPSRFSGSRSGTDFTLTISSLQPEDFATYYCQQSGRSLY
TFGQGTKVEIK (SEQ ID NO: 26)

TABLE 2
mAb-GD2 Variable Region Sequences

| | | Exemplary | IMGT | Kabat | Chothia | Contact | AbM |
|---|---|---|---|---|---|---|---|
| VH CDR | VH | GSSFTGYNMN | GSSFTGYN | GYNMN | GSSFTGY | TGYNMN | GSSFTGYNMN |
| Seq. | CDR1 | (SEQ ID NO: 27) | (SEQ ID NO: 33) | (SEQ ID NO: 38) | (SEQ ID NO: 39) | (SEQ ID NO: 44) | (SEQ ID NO: 27) |
| | VH | AIDPYYGGTYYA | IDPYYGGT | AIDPYYGGTYYA | PYYG | WAAIDPYYGGT | AIDPYYGGTY |
| | CDR2 | DSVKG | (SEQ ID NO: 34) | DSVKG | (SEQ ID NO: 40) | Y | (SEQ ID NO: 50) |
| | | (SEQ ID NO: 28) | | (SEQ ID NO: 28) | | (SEQ ID NO: 45) | |
| | VH | GMEY | ASGMEY | GMEY | ME | ASGME | GMEY |
| | CDR3 | (SEQ ID NO: 29) | (SEQ ID NO: 35) | (SEQ ID NO: 29) | (SEQ ID NO: 41) | (SEQ ID NO: 46) | (SEQ ID NO: 29) |
| VL CDR | VL | RSSQSLVHRNG | QSLVHRNGNTY | RSSQSLVHRNG | SQSLVHRNGNTY | VHRNGNTYLHW | RSSQSLVHRNG |
| Seq. | CDR1 | NTYLH | (SEQ ID NO: 36) | NTYLH | (SEQ ID NO: 42) | Y | NTYLH |
| | | (SEQ ID NO: 30) | | (SEQ ID NO: 30) | | (SEQ ID NO: 47) | (SEQ ID NO: 30) |
| | VL | KVSNRFS | KVS | KVSNRFS | KVS | LLIHKVSNRF | KVSNRFS |
| | CDR2 | (SEQ ID NO: 31) | (SEQ ID NO: 37) | (SEQ ID NO: 31) | (SEQ ID NO: 37) | (SEQ ID NO: 48) | (SEQ ID NO: 31) |
| | VL | SQSTHVPPLT | SQSTHVPPLT | SQSTHVPPLT | STHVPPL | SQSTHVPPL | SQSTHVPPLT |
| | CDR3 | (SEQ ID NO: 32) | (SEQ ID NO: 32) | (SEQ ID NO: 32) | (SEQ ID NO: 43) | (SEQ ID NO: 49) | (SEQ ID NO: 32) |

VH Sequence:
EVQLVESGGGLVQPGGSLRLSCAASGSSFTGYNMNWVRQAPGKGLEWVAAIDPYYGGTYYADSVKGRFTISADTSKNTAYLQMNSLRAEDTAV
YYCASGMEYWGQGTLVTVSS (SEQ ID NO: 51)
VL Sequence:
DIQMTQSPSSLSASVGDRVTITCRSSQSLVHRNGNTYLHWYQQKPGKAPKLLIHKVSNRFSGVPSRFSGSGSGTDFTLTISSLQPEDFATYYCSQS
THVPPLTFGQGTKVEIK (SEQ ID NO: 52)

In some embodiments, multispecific binding agents (e.g., antibodies, such as bispecific antibodies) described herein comprise a first binding domain that binds to B7H3, including human B7H3, and a second binding domain that binds to GD2, including human GD2, wherein the first binding domain and/or the second binding domain comprise a VH region. In other embodiments, multispecific binding agents (e.g., antibodies, such as bispecific antibodies) described herein comprise a first binding domain that binds to B7H3, including human B7H3, and a second binding domain that binds to GD2, including human GD2, wherein the first binding domain and/or the second binding domain comprise a VL region. In some embodiments, multispecific binding agents (e.g., antibodies, such as bispecific antibodies) described herein comprise a first binding domain that binds to B7H3, including human B7H3, and a second binding domain that binds GD2, including human GD2, wherein the first binding domain and/or the second binding domain have a combination of (i) a VH region; and (ii) a VL region.

In some embodiments, multispecific binding agents (e.g., antibodies, such as bispecific antibodies) described herein have a combination of: (i) a VH region as described herein; and (ii) a VL region as described herein, wherein the VH region is connected to a constant region as described herein and the VL region is connected to constant regions as described herein. For example, an exemplary multispecific binding agent comprises (i) a polypeptide chain having a combination of a VL region as described herein; and one or more constant regions (e.g., a CH2 and two CH3s or a CL, CH2 and CH3), and (ii) a polypeptide chain having a combination of a VH region as described herein and a constant region (CH3 or CH1). Such constant regions, for the various polypeptide chains described herein are depicted in FIG. 1 and described herein. For example, an exemplary first polypeptide chain described herein comprises any VL region as described herein and the following sequence having constant regions:

(SEQ ID NO: 69)
GQPREPQVCTLPPSRDELTKNQVSLTCLVRGFYPSDIAVEWESNGQPE
NNYKTTPPVLDSDGSFFLYSKLTVDKSRWQQGNVFSCSVMHEALHNHY
TQKSLSLSPGKDKTHTCPPCPAPELLGGPSVFLFPPKPKDTLMISRTP
EVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPP
SRDELTKNQVSLWCLVKGFYPSDIAVEWESNGQPENNYKTTPPVLDSD
GSFFLYSKLTVDKSRWQQGNVFSCSVMHEALHNHYTQKSLSLSPGK.

An exemplary third polypeptide chain as described herein comprises any VL region as described herein and the following sequence having constant regions:

(SEQ ID NO: 71)
RTVAAPSVFIFPPSDEQLKSGTASVVCLLNNFYPREAKVQWKVDNALQ
SGNSQESVTEQDSKDSTYSLSSTLTLSKADYEKHKVYACEVTHQGLSS
PVTKSFNRGECDKTHTCPPCPAPELLGGPSVFLFPPKPKDTLMISRTP
EVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPP
SREEMTKNQVSLSCAVKGFYPSDIAVEWESNGQPENNYKTTPPVLDSDG
SFFLVSKLTVDKSRWQQGNVFSCSVMHEALHNHYTQKSLSLSPGK.

An exemplary second polypeptide chain (e.g., for pairing with a first polypeptide chain) comprises any VH region as described herein and the following amino acid sequence having a constant region:

(SEQ ID NO: 70)
AQPREPQVYTLPPCRDWLTKNQVSLTCLVKGFYPSDIAVEWESNGQPE
NNYKTTPPVLDSDGSFFLYSKLTVDKSRWQQGNVFSCSVMHEALHNHY
TQKSLSLSPGK.

An exemplary fourth polypeptide chain (e.g., for pairing with a third polypeptide chain) comprises any VH region as described herein and the following amino acid sequence having a constant region:

(SEQ ID NO: 72)
ASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDK
KVEPKSC.

In some embodiments, multispecific binding agents (e.g., antibodies, such as bispecific antibodies) described herein have a first binding domain that binds to B7H3, including human B7H3, and second binding domain that binds GD2, including human GD2, wherein the first binding domain for binding to B7H3 comprises: (i) a VH region as described herein; and (ii) a VL region as described herein, wherein the second binding domain for binding to GD2 comprises: (i) a VH region as described herein; and (ii) a VL region as described herein, and wherein each of the VH regions for binding to B7H3 and GD2 are connected to a constant region as described herein (e.g., see FIG. 1) and the VL region is connected to constant regions as described herein (e.g., see FIG. 1). For example, in some embodiments, a multispecific binding agent (e.g., an antibody, such as bispecific antibody) comprises a first polypeptide chain and a second polypeptide chain that comprise a binding domain that binds to B7H3, including human B7H3, and a third polypeptide chain and a fourth polypeptide chain that comprise a binding domain that binds to GD2, including human GD2, wherein the first polypeptide chain comprises a combination of a VL region as described herein for binding to B7H3 and one or more contant regions as described herein (e.g., a CH2 and two variant CH3s), the second polypeptide chain comprises a combination of a VH region as described herein for binding to B7H3 and a constant region as described herein (e.g., variant CH3), the third polypeptide chain comprises a combination of a VL region as described herein for binding to GD2 and one or more contant regions as described herein (e.g., a CL, CH2 and CH3), and the fourth polypeptide chain comprises a combination of a VH region as described herein for binding to GD2 and a constant region as described herein (e.g., variant CH3). As another example, in some embodiments, a multispecific binding agent (e.g., an antibody, such as bispecific antibody) comprises a first polypeptide chain and a second polypeptide chain that comprise a binding domain that binds to GD2, including human GD2, and a third polypeptide chain and a fourth polypeptide chain that comprise a binding domain that binds to B7H3, including human B7H3, wherein the first polypeptide chain comprises a combination of a VL region as described herein for binding to GD2 and one or more contant regions as described herein (e.g., a CH2 and two variant CH3s), the second polypeptide chain comprises a combination of a VH region as described herein for binding to GD2 and a constant region as described herein (e.g., variant CH3), the third polypeptide chain comprises a combination of a VL region as described herein for binding to B7H3 and one or more contant regions as described herein (e.g., a CL, CH2 and CH3), and the fourth polypeptide chain comprises a combination of a VH region as described herein for binding to B7H3 and a constant region as described herein (e.g., variant CH3). In some embodiments, the constant regions for the first polypeptide chain comprise the amino acid sequence of SEQ ID NO: 69. In some embodiments, the constant region for the second polypeptide chain comprises the amino acid sequence of SEQ ID NO: 70. In some embodiments, the constant regions for the third polypeptide chain comprise the amino acid sequence of SEQ ID NO: 71. In some embodiments, the constant region for the fourth polypeptide chain comprises the amino acid sequence of SEQ ID NO: 72.

In some embodiments, multispecific binding agents (e.g., antibodies, such as bispecific antibodies) described herein comprise a first binding domain that binds to B7H3, including human B7H3, and second binding domain that binds GD2, including human GD2, wherein the first binding domain comprises one or more CDRs, including six CDRs, for example, VH CDR1, VH CDR2, VH CDR3, VL CDR1, VL CDR2, and/or VL CDR3 identified in Table 1, and wherein the second binding domain comprises one or more CDRs, including six CDRs, for example, VH CDR1, VH CDR2, VH CDR3, VL CDR1, VL CDR2, and/or VL CDR3 identified in Table 2. For example, in some embodiments, multispecific binding agents (e.g., antibodies, such as bispecific antibodies) described herein comprise a first polypeptide chain, a second polypeptide chain, a third polypeptide chain, and a fourth polypeptide chain, wherein: (A) the first polypeptide chain comprises a VL region and constant regions comprising the amino acid sequence of SEQ ID NO: 69, wherein the VL region comprises a VL CDR1, a VL CDR2, and a VL CDR3 amino acid sequence set forth in Table 1; (B) the second polypeptide chain comprises a VH region and a constant region comprising the amino acid sequence of SEQ ID NO: 70, wherein the VH region comprises a VH CDR1, a VH CDR2, and a VH CDR3 amino acid sequence set forth in Table 1; (C) the third polypeptide chain comprises a VL region and constant regions comprising the amino acid sequence of SEQ ID NO: 71, wherein the VL region comprises a VL CDR1, a VL CDR2, and a VL CDR3 amino acid sequence set forth in Table 2; and (D) the fourth polypeptide chain comprises a VH region and a constant region comprising the amino acid sequence of SEQ ID NO: 72, wherein the VH region comprises a VH CDR1, a VH CDR2, and a VH CDR3 amino acid sequence set forth in Table 2. As another example, in some embodiments, multispecific binding agents (e.g., antibodies, such as bispecific antibodies) described herein comprise a first polypeptide chain, a second polypeptide chain, a third polypeptide chain, and a fourth polypeptide chain, wherein: (A) the first polypeptide chain comprises a VL region and constant regions comprising the amino acid sequence of SEQ ID NO: 69, wherein the VL region comprises a VL CDR1, a VL CDR2, and a VL CDR3 amino acid sequence set forth in Table 2; (B) the second polypeptide chain comprises a VH region and a constant region comprising the amino acid sequence of SEQ ID NO: 70, wherein the VH region comprises a VH CDR1, a VH CDR2, and a VH CDR3 amino acid sequence set forth in Table 2; (C) the third polypeptide chain comprises a VL region and constant regions comprising the amino acid sequence of SEQ ID NO: 71, wherein the VL region comprises a VL CDR1, a VL CDR2, and a VL CDR3 amino acid sequence set forth in Table 1; and (D) the fourth polypeptide chain comprises a VH region and a constant region comprising the amino acid sequence of SEQ ID NO: 72, wherein the VH region comprises a VH CDR1, a VH CDR2, and a VH CDR3 amino acid sequence set forth in Table 1. As yet another example, in some embodiments, multispecific binding agents (e.g., antibodies, such as bispecific antibodies) described herein comprise a first polypeptide chain, a second polypeptide chain, a third polypeptide chain, and a fourth polypeptide chain, wherein: (A) the first polypeptide chain comprises a VL region and constant regions comprising the amino acid sequence of SEQ ID NO: 69, wherein the VL region comprises a VL CDR1 (e.g., SEQ ID NO: 4, 10, 16, or 21), a VL CDR2 (e.g., SEQ ID NO: 5, 11, or 22), and a VL CDR3 (e.g., SEQ ID NO: 6, 17, or 23) amino acid sequence; (B) the second polypeptide chain comprises a VH region and a constant region comprising the amino acid sequence of SEQ ID NO: 70, wherein the VH region comprises a VH CDR1 (e.g., SEQ ID NO: 1, 7, 12, 13, or 18), a VH CDR2 (e.g., SEQ ID NO: 2, 8, 14, 19, or 24), and a VH CDR3 (e.g., SEQ ID NO: 3, 9, 15, or 20) amino acid sequence; (C) the third polypeptide chain comprises a VL region and constant regions comprising the amino acid sequence of SEQ ID NO: 71, wherein the VL region comprises a VL CDR1 (e.g., SEQ ID NO: 30, 36, 42, or 47), a VL CDR2 (e.g., SEQ ID NO: 31, 37, or 48), and a VL CDR3 (e.g., SEQ ID NO: 32, 43, or 49) amino acid sequence; and (D) the fourth polypeptide chain comprises a VH region and a constant region comprising the amino acid sequence of SEQ ID NO: 72, wherein the VH region comprises a VH CDR1 (e.g., SEQ ID NO: 27, 33, 38, 39, or 44), a VH CDR2 (e.g., SEQ ID NO: 28, 34, 40, 45, or 50), and a VH CDR3 (e.g., SEQ ID NO: 29, 35, 41, or 46) amino acid sequence. As yet still another example, in some embodiments, multispecific binding agents (e.g., antibodies, such as bispecific antibodies) described herein comprise a first polypeptide chain, a second polypeptide chain, a third polypeptide chain, and a fourth polypeptide chain, wherein: (A) the first polypeptide chain comprises a VL region and constant regions comprising the amino acid sequence of SEQ ID NO: 69, wherein the VL region comprises a VL CDR1 (e.g., SEQ ID NO: 30, 36, 42, or 47), a VL CDR2 (e.g., SEQ ID NO: 31, 37, or 48), and a VL CDR3 (e.g., SEQ ID NO: 32, 43, or 49) amino acid sequence; (B) the second polypeptide chain comprises a VH region and a constant region comprising the amino acid sequence of SEQ ID NO: 70, wherein the VH region comprises a VH CDR1 (e.g., SEQ ID NO: 27, 33, 38, 39, or 44), a VH CDR2 (e.g., SEQ ID NO: 28, 34, 40, 45, or 50), and a VH CDR3 (e.g., SEQ ID NO: 29, 35, 41, or 46) amino acid sequence; (C) the third polypeptide chain comprises a VL region and constant regions comprising the amino acid sequence of SEQ ID NO: 71, wherein the VL region comprises a VL CDR1 (e.g., SEQ ID NO: 4, 10, 16, or 21), a VL CDR2 (e.g., SEQ ID NO: 5, 11, or 22), and a VL CDR3 (e.g., SEQ ID NO: 6,17, or 23) amino acid sequence; and (D) the fourth polypeptide chain comprises a VH region and a constant region comprising the amino acid sequence of SEQ ID NO: 72, wherein the VH region comprises a VH CDR1 (e.g., SEQ ID NO: 1, 7, 12, 13, or 18), a VH CDR2 (e.g., SEQ ID NO: 2, 8, 14, 19, or 24), and a VH CDR3 (e.g., SEQ ID NO: 3, 9, 15, or 20) amino acid sequence.

In some embodiments, multispecific binding agents (e.g., antibodies, such as bispecific antibodies), including multispecific binding agent that binds to B7H3, including human B7H3, and GD2, including human GD2, described herein comprise a first binding domain comprising one or more CDRs, including three VH CDRs, for example, VH CDR1, VH CDR2, VH CDR3, listed in Table 1. In other embodiments, multispecific binding agents (e.g., antibodies, such as bispecific antibodies), including multispecific binding agents that bind to B7H3, including human B7H3, and GD2, including human GD2, described herein comprise a first binding domain comprising one or more CDRs, including three CDRs, for example, VL CDR1, VL CDR2, and/or VL CDR3, listed in Table 1. In yet other embodiments, multispecific binding agents (e.g., antibodies, such as bispecific antibodies), including multispecific binding agents that bind to B7H3, including human B7H3, and GD2, including human GD2, described herein comprise a first binding domain comprising one or more CDRs, including three VH CDRs, for example, VH CDR1, VH CDR2, VH CDR3, listed in Table 1 and one or more CDRs, including three VL CDRs, for example, VL CDR1, VL CDR2, and/or VL CDR3, listed in Table 1.

In some embodiments, multispecific binding agents (e.g., antibodies, such as bispecific antibodies), including multispecific binding agent that binds to B7H3, including human B7H3, and GD2, including human GD2, described herein comprise a second binding domain comprising one or more CDRs, including three VH CDRs, for example, VH CDR1, VH CDR2, VH CDR3, listed in Table 2. In other embodiments, multispecific binding agents (e.g., antibodies, such as bispecific antibodies), including multispecific binding agents that bind to B7H3, including human B7H3, and GD2, including human GD2, described herein comprise a second binding domain comprising one or more CDRs, including three CDRs, for example, VL CDR1, VL CDR2, and/or VL CDR3, listed in Table 2. In yet other embodiments, multispecific binding agents (e.g., antibodies, such as bispecific antibodies), including multispecific binding agents that bind to B7H3, including human B7H3, and GD2, including human GD2, described herein comprise a second binding domain comprising one or more CDRs, including three VH CDRs, for example, VH CDR1, VH CDR2, VH CDR3, listed in Table 2 and one or more CDRs, including three VL CDRs, for example, VL CDR1, VL CDR2, and/or VL CDR3, listed in Table 2.

In some embodiments, a multispecific binding agent (e.g., an antibody, such as a bispecific antibody) described herein comprises a first binding domain that binds to B7H3 and comprises one or more complementarity determining regions (CDRs) comprising an amino acid sequence selected from a group consisting of SEQ ID NOS: 1-24. In some embodiments, a multispecific binding agent (e.g., an antibody, such as a bispecific antibody) described herein comprises a first binding domain that binds to B7H3 and comprises two or more complementarity determining regions (CDRs) comprising an amino acid sequence selected from a group consisting of SEQ ID NOS: 1-24. In some embodiments, a multispecific binding agent (e.g., an antibody, such as a bispecific antibody) described herein comprises a first binding domain that binds to B7H3 and comprises three or more complementarity determining regions (CDRs) comprising an amino acid sequence selected from a group consisting of SEQ ID NOS: 1-24. In some embodiments, the multispecific binding agent (e.g., an antibody, such as a bispecific antibody) described herein comprises a second binding domain that binds to GD2 and comprises one or more complementarity determining regions (CDRs) comprising an amino acid sequence selected from a group consisting of SEQ ID NOS: 27-50. In some embodiments, a multispecific binding agent (e.g., an antibody, such as a bispecific antibody) described herein comprises a second binding domain that binds to GD2 and comprises two or more complementarity determining regions (CDRs) comprising an amino acid sequence selected from a group consisting of SEQ ID NOS: 27-50. In some embodiments, a multispecific binding agent (e.g., an antibody, such as a bispecific antibody) described herein comprises a second binding domain that binds to GD2 and comprises three or more complementarity determining regions (CDRs) comprising an amino acid sequence selected from a group consisting of SEQ ID NOS: 27-50.

In some embodiments, a multispecific binding agent (e.g., an antibody, such as a bispecific antibody) described herein comprises a first binding domain that binds to B7H3 and comprises a VH with one or more (e.g., one, two or three) VH CDRs listed in Table 1. In other embodiments, a multispecific binding agent (e.g., an antibody, such as a bispecific antibody) described herein comprises a first binding domain that binds to B7H3 and comprises a VL with one or more (e.g., one, two or three) VL CDRs listed in Table 1. In yet other embodiments, the multispecific binding agent (e.g., an antibody, such as a bispecific antibody) described herein comprises a first binding domain that binds to B7H3 and comprises one or more (e.g., one, two or three) VH CDRs listed in Table 1 and one or more VL CDRs listed in Table 1. Accordingly, in some embodiments, a multispecific binding agent (e.g., an antibody, such as a bispecific antibody) described herein comprises a first binding domain that binds to B7H3 and comprises a VH CDR1 having the amino acid sequence of any one of SEQ ID NOS: 1, 7, 12, 13, and 18. In some embodiments, a multispecific binding agent (e.g., an antibody, such as a bispecific antibody) described herein comprises a first binding domain that binds to B7H3 and comprises a VH CDR2 having the amino acid sequence of any one of SEQ ID NOS: 2, 8, 14, 19, and 24. In some embodiments, a multispecific binding agent (e.g., an antibody, such as a bispecific antibody) described herein a first binding domain that binds to B7H3 and comprises a VH CDR3 having the amino acid sequence of any one of SEQ ID NOS: 3, 9, 15, and 20. In some embodiments, a multispecific binding agent (e.g., an antibody, such as a bispecific antibody) described herein comprises a first binding domain that binds to B7H3 and comprises a VH CDR1 and/or a VH CDR2 and/or a VH CDR3 independently selected from a VH CDR1, VH CDR2, VH CDR3 as depicted in any one of the amino acid sequences depicted in Table 1. In some embodiments, a multispecific binding agent (e.g., an antibody, such as a bispecific antibody) described herein comprises a first binding domain that binds to B7H3 and comprises a VL CDR1 having the amino acid sequence of any one of SEQ ID NOS: 4, 10, 16, and 21. In another embodiment, a multispecific binding agent (e.g., an antibody, such as a bispecific antibody) described herein comprises a first binding domain that binds to B7H3 and comprises a VL CDR2 having the amino acid sequence of any one of SEQ ID NOS: 5, 11, and 22. In some embodiments, a multispecific binding agent (e.g., an antibody, such as a bispecific antibody) described herein comprises a first binding domain that binds to B7H3 and comprises a VL CDR3 having the amino acid sequence of any one of SEQ ID NOS: 6, 17, and 23. In some embodiments, a multispecific binding agent (e.g., an antibody, such as a bispecific antibody) described herein comprises a first binding domain that binds to B7H3 and comprises a VL CDR1 and/or a VL CDR2 and/or a VL CDR3 independently selected from a VL CDR1, VL CDR2, VL CDR3 as depicted in any one of the amino acid sequences depicted in Table 1.

In some embodiments, a multispecific binding agent (e.g., an antibody, such as a bispecific antibody) described herein comprises a second binding domain that binds to GD2 and comprises a VH with one or more (e.g., one, two or three) VH CDRs listed in Table 2. In other embodiments, a multispecific binding agent (e.g., an antibody, such as a bispecific antibody) described herein comprises a second binding domain that binds to GD2 and comprises a VL with one or more (e.g., one, two or three) VL CDRs listed in Table 2. In yet other embodiments, the multispecific binding agent (e.g., an antibody, such as a bispecific antibody) described herein comprises a second binding domain that binds to GD2 and comprises one or more (e.g., one, two or three) VH CDRs listed in Table 2 and one or more VL CDRs listed in Table 2. Accordingly, in some embodiments, a multispecific binding agent (e.g., an antibody, such as a bispecific antibody) described herein comprises a second binding domain that binds to GD2 and comprises a VH CDR1 having the amino acid sequence of any one of SEQ ID NOS: 27, 33, 38, 39, and 44. In some embodiments, a multispecific binding agent (e.g., an antibody, such as a bispecific antibody) described herein comprises a second binding domain that binds to GD2 and comprises a VH CDR2 having the amino acid sequence of any one of SEQ ID NOS: 28, 34, 40, 45, and 50. In some embodiments, a multispecific binding agent (e.g., an antibody, such as a bispecific antibody) described herein a second binding domain that binds to GD2 and comprises a VH CDR3 having the amino acid sequence of any one of SEQ ID NOS: 29, 35, 41, and 46. In some embodiments, a multispecific binding agent (e.g., an antibody, such as a bispecific antibody) described herein comprises a second binding domain that binds to GD2 and comprises a VH CDR1 and/or a VH CDR2 and/or a VH CDR3 independently selected from a VH CDR1, VH CDR2, VH CDR3 as depicted in any one of the amino acid sequences depicted in Table 2. In some embodiments, a multispecific binding agent (e.g., an antibody, such as a bispecific antibody) described herein comprises a second binding domain that binds to GD2 and comprises a VL CDR1 having the amino acid sequence of any one of SEQ ID NOS: 30, 36, 42, and 47. In another embodiment, a multispecific binding agent (e.g., an antibody, such as a bispecific antibody) described herein comprises a second binding domain that binds to GD2 and comprises a VL CDR2 having the amino acid sequence of any one of SEQ ID NOS: 31, 37, and 48. In some embodiments, a multispecific binding agent (e.g., an antibody, such as a bispecific antibody) described herein comprises a second binding domain that binds to GD2 and comprises a VL CDR3 having the amino acid sequence of any one of SEQ ID NOS: 32, 43, and 49. In some embodiments, a multispecific binding agent (e.g., an antibody, such as a bispecific antibody) described herein comprises a second binding domain that binds to GD2 and comprises a VL CDR1 and/or a VL CDR2 and/or a VL CDR3 independently selected from a VL CDR1, VL CDR2, VL CDR3 as depicted in any one of the amino acid sequences depicted in Table 2.

In some embodiments, a multispecific binding agent (e.g., an antibody, such as a bispecific antibody) described herein comprises a first binding domain that binds to B7H3 and comprises a VH region comprising: (1) a VH CDR1 having an amino acid sequence of selected from the group consisting of: (i) SEQ ID NO:1, (ii) SEQ ID NO:7, (iii) SEQ ID NO:12, (iv) SEQ ID NO:13, and (v) SEQ ID NO:18; (2) a VH CDR2 having an amino acid sequence of selected from the group consisting of: (i) SEQ ID NO:2, (ii) SEQ ID NO:8, (iii) SEQ ID NO:14, (iv) SEQ ID NO:19, and (v) SEQ ID NO:24; and (3) a VH CDR3 having an amino acid sequence of selected from the group consisting of: (i) SEQ ID NO:3, (ii) SEQ ID NO:9, (iii) SEQ ID NO:15, and (iv) SEQ ID NO:20; and/or a VL region comprising: (1) a VL CDR1 having an amino acid sequence of selected from the group consisting of: (i) SEQ ID NO:4, (ii) SEQ ID NO:10, (iii) SEQ ID NO:16, and (iv) SEQ ID NO:21; (2) a VL CDR2 having an amino acid sequence of selected from the group consisting of: (i) SEQ ID NO:5, (ii) SEQ ID NO:11, and (iii) SEQ ID NO:22; and (3) a VL CDR3 having an amino acid sequence of selected from the group consisting of: (i) SEQ ID NO:6, (ii) SEQ ID NO:17, and (iii) SEQ ID NO:23.

In some embodiments, a multispecific binding agent (e.g., an antibody, such as a bispecific antibody) described herein comprises a first binding domain that binds to B7H3 and comprises a VH region comprising: (1) a VH CDR1 having an amino acid sequence of selected from the group consisting of: (i) SEQ ID NO:1, (ii) SEQ ID NO:7, (iii) SEQ ID NO:12, (iv) SEQ ID NO:13, and (v) SEQ ID NO:18; (2) a VH CDR2 having an amino acid sequence of selected from the group consisting of: (i) SEQ ID NO:2, (ii) SEQ ID NO:8, (iii) SEQ ID NO:14, (iv) SEQ ID NO:19, and (v) SEQ ID NO:24; and (3) a VH CDR3 having an amino acid sequence of selected from the group consisting of: (i) SEQ ID NO:3, (ii) SEQ ID NO:9, (iii) SEQ ID NO:15, and (iv) SEQ ID NO:20.

In some embodiments, a multispecific binding agent (e.g., an antibody, such as a bispecific antibody) described herein comprises a first binding domain that binds to B7H3 and comprises a VL region comprising: (1) a VL CDR1 having an amino acid sequence of selected from the group consisting of: (i) SEQ ID NO:4, (ii) SEQ ID NO:10, (iii) SEQ ID NO:16, and (iv) SEQ ID NO:21; (2) a VL CDR2 having an amino acid sequence of selected from the group consisting of: (i) SEQ ID NO:5, (ii) SEQ ID NO:11, and (iii) SEQ ID NO:22; and (3) a VL CDR3 having an amino acid sequence of selected from the group consisting of: (i) SEQ ID NO:6, (ii) SEQ ID NO:17, and (iii) SEQ ID NO:23.

In some embodiments, a multispecific binding agent (e.g., an antibody, such as a bispecific antibody) described herein comprises a second binding domain that binds to GD2 and comprises (a) a VH region comprising (1) a VH CDR1 having an amino acid sequence selected from the group consisting of: (i) SEQ ID NO:27, (ii) SEQ ID NO:33, (iii) SEQ ID NO:38, (iv) SEQ ID NO:39 (v) SEQ ID NO:44; (2) a VH CDR2 having an amino acid sequence selected from the group consisting of: (i) SEQ ID NO:28, (ii) SEQ ID NO:34, (iii) SEQ ID NO:40, (iv) SEQ ID NO:45, (v) SEQ ID NO:50; and (3) a VH CDR3 having an amino acid sequence selected from the group consisting of: (i) SEQ ID NO:29, (ii) SEQ ID NO:35, (iii) SEQ ID NO:41, (iv) SEQ ID NO:46; and/or (b) a VL region comprising: (1) a VL CDR1 having an amino acid sequence selected from the group consisting of: (i) SEQ ID NO:30, (ii) SEQ ID NO:36, (iii) SEQ ID NO:42, (iv) SEQ ID NO:47; (2) a VL CDR2 having an amino acid sequence selected from the group consisting of: (i) SEQ ID NO:31, (ii) SEQ ID NO:37, (iii) SEQ ID NO:48; (3) a VL CDR3 having an amino acid sequence selected from the group consisting of: (i) SEQ ID NO:32, (ii) SEQ ID NO:43, (iii) SEQ ID NO:49.

In some embodiments, a multispecific binding agent (e.g., an antibody, such as a bispecific antibody) described herein comprises a second binding domain that binds to GD2 and comprises a VH region comprising (1) a VH CDR1 having an amino acid sequence selected from the group consisting of: (i) SEQ ID NO:27, (ii) SEQ ID NO:33, (iii) SEQ ID NO:38, (iv) SEQ ID NO:39 (v) SEQ ID NO:44; (2) a VH CDR2 having an amino acid sequence selected from the group consisting of: (i) SEQ ID NO:28, (ii) SEQ ID NO:34, (iii) SEQ ID NO:40, (iv) SEQ ID NO:45, (v) SEQ ID NO:50; and (3) a VH CDR3 having an amino acid sequence selected from the group consisting of: (i) SEQ ID NO:29, (ii) SEQ ID NO:35, (iii) SEQ ID NO:41, (iv) SEQ ID NO:47.

In some embodiments, a multispecific binding agent (e.g., an antibody, such as a bispecific antibody) described herein comprises a second binding domain that binds to GD2 and comprises a VL region comprising: (1) a VL CDR1 having an amino acid sequence selected from the group consisting of: (i) SEQ ID NO:30, (ii) SEQ ID NO:36, (iii) SEQ ID NO:42, (iv) SEQ ID NO:47; (2) a VL CDR2 having an amino acid sequence selected from the group consisting of: (i) SEQ ID NO:31, (ii) SEQ ID NO:37, (iii) SEQ ID NO:48; (3) a VL CDR3 having an amino acid sequence selected from the group consisting of: (i) SEQ ID NO:32, (ii) SEQ ID NO:43, (iii) SEQ ID NO:49.

Also described herein are multispecific binding agents (e.g., antibodies, such as bispecific antibodies) comprising a first binding domain that binds to B7H3 and comprises one or more (e.g., one, two or three) VH CDRs and one or more (e.g., one, two or three) VL CDRs listed in Table 1. In particular, described herein is a multispecific binding agent (e.g., an antibody, such as a bispecific antibody) comprising: a VH CDR1 (SEQ ID NOS:1, 7, 12, 13, and 18) and a VL CDR1 (SEQ ID NOS:4, 10, 16, and 21); a VH CDR1 (SEQ ID NOS:1, 7, 12, 13, and 18) and a VL CDR2 (SEQ ID NOS:5, 11, and 22); a VH CDR1 (SEQ ID NOS:1, 7, 12, 13, and 18) and a VL CDR3 (SEQ ID NOS:6, 17, and 23); a VH CDR2 (SEQ ID NOS:2, 8, 14, 19, and 24) and a VL CDR1 (SEQ ID NOS:4, 10, 16, and 21); a VH CDR2 (SEQ ID NOS:2, 8, 14, 19, and 24) and a VL CDR2 (SEQ ID NOS:5, 11, and 22); a VH CDR2 (SEQ ID NOS:2, 8, 14, 19, and 24) and a VL CDR3 (SEQ ID NOS:6, 17, and 23); a VH CDR3 (SEQ ID NOS:3, 9, 15, and 20) and a VL CDR1 (SEQ ID NOS:4, 10, 16, and 21); a VH CDR3 (SEQ ID NOS:3, 9, 15, and 20) and a VL CDR2 (SEQ ID NOS:5, 11, and 22); a VH CDR3 (SEQ ID NOS:3, 9, 15, and 20) and a VL CDR3 (SEQ ID NOS:6, 17, and 23); a VH CDR1 (SEQ ID NOS:1, 7, 12, 13, and 18), a VH CDR2 (SEQ ID NOS:2, 8, 14, 19, and 24) and a VL CDR1 (SEQ ID NOS:4, 10, 16, and 21); a VH CDR1 (SEQ ID NOS:1, 7, 12, 13, and 18), a VH CDR2 (SEQ ID NOS:2, 8, 14, 19, and 24) and a VL CDR2 (SEQ ID NOS:5, 11, and 22); a VH CDR1 (SEQ ID NOS:1, 7, 12, 13, and 18), a VH CDR2 (SEQ ID NOS:2, 8, 14, 19, and 24) and a VL CDR3 (SEQ ID NOS:6, 17, and 23); a VH CDR2 (SEQ ID NOS:2, 8, 14, 19, and 24), a VH CDR3 (SEQ ID NOS:3, 9, 15, and 20) and a VL CDR1 (SEQ ID NOS:4, 10, 16, and 21); a VH CDR2 (SEQ ID NOS:2, 8, 14, 19, and 24), a VH CDR3 (SEQ ID NOS:3, 9, 15, and 20) and a VL CDR2 (SEQ ID NOS:5, 11, and 22); a VH CDR2 (SEQ ID NOS:2, 8, 14, 19, and 24), a VH CDR3 (SEQ ID NOS:3, 9, 15, and 20) and a VL CDR3 (SEQ ID NOS:6, 17, and 23); a VH CDR1 (SEQ ID NOS:1, 7, 12, 13, and 18), a VL CDR1 (SEQ ID NOS:4, 10, 16, and 21) and a VL CDR2 (SEQ ID NOS:5, 11, and 22); a VH CDR1 (SEQ ID NOS:1, 7, 12, 13, and 18), a VL CDR1 (SEQ ID NOS:4, 10, 16, and 21) and a VL CDR3 (SEQ ID NOS:6, 17, and 23); a VH CDR1 (SEQ ID NOS:1, 7, 12, 13, and 18), a VL CDR2 (SEQ ID NOS:5, 11, and 22) and a VL CDR3 (SEQ ID NOS:6, 17, and 23); a VH CDR2 (SEQ ID NOS:2, 8, 14, 19, and 24), a VL CDR1 (SEQ ID NOS:4, 10, 16, and 21) and a VL CDR2 (SEQ ID NOS:5, 11, and 22); a VH CDR2 (SEQ ID NOS:2, 8, 14, 19, and 24), a VL CDR1 (SEQ ID NOS:4, 10, 16, and 21) and a VL CDR3 (SEQ ID NOS:6, 17, and 23); a VH CDR2 (SEQ ID NOS:2, 8, 14, 19, and 24), a VL CDR2 (SEQ ID NOS:5, 11, and 22) and a VL CDR3 (SEQ ID NOS:6, 17, and 23); a VH CDR3 (SEQ ID NOS:3, 9, 15, and 20), a VL CDR1 (SEQ ID NOS:4, 10, 16, and 21) and a VL CDR2 (SEQ ID NOS:5, 11, and 22); a VH CDR3 (SEQ ID NOS:3, 9, 15, and 20), a VL CDR1 (SEQ ID NOS:4, 10, 16, and 21) and a VL CDR3 (SEQ ID NOS:6, 17, and 23); a VH CDR3 (SEQ ID NOS:3, 9, 15, and 20), a VL CDR2 (SEQ ID NOS:5, 11, and 22) and a VL CDR3 (SEQ ID NOS:6, 17, and 23); a VH CDR1 (SEQ ID NOS:1, 7, 12, 13, and 18), a VH CDR2 (SEQ ID NOS:2, 8, 14, 19, and 24), a VH CDR3 (SEQ ID NOS:3, 9, 15, and 20) and a VL CDR1 (SEQ ID NOS:4, 10, 16, and 21); a VH CDR1 (SEQ ID NOS:1, 7, 12, 13, and 18), a VH CDR2 (SEQ ID NOS:2, 8, 14, 19, and 24), a VH CDR3 (SEQ ID NOS:3, 9, 15, and 20) and a VL CDR2 (SEQ ID NOS:5, 11, and 22); a VH CDR1 (SEQ ID NOS:1, 7, 12, 13, and 18), a VH CDR2 (SEQ ID NOS:2, 8, 14, 19, and 24), a VH CDR3 (SEQ ID NOS:3, 9, 15, and 20) and a VL CDR3 (SEQ ID NOS:6, 17, and 23); a VH CDR1 (SEQ ID NOS:1, 7, 12, 13, and 18), a VH CDR2 (SEQ ID NOS:2, 8, 14, 19, and 24), a VL CDR1 (SEQ ID NOS:4, 10, 16, and 21) and a VL CDR2 (SEQ ID NOS:5, 11, and 22); a VH CDR1 (SEQ ID NOS:1, 7, 12, 13, and 18), a VH CDR2 (SEQ ID NOS:2, 8, 14, 19, and 24), a VL CDR1 (SEQ ID NOS:4, 10, 16, and 21) and a VL CDR3 (SEQ ID NOS:6, 17, and 23); a VH CDR1 (SEQ ID NOS:1, 7, 12, 13, and 18), a VH CDR2 (SEQ ID NOS:2, 8, 14, 19, and 24), a VL CDR2 (SEQ ID NOS:5, 11, and 22) and a VL CDR3 (SEQ ID NOS:6, 17, and 23); a VH CDR1 (SEQ ID NOS:1, 7, 12, 13, and 18), a VH CDR3 (SEQ ID NOS:3, 9, 15, and 20), a VL CDR1 (SEQ ID NOS:4, 10, 16, and 21) and a VL CDR2 (SEQ ID NOS:5, 11, and 22); a VH CDR1 (SEQ ID NOS:1, 7, 12, 13, and 18), a VH CDR3 (SEQ ID NOS:3, 9, 15, and 20), a VL CDR1 (SEQ ID NOS:4, 10, 16, and 21) and a VL CDR3 (SEQ ID NOS:6, 17, and 23); a VH CDR1 (SEQ ID NOS:1, 7, 12, 13, and 18), a VH CDR3 (SEQ ID NOS:3, 9, 15, and 20), a VL CDR2 (SEQ ID NOS:5, 11, and 22) and a VL CDR3 (SEQ ID NOS:6, 17, and 23); a VH CDR2 (SEQ ID NOS:2, 8, 14, 19, and 24), a VH CDR3 (SEQ ID NOS:3, 9, 15, and 20), a VL CDR1 (SEQ ID NOS:4, 10, 16, and 21) and a VL CDR2 (SEQ ID NOS:5, 11, and 22); a VH CDR2 (SEQ ID NOS:2, 8, 14, 19, and 24), a VH CDR3 (SEQ ID NOS:3, 9, 15, and 20), a VL CDR1 (SEQ ID NOS:4, 10, 16, and 21) and a VL CDR3 (SEQ ID NOS:6, 17, and 23); a VH CDR2 (SEQ ID NOS:2, 8, 14, 19, and 24), a VH CDR3 (SEQ ID NOS:3, 9, 15, and 20), a VL CDR2 (SEQ ID NOS:5, 11, and 22) and a VL CDR3 (SEQ ID NOS:6, 17, and 23); a VH CDR1 (SEQ ID NOS:1, 7, 12, 13, and 18), a VH CDR2 (SEQ ID NOS:2, 8, 14, 19, and 24), a VH CDR3 (SEQ ID NOS:3, 9, 15, and 20), a VL CDR1 (SEQ ID NOS:4, 10, 16, and 21) and a VL CDR2 (SEQ ID NOS:5, 11, and 22); a VH CDR1 (SEQ ID NOS:1, 7, 12, 13, and 18), a VH CDR2 (SEQ ID NOS:2, 8, 14, 19, and 24), a VH CDR3 (SEQ ID NOS:3, 9, 15, and 20), a VL CDR1 (SEQ ID NOS:4, 10, 16, and 21) and a VL CDR3 (SEQ ID NOS:6, 17, and 23); a VH CDR1 (SEQ ID NOS:1, 7, 12, 13, and 18), a VH CDR2 (SEQ ID NOS:2, 8, 14, 19, and 24), a VH CDR3 (SEQ ID NOS:3, 9, 15, and 20), a VL CDR2 (SEQ ID NOS:5, 11, and 22) and a VL CDR3 (SEQ ID NOS:6, 17, and 23); a VH CDR1 (SEQ ID NOS:1, 7, 12, 13, and 18), a VH CDR2 (SEQ ID NOS:2, 8, 14, 19, and 24), a VL CDR1 (SEQ ID NOS:4, 10, 16, and 21), a VL CDR2 (SEQ ID NOS:5, 11, and 22), and a VL CDR3 (SEQ ID NOS:6, 17, and 23); a VH CDR1 (SEQ ID NOS:1, 7, 12, 13, and 18), a VH CDR3 (SEQ ID NOS:3, 9, 15, and 20), a VL CDR1 (SEQ ID NOS:4, 10, 16, and 21), a VL CDR2 (SEQ ID NOS:5, 11, and 22), and a VL CDR3 (SEQ ID NOS:6, 17, and 23); a VH CDR2 (SEQ ID NOS:2, 8, 14, 19, and 24), a VH CDR3 (SEQ ID NOS:3, 9, 15, and 20), a VL CDR1 (SEQ ID NOS:4, 10, 16, and 21), a VL CDR2 (SEQ ID NOS:5, 11, and 22), and a VL CDR3 (SEQ ID NOS:6, 17, and 23); or any combination thereof of the VH CDRs (SEQ ID NOS:1, 2, 3, 7, 8, 9, 12, 13, 14, 15, 18, 19, 20, and 24) and VL CDRs (SEQ ID NOS:4, 5, 6, 10, 11, 16, 17, 21, 22, and 23) listed in Table 1.

Also described herein are multispecific binding agents (e.g., antibodies) comprising a second binding domain that binds to GD2 and comprises one or more (e.g., one, two or three) VH CDRs and one or more (e.g., one, two or three) VL CDRs listed in Table 2. In particular, described herein is a multispecific binding agent (e.g., an antibody, such as a bispecific antibody) comprising: a VH CDR1 (SEQ ID NOS:27, 33, 38, 39, and 44) and a VL CDR1 (SEQ ID NOS:30, 36, 42, and 47); a VH CDR1 (SEQ ID NOS:27, 33, 38, 39, and 44) and a VL CDR2 (SEQ ID NOS:31, 37, and 48); a VH CDR1 (SEQ ID NOS:27, 33, 38, 39, and 44) and a VL CDR3 (SEQ ID NOS:32, 43, and 49); a VH CDR2 (SEQ ID NOS:28, 34, 40, 45, and 50) and a VL CDR1 (SEQ ID NOS:30, 36, 42, and 47); a VH CDR2 (SEQ ID NOS:28, 34, 40, 45, and 50) and a VL CDR2 (SEQ ID NOS:31, 37, and 48); a VH CDR2 (SEQ ID NOS:28, 34, 40, 45, and 50) and a VL CDR3 (SEQ ID NOS:32, 43, and 49); a VH CDR3 (SEQ ID NOS:29, 35, 41, and 46) and a VL CDR1 (SEQ ID NOS:30, 36, 42, and 47); a VH CDR3 (SEQ ID NOS:29, 35, 41, and 46) and a VL CDR2 (SEQ ID NOS:31, 37, and 48); a VH CDR3 (SEQ ID NOS:29, 35, 41, and 46) and a VL CDR3 (SEQ ID NOS:32, 43, and 49); a VH CDR1 (SEQ ID NOS:27, 33, 38, 39, and 44), a VH CDR2 (SEQ ID NOS:28, 34, 40, 45, and 50) and a VL CDR1 (SEQ ID NOS:30, 36, 42, and 47); a VH CDR1 (SEQ ID NOS:27, 33, 38, 39, and 44), a VH CDR2 (SEQ ID NOS:28, 34, 40, 45, and 50) and a VL CDR2 (SEQ ID NOS:31, 37, and 48); a VH CDR1 (SEQ ID NOS:27, 33, 38, 39, and 44), a VH CDR2 (SEQ ID NOS:28, 34, 40, 45, and 50) and a VL CDR3 (SEQ ID NOS:32, 43, and 49); a VH CDR2 (SEQ ID NOS:28, 34, 40, 45, and 50), a VH CDR3 (SEQ ID NOS:29, 35, 41, and 46) and a VL CDR1 (SEQ ID NOS:30, 36, 42, and 47); a VH CDR2 (SEQ ID NOS:28, 34, 40, 45, and 50), a VH CDR3 (SEQ ID NOS:29, 35, 41, and 46) and a VL CDR2 (SEQ ID NOS:31, 37, and 48); a VH CDR2 (SEQ ID NOS:28, 34, 40, 45, and 50), a VH CDR3 (SEQ ID NOS:29, 35, 41, and 46) and a VL CDR3 (SEQ ID NOS:32, 43, and 49); a VH CDR1 (SEQ ID NOS:27, 33, 38, 39, and 44), a VL CDR1 (SEQ ID NOS:30, 36, 42, and 47) and a VL CDR2 (SEQ ID NOS:31, 37, and 48); a VH CDR1 (SEQ ID NOS:27, 33, 38, 39, and 44), a VL CDR1 (SEQ ID NOS:30, 36, 42, and 47) and a VL CDR3 (SEQ ID NOS:32, 43, and 49); a VH CDR1 (SEQ ID NOS:27, 33, 38, 39, and 44), a VL CDR2 (SEQ ID NOS:31, 37, and 48) and a VL CDR3 (SEQ ID NOS:32, 43, and 49); a VH CDR2 (SEQ ID NOS:28, 34, 40, 45, and 50), a VL CDR1 (SEQ ID NOS:30, 36, 42, and 47) and a VL CDR2 (SEQ ID NOS:31, 37, and 48); a VH CDR2 (SEQ ID NOS:28, 34, 40, 45, and 50), a VL CDR1 (SEQ ID NOS:30, 36, 42, and 47) and a VL CDR3 (SEQ ID NOS:32, 43, and 49); a VH CDR2 (SEQ ID NOS:28, 34, 40, 45, and 50), a VL CDR2 (SEQ ID NOS:31, 37, and 48) and a VL CDR3 (SEQ ID NOS:32, 43, and 49); a VH CDR3 (SEQ ID NOS:29, 35, 41, and 46), a VL CDR1 (SEQ ID NOS:30, 36, 42, and 47) and a VL CDR2 (SEQ ID NOS:31, 37, and 48); a VH CDR3 (SEQ ID NOS:29, 35, 41, and 46), a VL CDR1 (SEQ ID NOS:30, 36, 42, and 47) and a VL CDR3 (SEQ ID NOS:32, 43, and 49); a VH CDR3 (SEQ ID NOS:29, 35, 41, and 46), a VL CDR2 (SEQ ID NOS:31, 37, and 48) and a VL CDR3 (SEQ ID NOS:32, 43, and 49); a VH CDR1 (SEQ ID NOS:27, 33, 38, 39, and 44), a VH CDR2 (SEQ ID NOS:28, 34, 40, 45, and 50), a VH CDR3 (SEQ ID NOS:29, 35, 41, and 46) and a VL CDR1 (SEQ ID NOS:30, 36, 42, and 47); a VH CDR1 (SEQ ID NOS:27, 33, 38, 39, and 44), a VH CDR2 (SEQ ID NOS:28, 34, 40, 45, and 50), a VH CDR3 (SEQ ID NOS:29, 35, 41, and 46) and a VL CDR2 (SEQ ID NOS:31, 37, and 48); a VH CDR1 (SEQ ID NOS:27, 33, 38, 39, and 44), a VH CDR2 (SEQ ID NOS:28, 34, 40, 45, and 50), a VH CDR3 (SEQ ID NOS:29, 35, 41, and 46) and a VL CDR3 (SEQ ID NOS:32, 43, and 49); a VH CDR1 (SEQ ID NOS:27, 33, 38, 39, and 44), a VH CDR2 (SEQ ID NOS:28, 34, 40, 45, and 50), a VL CDR1 (SEQ ID NOS:30, 36, 42, and 47) and a VL CDR2 (SEQ ID NOS:31, 37, and 48); a VH CDR1 (SEQ ID NOS:27, 33, 38, 39, and 44), a VH CDR2 (SEQ ID NOS:28, 34, 40, 45, and 50), a VL CDR1 (SEQ ID NOS:30, 36, 42, and 47) and a VL CDR3 (SEQ ID NOS:32, 43, and 49); a VH CDR1 (SEQ ID NOS:27, 33, 38, 39, and 44), a VH CDR2 (SEQ ID NOS:28, 34, 40, 45, and 50), a VL CDR2 (SEQ ID NOS:31, 37, and 48) and a VL CDR3 (SEQ ID NOS:32, 43, and 49); a VH CDR1 (SEQ ID NOS:27, 33, 38, 39, and 44), a VH CDR3 (SEQ ID NOS:29, 35, 41, and 46), a VL CDR1 (SEQ ID NOS:30, 36, 42, and 47) and a VL CDR2 (SEQ ID NOS:31, 37, and 48); a VH CDR1 (SEQ ID NOS:27, 33, 38, 39, and 44), a VH CDR3 (SEQ ID NOS:29, 35, 41, and 46), a VL CDR1 (SEQ ID NOS:30, 36, 42, and 47) and a VL CDR3 (SEQ ID NOS:32, 43, and 49); a VH CDR1 (SEQ ID NOS:27, 33, 38, 39, and 44), a VH CDR3 (SEQ ID NOS:29, 35, 41, and 46), a VL CDR2 (SEQ ID NOS:31, 37, and 48) and a VL CDR3 (SEQ ID NOS:32, 43, and 49); a VH CDR2 (SEQ ID NOS:28, 34, 40, 45, and 50), a VH CDR3 (SEQ ID NOS:29, 35, 41, and 46), a VL CDR1 (SEQ ID NOS:30, 36, 42, and 47) and a VL CDR2 (SEQ ID NOS:31, 37, and 48); a VH CDR2 (SEQ ID NOS:28, 34, 40, 45, and 50), a VH CDR3 (SEQ ID NOS:29, 35, 41, and 46), a VL CDR1 (SEQ ID NOS:30, 36, 42, and 47) and a VL CDR3 (SEQ ID NOS:32, 43, and 49); a VH CDR2 (SEQ ID NOS:28, 34, 40, 45, and 50), a VH CDR3 (SEQ ID NOS:29, 35, 41, and 46), a VL CDR2 (SEQ ID NOS:31, 37, and 48) and a VL CDR3 (SEQ ID NOS:32, 43, and 49); a VH CDR1 (SEQ ID NOS:27, 33, 38, 39, and 44), a VH CDR2 (SEQ ID NOS:28, 34, 40, 45, and 50), a VH CDR3 (SEQ ID NOS:29, 35, 41, and 46), a VL CDR1 (SEQ ID NOS:30, 36, 42, and 47) and a VL CDR2 (SEQ ID NOS:31, 37, and 48); a VH CDR1 (SEQ ID NOS:27, 33, 38, 39, and 44), a VH CDR2 (SEQ ID NOS:28, 34, 40, 45, and 50), a VH CDR3 (SEQ ID NOS:29, 35, 41, and 46), a VL CDR1 (SEQ ID NOS:30, 36, 42, and 47) and a VL CDR3 (SEQ ID NOS:32, 43, and 49); a VH CDR1 (SEQ ID NOS:27, 33, 38, 39, and 44), a VH CDR2 (SEQ ID NOS:28, 34, 40, 45, and 50), a VH CDR3 (SEQ ID NOS:29, 35, 41, and 46), a VL CDR2 (SEQ ID NOS:31, 37, and 48) and a VL CDR3 (SEQ ID NOS:32, 43, and 49); a VH CDR1 (SEQ ID NOS:27, 33, 38, 39, and 44), a VH CDR2 (SEQ ID NOS:28, 34, 40, 45, and 50), a VL CDR1 (SEQ ID NOS:30, 36, 42, and 47), a VL CDR2 (SEQ ID NOS:31, 37, and 48), and a VL CDR3 (SEQ ID NOS:32, 43, and 49); a VH CDR1 (SEQ ID NOS:27, 33, 38, 39, and 44), a VH CDR3 (SEQ ID NOS:29, 35, 41, and 46), a VL CDR1 (SEQ ID NOS:30, 36, 42, and 47), a VL CDR2 (SEQ ID NOS:31, 37, and 48), and a VL CDR3 (SEQ ID NOS:32, 43, and 49); a VH CDR2 (SEQ ID NOS:28, 34, 40, 45, and 50), a VH CDR3 (SEQ ID NOS:29, 35, 41, and 46), a VL CDR1 (SEQ ID NOS:30, 36, 42, and 47), a VL CDR2 (SEQ ID NOS:31, 37, and 48), and a VL CDR3 (SEQ ID NOS:32, 43, and 49); or any combination thereof of the VH CDRs (SEQ ID NOS: 27, 33, 38, 39, 44, 28, 34, 40, 45, 50, 29, 35, 41, and 46) and VL CDRs (SEQ ID NOS: 30, 36, 42, 47, 31, 37, 48, 32, 43, and 49) listed in Table 2.

In some embodiments, described herein is a multispecific antibody with a first binding domain that binds to B7H3, wherein the first binding domain comprises: (a) a VH region comprising: (1) a VH CDR1 having an amino acid sequence selected from the group consisting of: (i) SEQ ID NO:1, (ii) SEQ ID NO:7, (iii) SEQ ID NO:12, (iv) SEQ ID NO:13, and (v) SEQ ID NO:18; (2) a VH CDR2 having an amino acid sequence selected from the group consisting of: (i) SEQ ID NO:2, (ii) SEQ ID NO:8, (iii) SEQ ID NO:14, (iv) SEQ ID NO:19, and (v) SEQ ID NO:24; and (3) a VH CDR3 having an amino acid sequence selected from the group consisting of: (i) SEQ ID NO:3, (ii) SEQ ID NO:9, (iii) SEQ ID NO:15, and (iv) SEQ ID NO:20; and (b) a VL region comprising: (1) a VL CDR1 having an amino acid sequence selected from the group consisting of: (i) SEQ ID NO:4, (ii) SEQ ID NO:10, (iii) SEQ ID NO:16, and (iv) SEQ ID NO:21; (2) a VL CDR2 having an amino acid sequence selected from the group consisting of: (i) SEQ ID NO:5, (ii) SEQ ID NO:11, and (iii) SEQ ID NO:22; and (3) a VL CDR3 having an amino acid sequence selected from the group consisting of: (i) SEQ ID NO:6, (ii) SEQ ID NO:17, and (iii) SEQ ID NO:23.

In some embodiments, the multispecific antibody comprises a second binding domain that binds to GD2. In some embodiments, the multispecific antibody is a bispecific antibody. In some embodiments, the second binding domain comprises: (a) a VH region comprising: (1) a VH CDR1 having an amino acid sequence selected from the group consisting of: (i) SEQ ID NO:27, (ii) SEQ ID NO:33, (iii) SEQ ID NO:38, (iv) SEQ ID NO:39, and (v) SEQ ID NO:44; (2) a VH CDR2 having an amino acid sequence selected from the group consisting of:(i) SEQ ID NO:28, (ii) SEQ ID NO:34, (iii) SEQ ID NO:40, (iv) SEQ ID NO:45, and (v) SEQ ID NO:50; and (3) a VH CDR3 having an amino acid sequence selected from the group consisting of: (i) SEQ ID NO:29, (ii) SEQ ID NO:35, (iii) SEQ ID NO:41, (iv) SEQ ID NO:46; and (b) a VL region comprising: (1) a VL CDR1 having an amino acid sequence selected from the group consisting of: (i) SEQ ID NO:30, (ii) SEQ ID NO:36, (iii) SEQ ID NO:42, and (iv) SEQ ID NO:47; (2) a VL CDR2 having an amino acid sequence selected from the group consisting of: (i) SEQ ID NO:31, (ii) SEQ ID NO:37, and (iii) SEQ ID NO:48; and (3) a VL CDR3 having an amino acid sequence selected from the group consisting of: (i) SEQ ID NO:32, (ii) SEQ ID NO:43, (iii) SEQ ID NO:49.

In some embodiments, described herein is a multispecific antibody with a first binding domain that binds to B7H3, wherein the first binding domain comprises a VH region comprising: (1) a VH CDR1 having an amino acid sequence selected from the group consisting of: (i) SEQ ID NO:1, (ii) SEQ ID NO:7, (iii) SEQ ID NO:12, (iv) SEQ ID NO:13, and (v) SEQ ID NO:18; (2) a VH CDR2 having an amino acid sequence selected from the group consisting of: (i) SEQ ID NO:2, (ii) SEQ ID NO:8, (iii) SEQ ID NO:14, (iv) SEQ ID NO:19, and (v) SEQ ID NO:24; and (3) a VH CDR3 having an amino acid sequence selected from the group consisting of: (i) SEQ ID NO:3, (ii) SEQ ID NO:9, (iii) SEQ ID NO:15, and (iv) SEQ ID NO:20. In some embodiments, the multispecific antibody comprises a second binding domain that binds to GD2. In some embodiments, the multispecific antibody of is a bispecific antibody. In some embodiments, the second binding domain comprises: (a) a VH region comprising: (1) a VH CDR1 having an amino acid sequence selected from the group consisting of: (i) SEQ ID NO:27, (ii) SEQ ID NO:33, (iii) SEQ ID NO:38, (iv) SEQ ID NO:39, and (v) SEQ ID NO:44; (2) a VH CDR2 having an amino acid sequence selected from the group consisting of: (i) SEQ ID NO:28, (ii) SEQ ID NO:34, (iii) SEQ ID NO:40, (iv) SEQ ID NO:45, and (v) SEQ ID NO:50; and (3) a VH CDR3 having an amino acid sequence selected from the group consisting of: (i) SEQ ID NO:29, (ii) SEQ ID NO:35, (iii) SEQ ID NO:41, and (iv) SEQ ID NO:46.

In some embodiments, described herein is a multispecific antibody with a first binding domain that binds to B7H3, wherein the first binding domain comprises a VL region comprising: (1) a VL CDR1 having an amino acid sequence selected from the group consisting of: (i) SEQ ID NO:4, (ii) SEQ ID NO:10, (iii) SEQ ID NO:16, and (iv) SEQ ID NO:21; (2) a VL CDR2 having an amino acid sequence selected from the group consisting of: (i) SEQ ID NO:5, (ii) SEQ ID NO:11, and (iii) SEQ ID NO:22; (3) a VL CDR3 having an amino acid sequence selected from the group consisting of: (i) SEQ ID NO:6, (ii) SEQ ID NO:17, and (iii) SEQ ID NO:23. In some embodiments, the multispecific antibody comprises a second binding domain that binds to GD2. In some embodiments, the multispecific antibody is a bispecific antibody. In some embodiments, the second binding domain comprises: (a) a VL region comprising: (1) a VL CDR1 having an amino acid sequence selected from the group consisting of: (i) SEQ ID NO:30, (ii) SEQ ID NO:36, (iii) SEQ ID NO:42, and (iv) SEQ ID NO:47; (2) a VL CDR2 having an amino acid sequence selected from the group consisting of: (i) SEQ ID NO:31, (ii) SEQ ID NO:37, and (iii) SEQ ID NO:48; and (3) a VL CDR3 having an amino acid sequence selected from the group consisting of: (i) SEQ ID NO:32, (ii) SEQ ID NO:43, (iii) SEQ ID NO:49.

In some embodiments, described herein is a multispecific antibody with a first binding domain that binds to B7H3, wherein the first binding domain comprises all three heavy chain complementarity determining regions (CDRs) or all three light chain CDRs from: the antibody designated mAb-B7H3 that comprises a VH sequence that is SEQ ID NO:25 and a VL sequence that is SEQ ID NO:26. In some embodiments, the multispecific antibody comprises a second binding domain that binds to GD2. In some embodiments, the multispecific antibody is a bispecific antibody. In some embodiments, the second binding domain comprises all three heavy chain complementarity determining regions (CDRs) or all three light chain CDRs from: the antibody designated mAb-GD2 that comprises a VH sequence that is SEQ ID NO:51 and a VL sequence that is SEQ ID NO:52.

In some embodiments, described herein is a multispecific antibody with a first binding domain that binds to B7H3, wherein the first binding domain comprises all three heavy chain CDRs and/or all three light chain CDRs from the antibody designated mAb-B7H3. In some embodiments, the multispecific antibody comprises a second binding domain that binds to GD2. In some embodiments, the multispecific antibody of is a bispecific antibody. In some embodiments, the second binding domain comprises all three heavy chain CDRs and all three light chain CDRs from the antibody designated mAb-GD2.

In some embodiments, described herein is a multispecific antibody with a first binding domain that binds to B7H3, wherein the first binding domain comprises: (a) a VH region comprising a VH CDR1, a VH CDR2, and a VH CDR3 amino acid sequence depicted in Table 1; or (b) a VL region comprising a VL CDR1, a VL CDR2, and a VL CDR3 amino acid sequence depicted in Table 1. In some embodiments, the first binding domain comprises: (a) a VH region comprising a VH CDR1, a VH CDR2, and a VH CDR3 amino acid sequence depicted in Table 1; and (b) a VL region comprising a VL CDR1, a VL CDR2, and a VL CDR3 amino acid sequence depicted in Table 1. In some embodiments, the multispecific antibody comprises a second binding domain that binds to GD2. In some embodiments, the multispecific antibody is a bispecific antibody. In some embodiments, the second binding domain comprises: (a) a VH region comprising a VH CDR1, a VH CDR2, and a VH CDR3 amino acid sequence depicted in Table 2; and/or (b) a VL region comprising a VL CDR1, a VL CDR2, and a VL CDR3 amino acid sequence depicted in Table 2. In some embodiments, the first binding domain comprises a VH region comprising a VH CDR1, a VH CDR2, and a VH CDR3 amino acid sequence depicted in Table 1. In some embodiments, the multispecific antibody comprises a second binding domain that binds to GD2. In some embodiments, the multispecific antibody is a bispecific antibody. In some embodiments, the second binding domain comprises a VH region comprising a VH CDR1, a VH CDR2, and a VH CDR3 amino acid sequence depicted in Table 2. In some embodiments, the first binding domain comprises a VL region comprising a VL CDR1, a VL CDR2, and a VL CDR3 amino acid sequence depicted in Table 1. In some embodiments, the multispecific antibody comprises a second binding domain that binds to GD2. In some embodiments, the multispecific antibody is a bispecific antibody. In some embodiments, the second binding domain comprises a VL region comprising a VL CDR1, a VL CDR2, and a VL CDR3 amino acid sequence depicted in Table 2.

In some embodiments, described herein is a multispecific antibody with a first binding domain that binds to B7H3, wherein the first binding domain comprises: (a) a VH region comprising: (1) a VH CDR1 having an amino acid sequence selected from the group consisting of SEQ ID NO:1, 7, 12, 13, and 18; (2) a VH CDR2 having an amino acid sequence selected from the group consisting of SEQ ID NO:2, 8, 14, 19 and 24; and (3) a VH CDR3 having an amino acid sequence selected from the group consisting of SEQ ID NO:3, 9, 15 and 20; and (b) a VL region comprising: (1) a VL CDR1 having an amino acid sequence selected from the group consisting of SEQ ID NO:4, 10, 16 and 21; (2) a VL CDR2 having an amino acid sequence selected from the group consisting of SEQ ID NO:5, 11, and 22; and (3) a VL CDR3 having an amino acid sequence selected from the group consisting of SEQ ID NO:6, 17, and 23. In some embodiments, the multispecific antibody comprises a second binding domain that binds to GD2. In some embodiments, the first binding domain comprises: (a) a VH region comprising: (1) a VH CDR1 having the amino acid sequence of SEQ ID NO:1; (2) a VH CDR2 having the amino acid sequence of SEQ ID NO:2; and (3) a VH CDR3 having the amino acid sequence of SEQ ID NO:3; and (b) a VL region comprising: (1) a VL CDR1 having the amino acid sequence of SEQ ID NO:4; (2) a VL CDR2 having the amino acid sequence of SEQ ID NO:5; and (3) a VL CDR3 having the amino acid sequence of SEQ ID NO:6. In some embodiments, the second binding domain comprises: (a) a VH region comprising: (1) a VH CDR1 having the amino acid sequence of SEQ ID NO:27; (2) a VH CDR2 having the amino acid sequence of SEQ ID NO:28; and (3) a VH CDR3 having the amino acid sequence of SEQ ID NO:29; and (b) a VL region comprising: (1) a VL CDR1 having the amino acid sequence of SEQ ID NO:30; (2) a VL CDR2 having the amino acid sequence of SEQ ID NO:31; and (3) a VL CDR3 having the amino acid sequence of SEQ ID NO:32. In some embodiments, wherein the first binding domain comprises: (a) a VH region comprising: (1) a VH CDR1 having the amino acid sequence of SEQ ID NO:7; (2) a VH CDR2 having the amino acid sequence of SEQ ID NO:8; and (3) a VH CDR3 having the amino acid sequence of SEQ ID NO:9; and (b) a VL region comprising: (1) a VL CDR1 having the amino acid sequence of SEQ ID NO:10; (2) a VL CDR2 having the amino acid sequence of SEQ ID NO:11; and (3) a VL CDR3 having the amino acid sequence of SEQ ID NO:6. In some embodiments, the second binding domain comprises: (a) a VH region comprising: (1) a VH CDR1 having the amino acid sequence of SEQ ID NO:33; (2) a VH CDR2 having the amino acid sequence of SEQ ID NO:34; and (3) a VH CDR3 having the amino acid sequence of SEQ ID NO:35; and (b) a VL region comprising: (1) a VL CDR1 having the amino acid sequence of SEQ ID NO:36; (2) a VL CDR2 having the amino acid sequence of SEQ ID NO:37; and (3) a VL CDR3 having the amino acid sequence of SEQ ID NO:38. In some embodiments, the first binding domain comprises: (a) a VH region comprising: (1) a VH CDR1 having the amino acid sequence of SEQ ID NO:12; (2) a VH CDR2 having the amino acid sequence of SEQ ID NO:2; and (3) a VH CDR3 having the amino acid sequence of SEQ ID NO:3; and (b) a VL region comprising: (1) a VL CDR1 having the amino acid sequence of SEQ ID NO:4; (2) a VL CDR2 having the amino acid sequence of SEQ ID NO:5; and (3) a VL CDR3 having the amino acid sequence of SEQ ID NO:6. In some embodiments, the second binding domain comprises: (a) a VH region comprising: (1) a VH CDR1 having the amino acid sequence of SEQ ID NO:38; (2) a VH CDR2 having the amino acid sequence of SEQ ID NO:28; and (3) a VH CDR3 having the amino acid sequence of SEQ ID NO:29; and (b) a VL region comprising: (1) a VL CDR1 having the amino acid sequence of SEQ ID NO:30; (2) a VL CDR2 having the amino acid sequence of SEQ ID NO:31; and (3) a VL CDR3 having the amino acid sequence of SEQ ID NO:32. In some embodiments, first binding domain comprises: (a) a VH region comprising: (1) a VH CDR1 having the amino acid sequence of SEQ ID NO:13; (2) a VH CDR2 having the amino acid sequence of SEQ ID NO:14; and (3) a VH CDR3 having the amino acid sequence of SEQ ID NO:15; and (b) a VL region comprising: (1) a VL CDR1 having the amino acid sequence of SEQ ID NO:16; (2) a VL CDR2 having the amino acid sequence of SEQ ID NO:11; and (3) a VL CDR3 having the amino acid sequence of SEQ ID NO:17. In some embodiments, the second binding domain comprises: (a) a VH region comprising: (1) a VH CDR1 having the amino acid sequence of SEQ ID NO:39; (2) a VH CDR2 having the amino acid sequence of SEQ ID NO:40; and (3) a VH CDR3 having the amino acid sequence of SEQ ID NO:41; and (b) a VL region comprising: (1) a VL CDR1 having the amino acid sequence of SEQ ID NO:42; (2) a VL CDR2 having the amino acid sequence of SEQ ID NO:37; and (3) a VL CDR3 having the amino acid sequence of SEQ ID NO:43. In some embodiments, first binding domain comprises: (a) a VH region comprising: (1) a VH CDR1 having the amino acid sequence of SEQ ID NO:18; (2) a VH CDR2 having the amino acid sequence of SEQ ID NO:19; and (3) a VH CDR3 having the amino acid sequence of SEQ ID NO:20; and (b) a VL region comprising: (1) a VL CDR1 having the amino acid sequence of SEQ ID NO:21; (2) a VL CDR2 having the amino acid sequence of SEQ ID NO:22; and (3) a VL CDR3 having the amino acid sequence of SEQ ID NO:23. In some embodiments, the second binding domain comprises: (a) a VH region comprising: (1) a VH CDR1 having the amino acid sequence of SEQ ID NO:44; (2) a VH CDR2 having the amino acid sequence of SEQ ID NO:45; and (3) a VH CDR3 having the amino acid sequence of SEQ ID NO:46; and (b) a VL region comprising: (1) a VL CDR1 having the amino acid sequence of SEQ ID NO:47; (2) a VL CDR2 having the amino acid sequence of SEQ ID NO:48; and (3) a VL CDR3 having the amino acid sequence of SEQ ID NO:49. In some embodiments, the first binding domain comprises: (a) a VH region comprising: (1) a VH CDR1 having the amino acid sequence of SEQ ID NO:1; (2) a VH CDR2 having the amino acid sequence of SEQ ID NO:24; and (3) a VH CDR3 having the amino acid sequence of SEQ ID NO:3; and (b) a VL region comprising: (1) a VL CDR1 having the amino acid sequence of SEQ ID NO:4; (2) a VL CDR2 having the amino acid sequence of SEQ ID NO:5; and (3) a VL CDR3 having the amino acid sequence of SEQ ID NO:6. In some embodiments, the second binding domain comprises: (a) a VH region comprising: (1) a VH CDR1 having the amino acid sequence of SEQ ID NO:27; (2) a VH CDR2 having the amino acid sequence of SEQ ID NO:50; and (3) a VH CDR3 having the amino acid sequence of SEQ ID NO:29; and (b) a VL region comprising: (1) a VL CDR1 having the amino acid sequence of SEQ ID NO:30; (2) a VL CDR2 having the amino acid sequence of SEQ ID NO:31; and (3) a VL CDR3 having the amino acid sequence of SEQ ID NO:32. In some embodiments, a multispecific antibody described above is a bispecific antibody.

In some embodiments, described herein is a multispecific antibody comprising a VH region and/or VL region described herein, which further comprises human framework sequences. In some embodiment, the VH region and/or VL region further comprises a framework 1 (FR1), a framework 2 (FR2), a framework 3 (FR3) and/or a framework 4 (FR4) sequence.

In some embodiments, the multispecific antibody described herein is a monoclonal antibody. In some embodiments, the monoclonal antibody is a humanized, human or chimeric antibody. In some embodiments, the multispecific antibody described herein is a recombinant antibody, which is optionally a humanized, human or chimeric antibody

In certain aspects, the CDRs of a multispecific binding agent (e.g., an antibody, such as a bispecific antibody), including a multispecific binding agent that binds to B7H3, including human B7H3, and GD2, including human GD2, can be determined according to the Kabat system (Kabat et al. (1971) Ann. NY Acad. Sci. 190:382-391 and, Kabat et al. (1991) Sequences of Proteins of Immunological Interest, Fifth Edition, U.S. Department of Health and Human Services, NIH Publication No. 91-3242).

In certain aspects, the CDRs of a multispecific binding agent (e.g., an antibody, such as a bispecific antibody), including a multispecific binding agent that binds to B7H3, including human B7H3, and GD2, including human GD2, can be determined according to the Chothia system, which will be referred to herein as the “Chothia CDRs” (see, e.g., Chothia and Lesk, 1987, J. Mol. Biol., 196:901-917; Al-Lazikani et al., 1997, J. Mol. Biol., 273:927-948; Chothia et al., 1992, J. Mol. Biol., 227:799-817; Tramontano A et al., 1990, J. Mol. Biol. 215(1):175-82; and U.S. Pat. No. 7,709,226).

In certain aspects, the CDRs of a multispecific binding agent (e.g., an antibody, such as a bispecific antibody), including a multispecific binding agent that binds to B7H3, including human B7H3, and GD2, including human GD2, can be determined according to the ImMunoGeneTics (IMGT) system, for example, as described in Lefranc, M.-P., 1999, The Immunologist, 7:132-136 and Lefranc, M.-P. et al., 1999, Nucleic Acids Res., 27:209-212 (“IMGT CDRs”).

In certain aspects, the CDRs of a multispecific binding agent (e.g., an antibody, such as a bispecific antibody), including a multispecific binding agent that binds to B7H3, including human B7H3, and GD2, including human GD2, can be determined according to the AbM system, which will be referred to herein as the “AbM CDRs,” for example as described in MacCallum et al., 1996, J. Mol. Biol., 262:732-745. See also, e.g., Martin, A., “Protein Sequence and Structure Analysis of Antibody Variable Domains,” in Antibody Engineering, Kontermann and Dubel, eds., Chapter 31, pp. 422-439, Springer-Verlag, Berlin (2001).

In certain aspects, the CDRs of a multispecific binding agent (e.g., an antibody, such as a bispecific antibody), including a multispecific binding agent that binds to B7H3, including human B7H3, and GD2, including human GD2, can be determined according to the Contact system, which will be referred to herein as the “Contact CDRs” (see, e.g., MacCallum R M et al., 1996, J Mol Biol 5: 732-745). The Contact CDRs are based on an analysis of the available complex crystal structures.

In some embodiments, the position of one or more CDRs along the VH (e.g., CDR1, CDR2, or CDR3) and/or VL (e.g., CDR1, CDR2, or CDR3) region of a first binding domain of a multispecific binding agent (e.g., an antibody, such as a bispecific antibody), including a multispecific binding agent that binds to B7H3, including human B7H3, and GD2, including human GD2, described herein may vary by one, two, three, four, five, or six amino acid positions so long as binding to B7H3 (e.g., human B7H3) is maintained (e.g., substantially maintained, for example, at least 50%, at least 60%, at least 70%, at least 80%, at least 90%, at least 95%). For example, in some embodiments, the position defining a CDR of Table 1 may vary by shifting the N-terminal and/or C-terminal boundary of the CDR by one, two, three, four, five, or six amino acids, relative to the current CDR position, so long as binding to B7H3 (e.g., human B7H3) is maintained (e.g., substantially maintained, for example, at least 50%, at least 60%, at least 70%, at least 80%, at least 90%, at least 95%). In other embodiments, the length of one or more CDRs along the VH (e.g., CDR1, CDR2, or CDR3) and/or VL (e.g., CDR1, CDR2, or CDR3) region of a first binding domain of a multispecific binding agent (e.g., an antibody, such as a bispecific antibody), including a multispecific binding agent that binds to B7H3, including human B7H3, and GD2, including human GD2, described herein may vary (e.g., be shorter or longer) by one, two, three, four, five, or more amino acids, so long as binding to B7H3 (e.g., human B7H3) is maintained (e.g., substantially maintained, for example, at least 50%, at least 60%, at least 70%, at least 80%, at least 90%, at least 95%). For example, in some embodiments, a VH and/or VL CDR1, CDR2, and/or CDR3 described herein may be one, two, three, four, five or more amino acids shorter than one or more of the CDRs described by SEQ ID NOS:1-24, so long as binding to B7H3 (e.g., human B7H3) is maintained (e.g., substantially maintained, for example, at least 50%, at least 60%, at least 70%, at least 80%, at least 90%, at least 95%). In other embodiments, a VH and/or VL CDR1, CDR2, and/or CDR3 described herein may be one, two, three, four, five or more amino acids longer than one or more of the CDRs described by SEQ ID NOS:1-24, so long as binding to B7H3 (e.g., human B7H3) is maintained (e.g., substantially maintained, for example, at least 50%, at least 60%, at least 70%, at least 80%, at least 90%, at least 95%). In other embodiments, the amino terminus of a VH and/or VL CDR1, CDR2, and/or CDR3 of a first binding domain described herein may be extended by one, two, three, four, five or more amino acids compared to one or more of the CDRs described by SEQ ID NOS:1-24, so long as binding to B7H3 (e.g., human B7H3) is maintained (e.g., substantially maintained, for example, at least 50%, at least 60%, at least 70%, at least 80%, at least 90%, at least 95%). In other embodiments, the carboxy terminus of a VH and/or VL CDR1, CDR2, and/or CDR3 of a first binding domain described herein may be extended by one, two, three, four, five or more amino acids compared to one or more of the CDRs described by SEQ ID NOS:1-24, so long as binding to B7H3 (e.g., human B7H3) is maintained (e.g., substantially maintained, for example, at least 50%, at least 60%, at least 70%, at least 80%, at least 90%, at least 95%). In other embodiments, the amino terminus of a VH and/or VL CDR1, CDR2, and/or CDR3 of a first binding domain described herein may be shortened by one, two, three, four, five or more amino acids compared to one or more of the CDRs described by SEQ ID NOS:1-24, so long as binding to B7H3 (e.g., human B7H3) is maintained (e.g., substantially maintained, for example, at least 50%, at least 60%, at least 70%, at least 80%, at least 90%, at least 95%). In some embodiments, the carboxy terminus of a VH and/or VL CDR1, CDR2, and/or CDR3 of a first binding domain described herein may be shortened by one, two, three, four, five or more amino acids compared to one or more of the CDRs described by SEQ ID NOS:1-24, so long as binding to B7H3 (e.g., human B7H3) is maintained (e.g., substantially maintained, for example, at least 50%, at least 60%, at least 70%, at least 80%, at least 90%, at least 95%). Any method known in the art can be used to ascertain whether binding to B7H3 (e.g., human B7H3) is maintained, for example, the binding assays and conditions described in the “Examples” section described herein.

In some embodiments, the position of one or more CDRs along the VH (e.g., CDR1, CDR2, or CDR3) and/or VL (e.g., CDR1, CDR2, or CDR3) region of a second binding domain of a multispecific binding agent (e.g., an antibody, such as a bispecific antibody), including a multispecific binding agent that binds to B7H3, including human B7H3, and GD2, including human GD2, described herein may vary by one, two, three, four, five, or six amino acid positions so long as binding to GD2 (e.g., human GD2) is maintained (e.g., substantially maintained, for example, at least 50%, at least 60%, at least 70%, at least 80%, at least 90%, at least 95%). For example, in some embodiments, the position defining a CDR of Table 2 may vary by shifting the N-terminal and/or C-terminal boundary of the CDR by one, two, three, four, five, or six amino acids, relative to the current CDR position, so long as binding to GD2 (e.g., human GD2) is maintained (e.g., substantially maintained, for example, at least 50%, at least 60%, at least 70%, at least 80%, at least 90%, at least 95%). In other embodiments, the length of one or more CDRs along the VH (e.g., CDR1, CDR2, or CDR3) and/or VL (e.g., CDR1, CDR2, or CDR3) region of a second binding domain of a multispecific binding agent (e.g., an antibody, such as a bispecific antibody), including a multispecific binding agent that binds to B7H3, including human B7H3, and GD2, including human GD2, described herein may vary (e.g., be shorter or longer) by one, two, three, four, five, or more amino acids, so long as binding to GD2 (e.g., human GD2) is maintained (e.g., substantially maintained, for example, at least 50%, at least 60%, at least 70%, at least 80%, at least 90%, at least 95%). For example, in some embodiments, a VH and/or VL CDR1, CDR2, and/or CDR3 described herein may be one, two, three, four, five or more amino acids shorter than one or more of the CDRs described by SEQ ID NOS:27-50, so long as binding to GD2 (e.g., human GD2) is maintained (e.g., substantially maintained, for example, at least 50%, at least 60%, at least 70%, at least 80%, at least 90%, at least 95%). In other embodiments, a VH and/or VL CDR1, CDR2, and/or CDR3 described herein may be one, two, three, four, five or more amino acids longer than one or more of the CDRs described by SEQ ID NOS:27-50, so long as binding to GD2 (e.g., human GD2) is maintained (e.g., substantially maintained, for example, at least 50%, at least 60%, at least 70%, at least 80%, at least 90%, at least 95%). In other embodiments, the amino terminus of a VH and/or VL CDR1, CDR2, and/or CDR3 of a second binding domain described herein may be extended by one, two, three, four, five or more amino acids compared to one or more of the CDRs described by SEQ ID NOS:27-50, so long as binding to GD2 (e.g., human GD2) is maintained (e.g., substantially maintained, for example, at least 50%, at least 60%, at least 70%, at least 80%, at least 90%, at least 95%). In other embodiments, the carboxy terminus of a VH and/or VL CDR1, CDR2, and/or CDR3 of a second binding domain described herein may be extended by one, two, three, four, five or more amino acids compared to one or more of the CDRs described by SEQ ID NOS:27-50, so long as binding to GD2 (e.g., human GD2) is maintained (e.g., substantially maintained, for example, at least 50%, at least 60%, at least 70%, at least 80%, at least 90%, at least 95%). In other embodiments, the amino terminus of a VH and/or VL CDR1, CDR2, and/or CDR3 of a second binding domain described herein may be shortened by one, two, three, four, five or more amino acids compared to one or more of the CDRs described by SEQ ID NOS:27-50, so long as binding to GD2 (e.g., human GD2) is maintained (e.g., substantially maintained, for example, at least 50%, at least 60%, at least 70%, at least 80%, at least 90%, at least 95%). In some embodiments, the carboxy terminus of a VH and/or VL CDR1, CDR2, and/or CDR3 of a second binding domain described herein may be shortened by one, two, three, four, five or more amino acids compared to one or more of the CDRs described by SEQ ID NOS:27-50, so long as binding to GD2 (e.g., human GD2) is maintained (e.g., substantially maintained, for example, at least 50%, at least 60%, at least 70%, at least 80%, at least 90%, at least 95%). Any method known in the art can be used to ascertain whether binding to GD2 (e.g., human GD2) is maintained, for example, the binding assays and conditions described in the “Examples” section described herein.

In other embodiments, the multispecific binding agents (e.g., antibodies, such as bispecific antibodies), including multispecific binding agents that bind to B7H3, including human B7H3, and GD2, including human GD2, presented herein that bind to B7H3, comprise conservative sequence modifications. With respect to polypeptides that are multispecific binding agents (e.g., antibodies, such as bispecific antibodies), such as multispecific binding agents that bind to B7H3, including human B7H3, and GD2, including human GD2, conservative sequence modifications include conservative amino acid substitutions that include ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, in some embodiments, a predicted nonessential amino acid residue in a B7H3 or GD2 is replaced with another amino acid residue from the same side chain family. Methods of identifying nucleotide and amino acid conservative substitutions which do not eliminate antigen binding are well-known in the art (see, e.g., Brummell et al., Biochem. 32:1180-1187 (1993); Kobayashi et al. Protein Eng. 12(10):879-884 (1999); and Burks et al. Proc. Natl. Acad. Sci. USA 94:412-417 (1997)). In some embodiments, the conservative sequence modifications described herein modify the amino acid sequences of the multispecific binding agents (e.g., antibodies, such as bispecific antibodies), including multispecific binding agents that bind to B7H3, including human B7H3, and GD2, including human GD2, by 50%, or 55%, or 60%, or 65%, or 70%, or 75%, or 80%, or 85%, or 90%, or 95%, or 98%, or 99%. In some embodiments, the nucleotide and amino acid sequence modifications refer to at most 1, 2, 3, 4, 5, or 6 amino acid substitutions to the CDRs described in Table 1 or Table 2. Thus, for example, each such CDR may contain up to 5 conservative amino acid substitutions, for example up to (not more than) 4 conservative amino acid substitutions, for example up to (not more than) 3 conservative amino acid substitutions, for example up to (not more than) 2 conservative amino acid substitutions, or no more than 1 conservative amino acid substitution.

The present disclosure provides multispecific binding agents (e.g., antibodies, such as bispecific antibodies), including multispecific binding agents that bind to B7H3, including human B7H3, and GD2, including human GD2 with a masking moiety and/or cleavable moiety in which one or more of the B7H3 and/or other target binding domains of the multispecific binding agent (e.g., an antibody, such as a bispecific antibody) are masked (e.g., via a masking moiety) and/or activatable (e.g., via a cleavable moiety). Technologies for masking of a multispecific binding agent (e.g., an antibody, such as a bispecific antibody) are well known in the art, including SAFE body masking technology (see, e.g., US Patent Application Publication No. 2019/0241886) and Probody masking technology (see, e.g., US Patent Application Publication No. 2015/0079088). Such technologies can be used to generate a multispecific binding agent (e.g., an antibody, such as a bispecific antibody) that is masked and/or activatable. Such masked and/or activatable multispecific binding agents (e.g., antibodies, such as bispecific antibodies), including multispecific binding agents that bind to B7H3, including human B7H3, and GD2, including human GD2 are useful for the preparation of conjugates, including immunoconjugates, antibody-drug conjugates (ADCs), masked ADCs and activatable antibody-drug conjugates (AADCs), comprising any one of the multispecific binding agents (e.g., antibodies, such as bispecific antibodies), including multispecific binding agents that bind to B7H3, including human B7H3, and GD2, including human GD2, such as human B7H3 binding agents, of the present disclosure, including those directly or indirectly linked to another agent such as a drug. For example, multispecific binding agents (e.g., antibodies, such as bispecific antibodies), including multispecific binding agents that bind to B7H3, including human B7H3, and GD2, including human GD2, such as human B7H3 binding agents, of the present disclosure may be covalently bound by a synthetic linker to one or more agents such as drugs.

If desired, a multispecific binding agent (e.g., an antibody, such as a bispecific antibody) is linked or conjugated (directly or indirectly) to a moiety with effector function, such as cytotoxic activity (e.g., a chemotherapeutic moiety or a radioisotope) or immune recruitment activity. Moieties that are linked or conjugated (directly or indirectly) include drugs that are cytotoxic (e.g., toxins such as aurostatins) or non-cytotoxic (e.g., signal transduction modulators such as kinases or masking moieties that mask one or more binding domains of a multispecific binding agent (e.g., an antibody, such as a bispecific antibody), or cleavable moieties that allow for activating a multispecific binding agent by cleaving of a cleavable moiety to unmask one or more binding domains of a multispecific binding agent (e.g., an antibody, such as a bispecific antibody) in the tumor microenvironment) in the form of masked conjugates. Moieties that promote immune recruitment can include other antigen-binding agents, such as viral proteins that bind selectively to cells of the innate immune system. Alternatively or in addition, a multispecific binding agent (e.g., an antibody, such as a bispecific antibody) is optionally linked or conjugated (directly or indirectly) to a moiety that facilitates isolation from a mixture (e.g., a tag) or a moiety with reporter activity (e.g., a detection label or reporter protein). It will be appreciated that the features of a multispecific binding agent (e.g., an antibody, such as a bispecific antibody) described herein extend also to a polypeptide comprising a binding agent fragment.

In some embodiments, multispecific binding agents (e.g., antibodies, such as bispecific antibodies), including multispecific binding agents that bind to B7H3, including human B7H3, and GD2, including human GD2 described herein may be linked or conjugated (directly or indirectly) to a polypeptide, which can result in the generation of an activatable antibody. In some embodiments, a multispecific binding agent (e.g., an antibody, such as a bispecific antibody) is linked or conjugated (directly or indirectly) to an agent. In some embodiments, the agent is a drug, resulting in an ADC or an AADC when the antibody of the ADC comprises a masking moiety and a cleavable moiety.

In some embodiments, multispecific binding agents (e.g., antibodies, such as bispecific antibodies), including multispecific binding agents that bind to B7H3, including human B7H3, and GD2, including human GD2 described herein are conjugated or recombinantly linked (directly or indirectly) to a therapeutic agent (e.g., a cytotoxic agent) or to a diagnostic or detectable agent. The conjugated or recombinantly linked antibodies, including masked or activatable conjugates, can be useful, for example, for treating or preventing a disease or disorder such as cancer. The conjugated or recombinantly linked multispecific binding agents (e.g., antibodies, such as bispecific antibodies), including multispecific binding agents that bind to B7H3, including human B7H3, and GD2, including human GD2, including masked or activatable conjugates, can be useful, for example, for monitoring or prognosing the onset, development, progression, and/or severity of a cancer.

Such diagnosis and detection can be accomplished, for example, by coupling the multispecific binding agent (e.g., an antibody, such as a bispecific antibody) to detectable substances including, for example: enzymes, including, but not limited to, horseradish peroxidase, alkaline phosphatase, beta-galactosidase, or acetylcholinesterase; prosthetic groups, including, but not limited to, streptavidin/biotin or avidin/biotin; fluorescent materials, including, but not limited to, umbelliferone, fluorescein, fluorescein isothiocynate, rhodamine, dichlorotriazinylamine fluorescein, dansyl chloride, or phycoerythrin; luminescent materials, including, but not limited to, luminol; bioluminescent materials, including, but not limited to, luciferase, luciferin, or aequorin; chemiluminescent material, including, but not limited to, an acridinium based compound or a HALOTAG; radioactive materials, including, but not limited to, iodine (131I, 125I, 123I, and 121I), carbon (14C), sulfur (35S), tritium (3H), indium (115In, 113In, 112In, and 111In), technetium (99Tc), thallium (201Ti), gallium (68Ga and 67Ga), palladium (103Pd), molybdenum (99Mo), xenon (133Xe), fluorine (18F), 153Sm, 177Lu, 159Gd, 149Pm, 140La, 175Yb, 166Ho, 90Y, 47Sc, 186Re, 188Re, 142Pr, 105Rh, 97Ru, 68Ge, 57Co, 65Zn, 85Sr, 32P, 153Gd, 169Yb, 51Cr, 54Mn, 75Se, 113Sn, or 117Sn; positron emitting metals using various positron emission tomographies; and non-radioactive paramagnetic metal ions.

Also described herein are multispecific binding agents (e.g., antibodies, such as bispecific antibodies) that are recombinantly linked or conjugated (covalent or non-covalent conjugations, directly or indirectly) to a heterologous protein or polypeptide (or fragment thereof, for example, to a polypeptide (e.g., of about 10, about 20, about 30, about 40, about 50, about 60, about 70, about 80, about 90, or about 100 amino acids) to generate fusion proteins, as well as uses thereof. In particular, described herein are fusion proteins comprising an antigen-binding fragment of a multispecific binding agent (e.g., an antibody, such as a bispecific antibody), including a multispecific binding agent that binds to B7H3, including human B7H3, and GD2, including human GD2, described herein (e.g., comprising CDR1, CDR2, and/or CDR3 of VH and/or VL) and a heterologous protein, polypeptide, or peptide. In some embodiments, the heterologous protein, polypeptide, or peptide that a multispecific binding agent (e.g., an antibody, such as a bispecific antibody) is linked to is useful for targeting the multispecific binding agent to a particular cell (e.g., a B7H3 expressing cell and/or GD2 expressing cell, including a tumor cell).

Moreover, multispecific binding agents (e.g., antibodies, such as bispecific antibodies), including multispecific binding agents that bind to B7H3, including human B7H3, and GD2, including human GD2, described herein can be linked (directly or indirectly) to marker or “tag” sequences, such as a peptide, to facilitate purification. In some embodiments, the marker or tag amino acid sequence is a hexa-histidine peptide, such as the tag provided in a pQE vector (see, e.g., QIAGEN, Inc.), among others, many of which are commercially available. For example, as described in Gentz et al., 1989, Proc. Natl. Acad. Sci. USA 86:821-24, hexa-histidine provides for convenient purification of a fusion protein. Other peptide tags useful for purification include, but are not limited to, the hemagglutinin (“HA”) tag, which corresponds to an epitope derived from the influenza hemagglutinin protein (Wilson et al., 1984, Cell 37:767-78), and the “FLAG” tag.

Methods for linking or conjugating (directly or indirectly) moieties (including polypeptides) to antibodies are well known in the art, any one of which can be used to make an antibody-drug conjugate or fusion protein described herein.

In some embodiments, a multispecific binding agent (e.g., an antibody, such as a bispecific antibody) described herein is a fusion protein. The term “fusion protein” as used herein refers to a polypeptide that comprises an amino acid sequence of a binding agent (e.g., an antibody, such as a bispecific antibody) and an amino acid sequence of a heterologous polypeptide or protein (e.g., a polypeptide or protein not normally a part of the antibody (e.g., a non-B7H3 binding antibody or a non-GD2 binding antibody)). In certain embodiments, the fusion protein retains the biological activity of a multispecific binding agent. In certain embodiments, the fusion protein comprises a first binding domain that comprises B7H3 antibody VH region, VL region, VH CDR (one, two or three VH CDRs), and/or VL CDR (one, two or three VL CDRs), wherein the fusion protein binds to a B7H3 epitope, a B7H3 fragment and/or a B7H3 polypeptide. In certain embodiments, the fusion protein comprises a second binding domain that comprises GD2 antibody VH region, VL region, VH CDR (one, two or three VH CDRs), and/or VL CDR (one, two or three VL CDRs), wherein the fusion protein binds to a GD2 epitope, a GD2 fragment and/or a GD2 polypeptide.

Fusion proteins may be generated, for example, through the techniques of gene-shuffling, motif-shuffling, exon-shuffling, and/or codon-shuffling (collectively referred to as “DNA shuffling”). DNA shuffling may be employed to alter the activities of the multispecific binding agents (e.g., antibodies, such as bispecific antibodies), including multispecific binding agents that bind to B7H3, including human B7H3, and GD2, including human GD2, as described herein, including, for example, multispecific binding agents with higher affinities and lower dissociation rates (see, e.g., U.S. Pat. Nos. 5,605,793; 5,811,238; 5,830,721; 5,834,252; and U.S. Pat. No. 5,837,458; Patten et al., 1997, Curr. Opinion Biotechnol. 8:724-33; Harayama, 1998, Trends Biotechnol. 16(2):76-82; Hansson et al., 1999, J. Mol. Biol. 287:265-76; and Lorenzo and Blasco, 1998, Biotechniques 24(2):308-13). In some embodiments, multispecific binding agents, including multispecific binding agents that bind to B7H3, including human B7H3, and GD2, including human GD2, may be altered by being subjected to random mutagenesis by error-prone PCR, random nucleotide insertion, or other methods prior to recombination. A polynucleotide encoding a multispecific binding agent described herein may be recombined with one or more components, motifs, sections, parts, domains, fragments, etc. of one or more heterologous molecules.

Multispecific binding agents (e.g., antibodies, such as bispecific antibodies), including multispecific binding agents that bind to B7H3, including human B7H3, and GD2, including human GD2, described herein may also be attached to solid supports, which are useful for immunoassays or purification of the target antigen. Such solid supports include, but are not limited to, glass, cellulose, polyacrylamide, nylon, polystyrene, polyvinyl chloride, or polypropylene.

Multispecific binding agents (e.g., antibodies, such as bispecific antibodies), including multispecific binding agents that bind to B7H3, including human B7H3, and GD2, including human GD2, described herein can also be linked or conjugated (directly or indirectly) to a second antibody to form an antibody heteroconjugate.

The linker may be a “cleavable moiety” facilitating release of the linked or conjugated agent in a cell, but non-cleavable linkers are also contemplated herein. Linkers for use in conjugates (e.g., antibody-drug conjugates) of the present disclosure include, without limitation, acid labile linkers (e.g., hydrazone linkers), disulfide-containing linkers, peptidase-sensitive linkers (e.g., peptide linkers comprising amino acids, for example, valine and/or citrulline such as citrulline-valine or phenylalanine-lysine), photolabile linkers, dimethyl linkers, thioether linkers, or hydrophilic linkers designed to evade multidrug transporter-mediated resistance.

Conjugates of an antibody and agent, including wherein the agent is a drug for the preparation of an ADC or an AADC, may be made using a variety of bifunctional protein coupling agents such as BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, sulfo-SMPB, and SVSB (succinimidyl-(4-vinylsulfone)benzoate). The present disclosure further contemplates that conjugates of antibodies and agents, including wherein the agent is a drug for the preparation of an ADC or an AADC, may be prepared using any suitable methods as disclosed in the art (see, e.g., Bioconjugate Techniques (Hermanson ed., 2d ed. 2008)).

Conventional conjugation strategies for antibodies and agents, including wherein the agent is a drug for the preparation of an ADC or an AADC, have been based on random conjugation chemistries involving the ε-amino group of Lys residues or the thiol group of Cys residues, which results in heterogeneous conjugates. Recently developed techniques allow site-specific conjugation to antibodies, resulting in homogeneous loading and avoiding conjugate subpopulations with altered antigen-binding or pharmacokinetics. These include engineering of “thiomabs” comprising cysteine substitutions at positions on the heavy and light chains that provide reactive thiol groups and do not disrupt immunoglobulin folding and assembly or alter antigen binding (see, e.g., Junutula et al., 2008, J. Immunol. Meth. 332: 41-52; and Junutula et al., 2008, Nature Biotechnol. 26:925-32). In another method, selenocysteine is cotranslationally inserted into an antibody sequence by recoding the stop codon UGA from termination to selenocysteine insertion, allowing site specific covalent conjugation at the nucleophilic selenol group of selenocysteine in the presence of the other natural amino acids (see, e.g., Hofer et al., 2008, Proc. Natl. Acad. Sci. USA 105:12451-56; and Hofer et al., 2009, Biochemistry 48(50):12047-57).

In some embodiments, a multispecific binding agent (e.g., an antibody, such as a bispecific antibody), including a multispecific binding agent that binds to B7H3, including human B7H3, and GD2, including human GD2, described herein is conjugated to a cytotoxic agent. In some embodiments, a multispecific binding agent (e.g., an antibody, such as a bispecific antibody), including a multispecific binding agent that binds to B7H3, including human B7H3, and GD2, including human GD2, disclosed herein can be optionally conjugated with one or more cytotoxic agent(s) disclosed herein or known in the art in order to generate an ADC or an AADC. In some embodiments, the cytotoxic agent is a chemotherapeutic agent including, but not limited to, methotrexate, adriamycin, doxorubicin, melphalan, mitomycin C, chlorambucil, daunorubicin or other intercalating agents. In some embodiments, the cytotoxic agent is an enzymatically active toxin of bacterial, fungal, plant, or animal origin, or fragments thereof, including, but not limited to, diphtheria A chain, nonbinding active fragments of diphtheria toxin, exotoxin A chain, ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. In some embodiments, the cytotoxic agent is a radioisotope to produce a radioconjugate or a radioconjugated agent. A variety of radionuclides are available for the production of radioconjugated agents including, but not limited to, 90Y, 125I, 131I, 123I, 111In, 131In, 105Rh, 153Sm, 67Cu, 67Ga, 166Ho, 177Lu, 186Re, 188Re, and 212Bi. Conjugates of a polypeptide or molecule and one or more small molecule toxins, such as a calicheamicin, maytansinoids, a trichothene, and CC1065, and the derivatives of these toxins that have toxin activity, can also be used. Conjugates of a polypeptide or molecule and cytotoxic agent are made using a variety of bifunctional protein-coupling agents such as N-succinimidyl-3-(2-pyridyidithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene).

In some embodiments, a multispecific binding agent (e.g., an antibody, such as a bispecific antibody), including a multispecific binding agent that binds to B7H3, including human B7H3, and GD2, including human GD2, described herein is conjugated to a drug such as a signal transduction modulator, a pro-apoptotic agent, a mitotic inhibitor, an anti-tumor antibiotic, an immunomodulating agent, a nucleic acid for gene therapy, an alkylating agent, an anti-angiogenic agent, an anti-metabolite, a boron-containing agent, a chemoprotective agent, a hormone agent, an anti-hormone agent, a corticosteroid, a photoactive therapeutic agent, an oligonucleotide, a radionuclide agent, a radiosensitizer, a topoisomerase inhibitor, and a tyrosine kinase inhibitor. In some embodiments, the mitotic inhibitor is a dolastatin, an auristatin, a maytansinoid, and a plant alkaloid. In some embodiments, the drug is a dolastatin, an auristatin, a maytansinoid, and a plant alkaloid. An example of an auristatin is monomethylaurisatin F (MMAF) or monomethyauristatin E (MMAE). Examples of maytansinoids include, but are not limited to, DM1, DM2, DM3, and DM4. In some embodiments, the anti-tumor antibiotic is selected from the group consisting of an actinomycine, an anthracycline, a calicheamicin, and a duocarmycin. In some embodiments, the actinomycine is a pyrrolobenzodiazepine (PBD).

Multispecific binding agents (e.g., antibodies, such as bispecific antibodies), including multispecific binding agents that bind to B7H3, including human B7H3, and GD2, including human GD2, described herein may be bispecific, trispecific or of greater multispecificity. Such agents may include antibodies. Multispecific antibodies, such as bispecific antibodies, are monoclonal antibodies that have binding specificities for at least two different targets (e.g., antigens) or two different epitopes on the same target (e.g., a bispecific antibody directed to B7H3 with a first binding domain for a first epitope of a B7H3, and a second binding domain for a second epitope of B7H3). In some embodiments, the first binding domain of multispecific (e.g., bispecific) antibodies described herein can be constructed based on the sequences of the antibodies described herein, e.g., the CDR sequences listed in Table 1. In some embodiments, the second binding domain of multispecific (e.g., bispecific) antibodies described herein can be constructed based on the sequences of the antibodies described herein, e.g., the CDR sequences listed in Table 2. In some embodiments, the multispecific antibodies described herein are bispecific antibodies. In some embodiments, bispecific antibodies are mouse, chimeric, human or humanized antibodies.

Multispecific binding agents (e.g., antibodies, such as bispecific antibodies), including multispecific binding agents that bind to B7H3, including human B7H3, and GD2, including human GD2, described herein can be afucosylated. The glycoylsation status of a multispecific binding agent (e.g., an antibody, such as a bispecific antibody) described herein can modify the activity (e.g., ADCC), pharmacokinetics, and pharmacodynamics of the multispecific binding agent. For example, effector function supporting the cytotoxicity of a multispecific binding agent (e.g., an antibody, such as a bispecific antibody) described herein can be enhanced by decreasing the level of fucose sugars (e.g., fucosylation levels) present on a multispecific binding agent, such as in the Fc portion of a bispecific antibody. Accordingly, in some embodiments, a multispecific binding agent (e.g., an antibody, such as a bispecific antibody) can include a glycan having reduced or no fucose sugar units. In some embodiments, the glycan does not include one or more fucose sugar units. In some embodiments, the glycan having reduced or no fucose sugar units is located in an Fc region of an antibody (e.g., bispecifc antibody).

Methods for generating afucosylated polypeptides are well known in the art, any one of which can be used to generate a multispecific binding agent (e.g., an antibody, such as a bispecific antibody) that is afucosylated. For example, a multispecific binding agent (e.g., an antibody, such as a bispecific antibody) can be produced using a genetically engineered cell line with absent or reduced fucosylation capacity, such as using an interfering RNA for inhibition of alpha-(1,6)-fucosyltransferase expression, which interferes with translation of the FUT8 mRNA, or use of commercially available cell lines, such as POTELLIGENT CHOK1 SV host cell lines (Lonza) and GLYMAXX host cell lines (ProBiogen), or those described in U.S. Pat. Nos. 11,267,899 and 11,168,143. Afucosylated polypeptides many also be made by manipulating the manufacturing process, for example by controlling osmolarity and/or using enzyme inhibitors, such as the methods described in EP2480671.

Multispecific binding agents (e.g., antibodies, such as bispecific antibodies), including multispecific binding agents that bind to B7H3, including human B7H3, and GD2, including human GD2, described herein can have specific binding, functional and/or pharmacokinetics, as described herein. For example, in some embodiments, a multispecific binding agent (e.g., an antibody, such as a bispecific antibody) described herein has a higher avidity to a cell co-expressing B7H3 and GD2 as compared to its avidity to a cell that expresses GD2, but does not express B7H3. Such increased avidity can be at least 10-fold, at least 20-fold, at least 50-fold, at least 100-fold, at least 200-fold, at least 300-fold, at least 400-fold, at least 500-fold, at least 600-fold, at least 700-fold, at least 800-fold, at least 900-fold, at least 10,000-fold, at least 20,000-fold, or at least 50,000-fold higher avidity to a cell co-expressing B7H3 and GD2 as compared to its avidity to a cell that expresses GD2, but does not express B7H3. As another example, in some embodiments, a multispecific binding agent (e.g., an antibody, such as a bispecific antibody) described herein has greater antibody-dependent cell-mediated cytotoxicity (ADCC) activity against a cell co-expressing B7H3 and GD2 as compared to ADCC activity against a cell that expresses GD2, but does not express B7H3. Such increased ADCC activity can be at least 2-fold, at least 3-fold, at least 4-fold, or at least 5-fold higher for a cell co-expressing B7H3 and GD2 as compared to its ADCC activity to a cell that expresses GD2, but does not express B7H3. As yet another example, in some embodiments, a multispecific binding agent (e.g., an antibody, such as a bispecific antibody) described herein has a pharmacokinetic profile that includes one or more of the following: (a) a half-life of at least 5, at least 6, at least 7, least 8, at least 9, or at least 10 days following administration to a subject (e.g., intravenous bolus administration of 5 mg/kg); (b) the multispecific binding agent reaches a maximum serum concentration (Tₘₐₓ) at about 5 hours, at about 6 hours, at about 7 hours, at about 8 hours, at about 9 hours, at about 10 hours, at about 11 hours, at about 12 hours, by less than 12 hours, by less than 11 hours, by less than 10 hours, by less than 9 hours, by less than 8 hours, by less than 7 hours, by less than 6 hours, or by less than 5 hours following administration to a subject (e.g., intravenous bolus administration of 5 mg/kg); and (c) the multispecific binding agent reaches a maximum plasma concentration (Cₘₐₓ) of at least 50,000 ng/mL, at least 100,000 ng/mL, at least 150,000 ng/mL, at least 200,000 ng/mL, at least 250,000 ng/mL, or at least 300,000 ng/mL following administration to a subject (e.g., intravenous bolus administration of 5 mg/kg). As yet a still further example, in some embodiments, a multispecific binding agent (e.g., an antibody, such as a bispecific antibody) described herein has low immunogenicity, which is comparable to antibodies known to have low immunogenicity, such a trastuzumab. As yet a still further example, in some embodiments, a multispecific binding agent (e.g., an antibody, such as a bispecific antibody) described herein has, once purified, demonstrates low aggregation, but high stability in solution as compared to monoclonal antibodies having comparable antigen binding regions (mono-specific or bispecific), but unmodified IgG constant regions (CL, CH1, CH2, and CH3).

Measurement of the properties of the multispecific binding agent (e.g., an antibody, such as a bispecific antibody) as described here can be measured using methods well known in the art, including those described herein, such as those found in Example 2 (e.g., affinity or avidity), Example 3 (e.g., ADCC activity), Example 4 (e.g., pharmacokinetics), Example 5 (e.g., immunogenicity), and Example 6 (e.g., production and developability).

Methods for making multispecific antibodies are known in the art, such as, by co-expression of two immunoglobulin heavy chain-light chain pairs, where the two heavy chains have different specificities (see, e.g., Milstein and Cuello, 1983, Nature 305:537-40). For further details of generating multispecific antibodies (e.g., bispecific antibodies), see, for example, Bispecific Antibodies (Kontermann ed., 2011).

Exemplary structures of multispecific antibodies are known in the art and are further described in Weidle et al., 2013, Cancer Genomics & Proteomics 10: 1-18; Brinkman et al., 2017, MABS, 9:2, 182-212; Godar et al., 2018, Expert Opinion on Therapeutic Patents, 28:3, 251-276; and Spiess et al., 2015, Mol. Immunol. 67 95-106.

For example, bispecific antibody molecules can be classified into different structural groups: (i) bispecific immunoglobulin G (BsIgG); (ii) IgG appended with an additional antigen-binding moiety; (iii) bispecific antibody fragments; (iv) bispecific fusion proteins; and (v) bispecific antibody conjugates. As a non-limiting example, BsIgG formats can include crossMab, DAF (two-in-one), DAF (four-in-one), DutaMab, DT-IgG, knobs-in-holes common LC, knobs-in-holes assembly, charge pair, Fab-arm exchange, SEEDbody, triomab, LUZ-Y, Fcab, _{K}λ-body, orthogonal Fab.

In some embodiments, BsIgG comprises heavy chains that are engineered for heterodimerization. For example, heavy chains can be engineered for heterodimerization using a “knobs-into-holes” strategy, a SEED platform, a common heavy chain (e.g., in KA-bodies), and use of heterodimeric Fc regions. Strategies are known in the art to avoid heavy chain pairing of homodimers in BsIgG, including knobs-into-holes, duobody, azymetric, charge pair, HA-TF, SEEDbody, and differential protein A affinity.

Another bispecific antibody format is IgG appended with an additional antigen-binding moiety. For example, monospecific IgG can be engineered to have bispecificity by appending an additional antigen-binding unit onto the monospecific IgG, e.g., at the N- or C-terminus of either the heavy or light chain. Exemplary additional antigen-binding units include single domain antibodies (e.g., variable heavy chain or variable light chain), engineered protein scaffolds, and paired antibody variable domains (e.g., single chain variable fragments or variable fragments). Non-limiting examples of appended IgG formats include dual variable domain IgG (DVD-Ig), IgG(H)-scFv, scFv-(H)IgG, IgG(L)-scFv, scFv-(L)IgG, IgG(L,H)-Fv, IgG(H)-V, V(H)—IgG, IgG(L)-V, V(L)-IgG, KIH IgG-scFab, 2scFv-IgG, IgG-2scFv, scFv4-Ig, zybody, and DVI-IgG (four-in-one). See Spiess et al. Mol. Immunol. 67(2015):95-106. In some embodiments, an exemplary antibody format is a B-Body format for monospecific or multispecific (e.g., bispecific antibodies) as described in e.g., International Patent Application Publication No. WO 2018/075692 and US Patent Application Publication No. 2018/0118811.

Bispecific antibody fragments are a format of bispecific antibody molecules that lack some or all of the antibody constant domains. For example, some bispecific antibody fragments lack an Fc region. In embodiments, bispecific antibody fragments include heavy and light chain regions that are connected by a peptide linker that permits efficient expression of the bispecific antibody fragments in a single host cell. Non-limiting examples of bispecific antibody fragments include, but are not limited to, nanobody, nanobody-HAS, BiTE, Diabody, DART, TandAb, scDiabody, scDiabody-CH3, Diabody-CH3, triple body, miniantibody, minibody, TriBi minibody, scFv-CH3 KIH, Fab-scFv, scFv-CH-CL-scFv, F(ab′)2, F(ab′)2-scFv2, scFv-KIH, Fab-scFv-Fc, tetravalent HCAb, scDiabody-Fc, Diabody-Fc, tandem scFv-Fc, and intrabody.

Bispecific fusion proteins include antibody fragments linked to other proteins. For example, bispecific fusion proteins can be linked to other proteins to add additional specificity and/or functionality. In some embodiments, the dock-and-lock (DNL) method can be used to generate bispecific antibody molecules with higher valency. For example, bispecific antibody fusions to albumin binding proteins or human serum albumin can be extend the serum half-life of antibody fragments. In embodiments, chemical conjugation, e.g., chemical conjugation of antibodies and/or antibody fragments, can be used to create bispecific antibody fragments molecules. An exemplary bispecific antibody conjugate includes the CovX-body format, in which a low molecular weight drug is conjugated site-specifically to a single reactive lysine in each Fab arm or an antibody or fragment thereof. In embodiments, the conjugation improves the serum half-life.

Methods of production of multispecific antibodies, including bispecific antibodies, are known in the art. For example, multispecific antibodies, including bispecific antibodies, can be produced by separate expression of the component antibodies in different host cells and subsequent purification/assembly or by expression of the component antibodies in a single host cell. Purification of multispecific (e.g., bispecific) antibody molecules can be performed by various methods known in the art, including affinity chromatography.

In some embodiments, multispecific binding agents (e.g., antibodies, such as bispecific antibodies), including multispecific binding agents that bind to B7H3, including human B7H3, and GD2, including human GD2, disclosed herein can be provided in any antibody format disclosed herein or known in the art. As a non-limiting example, in some embodiments, the multispecific binding agents (e.g., antibodies, such as bispecific antibodies), including multispecific binding agents that bind to B7H3, including human B7H3, and GD2, including human GD2, can be selected from Fabs-in-tandem-Ig (FIT-Ig); DVD-Ig; hybrid hybridoma (quadroma or tetradoma); anticalin platform (Pieris); diabodies; single chain diabodies; tandem single chain Fv fragments; TandAbs, Trispecific Abs (Affimed); Darts dual affinity retargeting (Macrogenics); Bispecific Xmabs (Xencor); Bispecific T cell engagers (Bites; Amgen; 55 kDa); Triplebodies; Tribody=Fab-scFv Fusion Protein multifunctional recombinant antibody derivates (CreativeBiolabs); Duobody platform (Genmab); dock and lock platform; knobs-into-holes (KIH) platform; Humanized bispecific IgG antibody (REGN1979) (Regeneron); Mab2 bispecific antibodies (F-Star); DVD-Ig=dual variable domain immunoglobulin (Abbott); kappa-lambda bodies; TBTI=tetravalent bispecific tandem Ig; and CrossMab (Roche).

In some embodiments, a multispecific (e.g., bispecific) antibody disclosed herein comprises a B7H3 binding domain and a GD2 binding. In some embodiments, a multispecific (e.g., bispecific) antibody disclosed herein comprises a B7H3 binding domain that comprises the VH and/or VL amino acid sequences of Table 1.

In some embodiments, described herein is a multispecific (e.g., bispecific) antibody comprising a binding domain which binds to B7H3 that comprises VH and VL CDRs as set forth in Table 1.

In some embodiments, a multispecific (e.g., bispecific) antibody disclosed herein comprises a B7H3 binding domain and GD2 binding domain. In some embodiments, a multispecific (e.g., bispecific) antibody disclosed herein comprises a B7H3 binding domain that comprises the VH and/or VL amino acid sequences of Table 1 and a GD2 binding domain that comprises the VH and/or VL amino acid sequences of Table 2.

In some embodiments, a multispecific (e.g., bispecific) antibody disclosed herein comprises a B7H3 binding domain that comprises the VH and VL amino acid sequences of Table 1 and a GD2 binding domain that comprises the VH and VL amino acid sequences of Table 2.

Antibodies that bind B7H3 and/or GD2 may be obtained by any suitable method, such as (but not limited to) immunization with whole tumor cells comprising B7H3 and/or GD2 and collection of antibodies, recombinant techniques, or screening libraries of antibodies or antibody fragments using B7H3 extracellular domain epitopes or GD2 extracellular domain epitopes. Monoclonal antibodies may be generated using a variety of known techniques (see, for example, Coligan et al. (eds.), Current Protocols in Immunology, 1:2.5.12.6.7 (John Wiley & Sons 1991); Monoclonal Antibodies, Hybridomas: A New Dimension in Biological Analyses, Plenum Press, Kennett, McKearn, and Bechtol (eds.) (1980); Antibodies: A Laboratory Manual, Harlow and Lane (eds.), Cold Spring Harbor Laboratory Press (1988); and Picksley et al., “Production of monoclonal antibodies against proteins expressed in E. coli,” in DNA Cloning 2: Expression Systems, 2nd Edition, Glover et al. (eds.), page 93 (Oxford University Press 1995)). One exemplary technique for generating monoclonal antibodies comprises immunizing an animal with a human B7H3 antigen and generating a hybridoma from spleen cells taken from the animal. A hybridoma may produce a monoclonal antibody or antibody fragment that binds B7H3. One exemplary technique for generating monoclonal antibodies comprises immunizing an animal with a human GD2 antigen and generating a hybridoma from spleen cells taken from the animal. A hybridoma may produce a monoclonal antibody or antibody fragment that binds GD2.

In a further embodiment, monoclonal antibodies or antibody fragments can be isolated from antibody phage libraries generated using the techniques described in, for example, Antibody Phage Display: Methods and Protocols, P. M. O'Brien and R. Aitken, eds, Humana Press, Totawa N.J., 2002. In principle, synthetic antibody clones are selected by screening phage libraries containing phage that display various fragments of antibody variable region (Fv) fused to phage coat protein. Such phage libraries are screened for against the desired antigen. Clones expressing Fv fragments capable of binding to the desired antigen are adsorbed to the antigen and thus separated from the non-binding clones in the library. The binding clones are then eluted from the antigen, and can be further enriched by additional cycles of antigen adsorption/elution.

Variable domains can be displayed functionally on phage, either as single-chain Fv (scFv) fragments, in which VH and VL are covalently linked through a short, flexible peptide, or as Fab fragments, in which they are each fused to a constant domain and interact non-covalently, as described, for example, in Winter et al., Ann. Rev. Immunol., 12: 433-455 (1994).

Repertoires of VH and VL genes can be separately cloned by polymerase chain reaction (PCR) and recombined randomly in phage libraries, which can then be searched for antigen-binding clones as described in Winter et al., supra. Libraries from immunized sources provide high-affinity antibodies to the immunogen without the requirement of constructing hybridomas. Alternatively, the naive repertoire can be cloned to provide a single source of human antibodies to a wide range of non-self and also self antigens without any immunization as described by Griffiths et al., EMBO J, 12: 725-734 (1993). Finally, naive libraries can also be made synthetically by cloning the unrearranged V-gene segments from stem cells, and using PCR primers containing random sequence to encode the highly variable CDR3 regions and to accomplish rearrangement in vitro as described, for example, by Hoogenboom and Winter, J. Mol. Biol., 227: 381-388 (1992).

Screening of the libraries can be accomplished by various techniques known in the art. For example, B7H3 (e.g., a B7H3 polypeptide, fragment or epitope) or GD2 (e.g., a GD2 polypeptide, fragment or epitope) can be used to coat the wells of adsorption plates, expressed on host cells affixed to adsorption plates or used in cell sorting, or conjugated to biotin for capture with streptavidin-coated beads, or used in any other method for panning display libraries. The selection of antibodies with slow dissociation kinetics (e.g., good binding affinities) can be promoted by use of long washes and monovalent phage display as described in Bass et al., Proteins, 8: 309-314 (1990) and in WO 92/09690, and a low coating density of antigen as described in Marks et al., Biotechnol., 10: 779-783 (1992).

Multispecific binding agents can be obtained by designing a suitable antigen screening procedure to select for the phage clone of interest followed by construction of a full length multispecific binding agent (e.g., an antibody, such as a bispecific antibody) clone using VH and/or VL sequences (e.g., the Fv sequences), or various CDR sequences from VH and VL sequences, from the phage clone of interest and suitable constant region (e.g., Fc) sequences described in Kabat et al., Sequences of Proteins of Immunological Interest, Fifth Edition, NIH Publication 91-3242, Bethesda MD (1991), vols. 1-3.

Likewise, human antibodies that bind B7H3 and/or GD2 may be generated by any of a number of techniques including, but not limited to, Epstein Barr Virus (EBV) transformation of human peripheral blood cells (e.g., containing B lymphocytes), in vitro immunization of human B cells, fusion of spleen cells from immunized transgenic mice carrying inserted human immunoglobulin genes, isolation from human immunoglobulin V region phage libraries, or other procedures as known in the art and based on the disclosure herein. Methods for obtaining human antibodies from transgenic animals are further described, for example, in Bruggemann et al., Curr. Opin. Biotechnol., 8: 455 58, 1997; Jakobovits et al., Ann. N.Y. Acad. Sci., 764: 525 35, 1995; Green et al., Nature Genet., 7: 13-21, 1994; Lonberg et al., Nature, 368: 856-859, 1994; Taylor et al., Int. Immun. 6: 579-591, 1994; and U.S. Pat. No. 5,877,397.

For example, human antibodies that bind B7H3 and/or GD2 may be obtained from transgenic animals that have been engineered to produce specific human antibodies in response to antigenic challenge. For example, International Patent Publication No. WO 98/24893 discloses transgenic animals having a human Ig locus, wherein the animals do not produce functional endogenous immunoglobulins due to the inactivation of endogenous heavy and light chain loci. Transgenic non-primate mammalian hosts capable of mounting an immune response to an immunogen, wherein the antibodies have primate constant and/or variable regions, and wherein the endogenous immunoglobulin encoding loci are substituted or inactivated, also have been described. International Patent Publication No. WO 96/30498 discloses the use of the Cre/Lox system to modify the immunoglobulin locus in a mammal, such as to replace all or a portion of the constant or variable region to form a modified antibody molecule. International Patent Publication No. WO 94/02602 discloses non-human mammalian hosts having inactivated endogenous Ig loci and functional human Ig loci. U.S. Pat. No. 5,939,598 discloses methods of making transgenic mice in which the mice lack endogenous heavy chains, and express an exogenous immunoglobulin locus comprising one or more xenogeneic constant regions. Using a transgenic animal, such as a transgenic animal described herein, an immune response can be produced to a selected antigenic molecule, and antibody producing cells can be removed from the animal and used to produce hybridomas that secrete human-derived monoclonal antibodies. Immunization protocols, adjuvants, and the like are known in the art, and are used in immunization of, for example, a transgenic mouse as described in International Patent Publication No. WO 96/33735. The monoclonal antibodies can be tested for the ability to inhibit or neutralize the biological activity or physiological effect of the corresponding protein.

The present disclosure provides humanized antibodies that bind B7H3, including human B7H3, and GD2, including human GD2. Humanized antibodies of the present disclosure may comprise a first binding domain that binds to B7H3 and comprises one or more CDRs as shown in Table 1. Humanized antibodies of the present disclosure may comprise a second binding domain that binds to GD2 and comprises one or more CDRs as shown in Table 2. Various methods for humanizing non-human antibodies are known in the art. For example, a humanized antibody can have one or more amino acid residues introduced into it from a source that is non-human. These non-human amino acid residues are often referred to as “import” residues, which are typically taken from an “import” variable domain. Humanized antibodies that bind B7H3 may be produced using techniques known to those skilled in the art (Zhang et al., Molecular Immunology, 42(12): 1445-1451, 2005; Hwang et al., Methods, 36(1): 35-42, 2005; Dall'Acqua et al., Methods, 36(1): 43-60, 2005; Clark, Immunology Today, 21(8): 397-402, 2000, and U.S. Pat. Nos. 6,180,370; 6,054,927; 5,869,619; 5,861,155; 5,712,120; and 4,816,567, all of which are all hereby expressly incorporated herein by reference).

In some cases, the humanized antibodies are constructed by CDR grafting, in which the amino acid sequences of the six complementarity determining regions (CDRs) of the parent non-human antibody (e.g., rodent) are grafted onto a human antibody framework. For example, Padlan et al. (FASEB J. 9:133-139, 1995) determined that only about one third of the residues in the CDRs actually contact the antigen, and termed these the “specificity determining residues,” or SDRs. In the technique of SDR grafting, only the SDR residues are grafted onto the human antibody framework (see, e.g., Kashmiri et al., Methods 36: 25-34, 2005).

The choice of human variable domains, both light and heavy, to be used in making the humanized antibodies can be important to reduce antigenicity. For example, according to the so-called “best-fit” method, the sequence of the variable domain of a non-human (e.g., rodent) antibody is screened against the entire library of known human variable-domain sequences. The human sequence which is closest to that of the rodent may be selected as the human framework for the humanized antibody (Sims et al. (1993) J. Immunol. 151:2296; Chothia et al. (1987) J. Mol. Biol. 196:901. Another method uses a particular framework derived from the consensus sequence of all human antibodies of a particular subgroup of light or heavy chains. The same framework may be used for several different humanized antibodies (Carter et al. (1992) Proc. Natl. Acad. Sci. USA, 89:4285; Presta et al. (1993) J. Immunol., 151:2623. In some cases, the framework is derived from the consensus sequences of the most abundant human subclasses, V_{L}6 subgroup I (V_{L}6I) and V_{H} subgroup III (V_{H}III). In another method, human germline genes are used at the source of the framework regions.

In an alternative paradigm based on comparison of CDRs, called Superhumanization, FR homology is irrelevant. The method consists of comparison of the non-human sequence with the functional human germline gene repertoire. Those genes encoding the same or closely related canonical structures to the murine sequences are then selected. Next, within the genes sharing the canonical structures with the non-human antibody, those with highest homology within the CDRs are chosen as FR donors. Finally, the non-human CDRs are grafted onto these FRs (see, e.g., Tan et al., J. Immunol. 169: 1119-1125, 2002).

It is further generally desirable that antibodies be humanized with retention of their affinity for the antigen and other favorable biological properties. To achieve this goal, according to one method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. These include, for example, WAM (Whitelegg and Rees, Protein Eng. 13: 819-824, 2000), Modeller (Sali and Blundell, J. Mol. Biol. 234: 779-815, 1993), and Swiss PDB Viewer (Guex and Peitsch, Electrophoresis 18: 2714-2713, 1997). Inspection of these displays permits analysis of the likely role of the residues in the functioning of the candidate immunoglobulin sequence, e.g., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

Another method for antibody humanization is based on a metric of antibody humanness termed Human String Content (HSC). This method compares the mouse sequence with the repertoire of human germline genes and the differences are scored as HSC. The target sequence is then humanized by maximizing its HSC rather than using a global identity measure to generate multiple diverse humanized variants. (Lazar et al., Mol. Immunol. 44: 1986-1998, 2007).

In addition to the methods described above, empirical methods may be used to generate and select humanized antibodies. These methods include those that are based upon the generation of large libraries of humanized variants and selection of the best clones using enrichment technologies or high throughput screening techniques. Antibody variants may be isolated from phage, ribosome and yeast display libraries as well as by bacterial colony screening (see, e.g., Hoogenboom, Nat. Biotechnol. 23: 1105-1116, 2005; Dufner et al., Trends Biotechnol. 24: 523-529, 2006; Feldhaus et al., Nat. Biotechnol. 21: 163-70, 2003; Schlapschy et al., Protein Eng. Des. Sel. 17: 847-60, 2004).

In the FR library approach, a collection of residue variants are introduced at specific positions in the FR followed by selection of the library to select the FR that best supports the grafted CDR. The residues to be substituted may include some or all of the “Vernier” residues identified as potentially contributing to CDR structure (see, e.g., Foote and Winter, J. Mol. Biol. 224: 487-499, 1992), or from the more limited set of target residues identified by Baca et al. (J. Biol. Chem. 272: 10678-10684, 1997).

In FR shuffling, whole FRs are combined with the non-human CDRs instead of creating combinatorial libraries of selected residue variants (see, e.g., Dall'Acqua et al., Methods 36: 43-60, 2005). The libraries may be screened for binding in a two-step selection process, first humanizing VL, followed by VH. Alternatively, a one-step FR shuffling process may be used. Such a process has been shown to be more efficient than the two-step screening, as the resulting antibodies exhibited improved biochemical and physico-chemical properties including enhanced expression, increased affinity and thermal stability (see, e.g., Damschroder et al., Mol. Immunol. 44: 3049-60, 2007).

The “humaneering” method is based on experimental identification of essential minimum specificity determinants (MSDs) and is based on sequential replacement of non-human fragments into libraries of human FRs and assessment of binding. It begins with regions of the CDR3 of non-human VH and VL chains and progressively replaces other regions of the non-human antibody into the human FRs, including the CDR1 and CDR2 of both VH and VL. This methodology typically results in epitope retention and identification of antibodies from multiple sub-classes with distinct human V-segment CDRs. Humaneering allows for isolation of antibodies that are 91-96% homologous to human germline gene antibodies. (See, e.g., Alfenito, Cambridge Healthtech Institute's Third Annual PEGS, The Protein Engineering Summit, 2007).

The “human engineering” method involves altering an non-human antibody or antibody fragment, such as a mouse or chimeric antibody or antibody fragment, by making specific changes to the amino acid sequence of the antibody so as to produce a modified antibody with reduced immunogenicity in a human that nonetheless retains the desirable binding properties of the original non-human antibodies. Generally, the technique involves classifying amino acid residues of a non-human (e.g., mouse) antibody as “low risk”, “moderate risk”, or “high risk” residues. The classification is performed using a global risk/reward calculation that evaluates the predicted benefits of making particular substitution (e.g., for immunogenicity in humans) against the risk that the substitution will affect the resulting antibody's folding and/or are substituted with human residues. The particular human amino acid residue to be substituted at a given position (e.g., low or moderate risk) of a non-human (e.g., mouse) antibody sequence can be selected by aligning an amino acid sequence from the non-human antibody's variable regions with the corresponding region of a specific or consensus human antibody sequence. The amino acid residues at low or moderate risk positions in the non-human sequence can be substituted for the corresponding residues in the human antibody sequence according to the alignment. Techniques for making human engineered proteins are described in greater detail in Studnicka et al., Protein Engineering, 7: 805-814 (1994), U.S. Pat. Nos. 5,766,886, 5,770,196, 5,821,123, and 5,869,619, and PCT Application Publication WO 93/11794.

In some embodiments, a multispecific binding agent described herein comprises a non-antibody protein scaffold. Non-limiting examples of such a non-antibody protein scaffold include a fibronectin scaffold, an anticalin, an adnectin, an affibody, a DARPin, a fynomer, an affitin, an affilin, an avimer, a cysteine-rich knottin peptide, or an engineered Kunitz-type inhibitor. Methods for generating such non-antibody protein scaffolds are well known in the art, any one of which can be used to generate a multispecific binding agent comprising a non-antibody protein scaffold (see, e.g., Simeon and Chen, Protein Cell, 9(1):3-14 (2018); Yang et al., Annu Rev Anal Chem (Palo Alto Calif). 10(1):293-320 (2017)).

Further provided are the materials for generating mutispecific binding agents and fragments thereof. For example, an isolated cell (e.g., a hybridoma) may produce a multispecific binding agent (e.g., antibody or antibody fragment). In this regard, a cell (e.g., an isolated cell) may produce an antibody or fragment thereof comprising a first binding domain comprising a VH and a VL as shown in Table 1 for mAb-B7H3. Alternatively or additionally, a cell (e.g., an isolated cell) may produce an antibody or fragment thereof comprising a second binding domain comprising a VH and a VL as shown in Table 2 for mAb-GD2. In some embodiments, polynucleotides described herein may comprise one or more nucleic acid sequences encoding the multispecific binding agent (e.g., antibody or antibody fragment). In some embodiments, the polynucleotide is an isolated and/or recombinant polynucleotide. In various aspects, the isolated polynucleotide comprises a nucleotide sequence that encodes an antibody heavy chain variable region (VH) and/or an antibody light chain variable region (V_{L}), wherein the V_{H} and the V_{L} comprise complementarity determining regions (CDRs) identical to CDRs as shown in Table 1. In various aspects, the isolated polynucleotide comprises a nucleotide sequence that encodes an antibody heavy chain variable region (VH) and/or an antibody light chain variable region (V_{L}), wherein the V_{H} and the V_{L} comprise complementarity determining regions (CDRs) identical to CDRs as shown in Table 2.

In some embodiments, one or more vectors (e.g., expression vectors) may comprise one or more polynucleotides for expression of the one or more polynucleotides in a suitable host cell. Such vectors are useful, e.g., for amplifying the polynucleotides in host cells to create useful quantities thereof, and for expressing binding agents, such as antibodies or antibody fragments, using recombinant techniques.

In some embodiments, one or more vectors are expression vectors wherein one or more polynucleotides are operatively linked to one or more polynucleotides comprising expression control sequences. Autonomously replicating recombinant expression constructs such as plasmid and viral DNA vectors incorporating one or more polynucleotides encoding antibody sequences that bind B7H3 are specifically contemplated. Expression control DNA sequences include promoters, enhancers, and operators, and are generally selected based on the expression systems in which the expression construct is to be utilized. Promoter and enhancer sequences are generally selected for the ability to increase gene expression, while operator sequences are generally selected for the ability to regulate gene expression. Expression constructs may also include sequences encoding one or more selectable markers that permit identification of host cells bearing the construct. Expression constructs may also include sequences that facilitate, and preferably promote, homologous recombination in a host cell. In some embodiments, expression constructs of the can also include sequences necessary for replication in a host cell.

Exemplary expression control sequences include promoter/enhancer sequences, e.g., cytomegalovirus promoter/enhancer (Lehner et al., J. Clin. Microbiol., 29: 2494-2502, 1991; Boshart et al., Cell, 41: 521-530, 1985); Rous sarcoma virus promoter (Davis et al., Hum. Gene Ther., 4: 151, 1993); Tie promoter (Korhonen et al., Blood, 86(5): 1828-1835, 1995); simian virus 40 promoter; DRA (downregulated in adenoma; Alrefai et al., Am. J. Physiol. Gastrointest. Liver Physiol., 293: G923-G934, 2007); MCT1 (monocarboxylate transporter 1; Cuff et al., Am. J. Physiol. Gastrointet. Liver Physiol., G977-G979. 2005); and Math1 (mouse atonal homolog 1; Shroyer et al., Gastroenterology, 132: 2477-2478, 2007), for expression in mammalian cells, the promoter being operatively linked upstream (e.g., 5′) of a polypeptide coding sequence. In another variation, the promoter is an epithelial-specific promoter or endothelial-specific promoter. Polynucleotides may also optionally include a suitable polyadenylation sequence (e.g., the SV40 or human growth hormone gene polyadenylation sequence) operably linked downstream (e.g., 3′) of the polypeptide coding sequence.

If desired, the one or more polynucleotides also optionally comprise nucleotide sequences encoding secretory signal peptides fused in frame with the polypeptide sequences. The secretory signal peptides direct secretion of the antibody polypeptides by the cells that express the one or more polynucleotides, and are cleaved by the cell from the secreted polypeptides. The one or more polynucleotides may further optionally comprise sequences who's only intended function is to facilitate large scale production of the vector. One can manufacture and administer polynucleotides for gene therapy using procedures that have been described in the literature for a variety of transgenes. See, e.g., Isner et al., Circulation, 91: 2687-2692, 1995; and Isner et al., Human Gene Therapy, 7: 989-1011, 1996.

In some embodiments, polynucleotides may further comprise additional sequences to facilitate uptake by host cells and expression of the antibody (and/or any other peptide). In some embodiments, a “naked” transgene encoding an antibody described herein (e.g., a transgene without a viral, liposomal, or other vector to facilitate transfection) is employed.

Any suitable vectors may be used to introduce one or more polynucleotides that encode an antibody into the host. Exemplary vectors that have been described include replication deficient retroviral vectors, including but not limited to lentivirus vectors (Kim et al., J. Virol., 72(1): 811-816, 1998; Kingsman & Johnson, Scrip Magazine, October, 1998, pp. 43-46); parvoviral vectors, such as adeno-associated viral (AAV) vectors (U.S. Pat. Nos. 5,474,935; 5,139,941; 5,622,856; 5,658,776; 5,773,289; 5,789,390; 5,834,441; 5,863,541; 5,851,521; 5,252,479; Gnatenko et al., J. Invest. Med., 45: 87-98, 1997); adenoviral (AV) vectors (U.S. Pat. Nos. 5,792,453; 5,824,544; 5,707,618; 5,693,509; 5,670,488; 5,585,362; Quantin et al., Proc. Natl. Acad. Sci. USA, 89: 2581-2584, 1992; Stratford Perricaudet et al., J. Clin. Invest., 90: 626-630, 1992; and Rosenfeld et al., Cell, 68: 143-155, 1992); an adenoviral adeno-associated viral chimeric (U.S. Pat. No. 5,856,152) or a vaccinia viral or a herpesviral vector (U.S. Pat. Nos. 5,879,934; 5,849,571; 5,830,727; 5,661,033; 5,328,688); Lipofectin mediated gene transfer (BRL); liposomal vectors (U.S. Pat. No. 5,631,237); and combinations thereof. Any of these expression vectors can be prepared using standard recombinant DNA techniques described in, e.g., Sambrook et al., Molecular Cloning, a Laboratory Manual, 2d edition, Cold Spring Harbor Press, Cold Spring Harbor, N.Y. (1989), and Ausubel et al., Current Protocols in Molecular Biology, Greene Publishing Associates and John Wiley & Sons, New York, N.Y. (1994). Optionally, viral vectors are rendered replication-deficient by, e.g., deleting or disrupting select genes required for viral replication.

Other non-viral delivery mechanisms contemplated include calcium phosphate precipitation (Graham and Van Der Eb, Virology, 52: 456-467, 1973; Chen and Okayama, Mol. Cell Biol., 7: 2745-2752, 1987; Rippe et al., Mol. Cell Biol., 10: 689-695, 1990) DEAE-dextran (Gopal, Mol. Cell Biol., 5: 1188-1190, 1985), electroporation (Tur-Kaspa et al., Mol. Cell Biol., 6: 716-718, 1986; Potter et al., Proc. Nat. Acad. Sci. USA, 81: 7161-7165, 1984), direct microinjection (Harland and Weintraub, J. Cell Biol., 101: 1094-1099, 1985, DNA-loaded liposomes (Nicolau and Sene, Biochim. Biophys. Acta, 721: 185-190, 1982; Fraley et al., Proc. Natl. Acad. Sci. USA, 76: 3348-3352, 1979; Felgner, Sci Am., 276(6): 102-6, 1997; Felgner, Hum Gene Ther., 7(15): 1791-3, 1996), cell sonication (Fechheimer et al., Proc. Natl. Acad. Sci. USA, 84: 8463-8467, 1987), gene bombardment using high velocity microprojectiles (Yang et al., Proc. Natl. Acad. Sci USA, 87: 9568-9572, 1990), and receptor-mediated transfection (Wu and Wu, J. Biol. Chem., 262: 4429-4432, 1987; Wu and Wu, Biochemistry, 27: 887-892, 1988; Wu and Wu, Adv. Drug Delivery Rev., 12: 159-167, 1993).

An expression vector (or the antibody discussed herein) may be entrapped in a liposome. See, e.g., Ghosh and Bachhawat, In: Liver diseases, targeted diagnosis and therapy using specific receptors and ligands, Wu G, Wu C ed., New York: Marcel Dekker, pp. 87-104 (1991); Radler et al., Science, 275(5301): 810-814, 1997). Also contemplated are various commercial approaches involving “lipofection” technology. In some embodiments, the liposome may be complexed with a hemagglutinating virus (HVJ). This has been shown to facilitate fusion with the cell membrane and promote cell entry of liposome-encapsulated DNA (Kaneda et al., Science, 243: 375-378, 1989). In some embodiments, the liposome is complexed or employed in conjunction with nuclear nonhistone chromosomal proteins (HMG-1) (Kato et al., J. Biol. Chem., 266: 3361-3364, 1991). In some embodiments, the liposome are complexed or employed in conjunction with both HVJ and HMG-1. Such expression constructs have been successfully employed in transfer and expression of nucleic acid in vitro and in vivo. In some embodiments, a multispecific binding agent (e.g., an antibody, such as a bispecific antibody), including a multispecific binding agent that binds to B7H3, including human B7H3, and GD2, including human GD2, is included in the liposome to target the liposome to cells (such as tumor cells) expressing B7H3 and/or GD2 on their surface.

A cell may comprise one or more polynucleotides or one or more vectors, e.g., the cell is transformed or transfected with one or more polynucleotides encoding a multispecific binding agent (e.g., an antibody, such as a bispecific antibody), including a multispecific binding agent that binds to B7H3, including human B7H3, and GD2, including human GD2, or the one or more vectors comprising the one or more polynucleotides. In some embodiments, cells express a multispecific binding agent, including a multispecific binding agent that binds to B7H3, including human B7H3, and GD2, including human GD2, having at least 75% identity to the CDRs of mAb-B7H3 (see, e.g., Table 1). In some embodiments, cells express a multispecific binding agent, including a multispecific binding agent that binds to B7H3, including human B7H3, and GD2, including human GD2, having at least 75% identity to the CDRs of mAb-GD2 (see, e.g., Table 2). The cells may be prokaryotic cells, such as Escherichia coli (see, e.g., Pluckthun et al., Methods Enzymol., 178: 497-515, 1989), or eukaryotic cells, such as an animal cell (e.g., a myeloma cell, Chinese Hamster Ovary (CHO) cell, or hybridoma cell), yeast (e.g., Saccharomyces cerevisiae), or a plant cell (e.g., a tobacco, corn, soybean, or rice cell). Use of mammalian host cells may provide for translational modifications (e.g., glycosylation, truncation, lipidation, and phosphorylation) that may be desirable to confer optimal biological activity on recombinant expression products. Similarly, polypeptides (e.g., multispecific binding agents (e.g., antibodies, such as bispecific antibodies), including multispecific binding agents that bind to B7H3, including human B7H3, and GD2, including human GD2) may be glycosylated or non-glycosylated and/or have been covalently modified to include one or more water soluble polymer attachments such as polyethylene glycol, polyoxyethylene glycol, or polypropylene glycol.

Methods for introducing DNA or RNA into host cells are well known and include transformation, transfection, electroporation, nuclear injection, or fusion with carriers such as liposomes, micelles, ghost cells, and protoplasts. Such host cells are useful for amplifying polynucleotides and also for expressing polypeptides encoded by the polynucleotides. In this regard, a process for the production of a multispecific binding agent (e.g., an antibody, such as a bispecific antibody) may comprise culturing a host cell and isolating the multispecific binding agent. Transferring a naked DNA expression construct into cells can be accomplished using particle bombardment, which depends on the ability to accelerate DNA coated microprojectiles to a high velocity allowing them to pierce cell membranes and enter cells without killing them (Klein et al., Nature, 327: 70-73, 1987). Several devices for accelerating small particles have been developed. One such device relies on a high voltage discharge to generate an electrical current, which in turn provides the motive force (Yang et al., Proc. Natl. Acad. Sci USA, 87: 9568-9572, 1990). The microprojectiles used have consisted of biologically inert substances such as tungsten or gold beads. A host cell may be isolated and/or purified. A host cell also may be a cell transformed in vivo to cause transient or permanent expression of the polypeptide in vivo. A host cell may also be an isolated cell transformed ex vivo and introduced post-transformation, e.g., to produce the polypeptide in vivo for therapeutic purposes. The definition of host cell explicitly excludes a transgenic human being.

In some aspects, provided herein are methods of generating a multispecific binding agent (e.g., an antibody, such as a bispecific antibody) as described here. Such methods can include culturing a cell having one or more vectors that include one or more polynucleotides encoding a multispecific binding agent (e.g., an antibody, such as a bispecific antibody) as described here in a culture medium to produce the multispecific binding agent in the culture medium. In some embodiments, such methods can further include isolating the multispecific binding agent (e.g., an antibody, such as a bispecific antibody) from the culture medium, and purifying the multispecific binding agent (e.g., an antibody, such as a bispecific antibody) isolated from the culture medium.

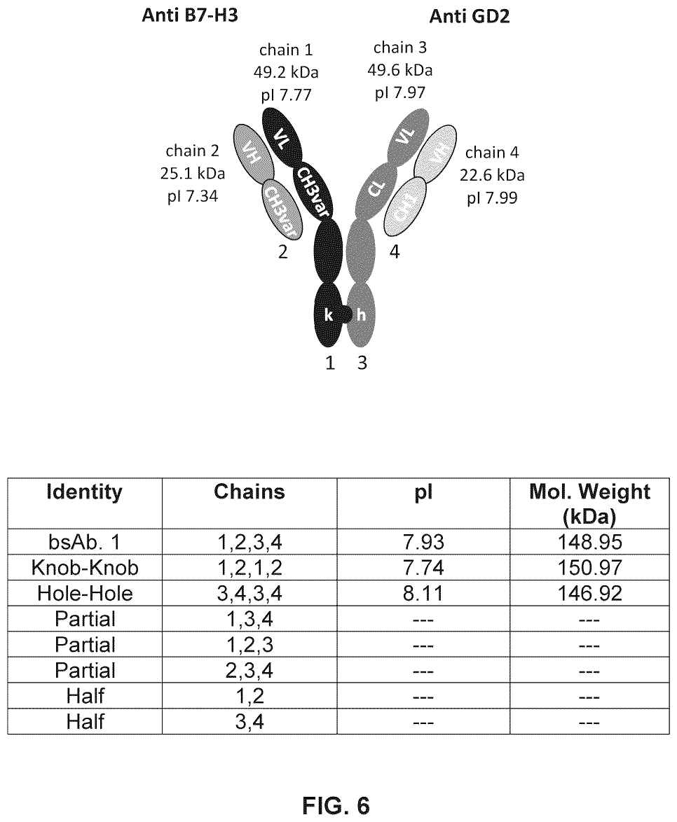

Performance of such methods generates a composition having a multispecific binding agent (e.g., an antibody, such as a bispecific antibody) as described herein. Such a composition produced by these methods has significantly higher yields of properly formed multispecific binding agent (e.g., an antibody, such as a bispecific antibody), with a significantly lower amount of improperly formed complexes, as described herein in FIG. 6. For example, a properly formed bispecific antibody having four polypeptide chains as described herein includes a first polypeptide chain (chain 1), a second polypeptide chain (chain 2), a third polypeptide chain (chain 3), and a four polypeptide chain (chain 4), but an improperly formed complex includes complexes having: chains 1, 2, 1, 2; chains 3, 4, 3, 4; chains 1, 3, 4; chains 1, 2, 3; chains 2, 3, 4; chains 1, 2; or chains 3, 4. By performing the methods of generating a multispecific binding agent (e.g., an antibody, such as a bispecific antibody) as described herein, compositions having the multispecific binding agent (e.g., an antibody, such as a bispecific antibody) can include at least 75%, at least 80%, at least 85%, at least 90%, at least 95%, at least 98%, at least 99%, or at least 99.9 pure properly formed multispecific binding agent. Additionally, such compositions can include a concentration of properly formed multispecific binding agent of at least 1,500 mg/L, at least 2,000 mg/L, at least 2,500 mg/L, at least 3,500 mg/L, at least 4,000 mg/L, at least 4,500 mg/L, at least 5,000 mg/L, at least 5,500 mg/L, or at least 6,000 mg/L with at least 10 days of culturing, at least 2,000 mg/L, at least 2,500 mg/L, at least 3,500 mg/L, at least 4,000 mg/L, at least 4,500 mg/L, at least 5,000 mg/L, at least 5,500 mg/L, at least 6,000 mg/L, or at least 6,500 mg/L with at least 12 days of culturing, or at least 2,500 mg/L, at least 3,500 mg/L, at least 4,000 mg/L, at least 4,500 mg/L, at least 5,000 mg/L, at least 5,500 mg/L, at least 6,000 mg/L, at least 6,500 mg/L, at least 7,000 mg/L, or at least 7,500 mg/L with at least 14 days of culturing. Alternatively, such compositions can include a concentration of properly formed multispecific binding agent of at least 1,500 mg/L, at least 2,000 mg/L, at least 2,500 mg/L, at least 3,500 mg/L, at least 4,000 mg/L, at least 4,500 mg/L, at least 5,000 mg/L, at least 5,500 mg/L, or at least 6,000 mg/L with no more than 10 days of culturing, at least 2,000 mg/L, at least 2,500 mg/L, at least 3,500 mg/L, at least 4,000 mg/L, at least 4,500 mg/L, at least 5,000 mg/L, at least 5,500 mg/L, at least 6,000 mg/L, or at least 6,500 mg/L with no more than 12 days of culturing, or at least 2,500 mg/L, at least 3,500 mg/L, at least 4,000 mg/L, at least 4,500 mg/L, at least 5,000 mg/L, at least 5,500 mg/L, at least 6,000 mg/L, at least 6,500 mg/L, at least 7,000 mg/L, or at least 7,500 mg/L with no more than 14 days of culturing.

A variety of methods for producing antibodies from polynucleotides are generally well-known. For example, basic molecular biology procedures are described by Maniatis et al., Molecular Cloning, A Laboratory Manual, 2nd ed., Cold Spring Harbor Laboratory, New York, 1989 (see also Maniatis et al, 3rd ed., Cold Spring Harbor Laboratory, New York, 2001). Additionally, numerous publications describe techniques suitable for the preparation of antibodies by manipulation of DNA, creation of expression vectors, and transformation and culture of appropriate cells (see, e.g., Mountain and Adair, Chapter 1 in Biotechnology and Genetic Engineering Reviews, Tombs ed., Intercept, Andover, U K, 1992); and Current Protocols in Molecular Biology, Ausubel ed., Wiley Interscience, New York, 1999).

A multispecific binding agent (e.g., an antibody, such as a bispecific antibody), including a multispecific binding agent that binds to B7H3, including human B7H3, and GD2, including human GD2, is produced using any suitable method, e.g., isolated from a culture medium, an immunized animal, recombinantly or synthetically generated, or genetically-engineered, including as described above. In some embodiments, producing the multispecific binding agent (e.g., an antibody, such as a bispecific antibody) includes isolating the multispecific binding agent from a composition containing the multispecific binding agent, such as a culture medium, includes contacting the culture medium with a resin for binding to the multispecific binding agent (e.g., a protein A or anti-CH1 based resin); and eluting the multispecific binding agent from the resin. In some embodiments, producing the multispecific binding agent (e.g., an antibody, such as a bispecific antibody) includes purifying a composition containing the multispecific binding agent, which can include contacting the eluted bispecific antibody from an isolation resin with a purification resin, such an ion exchange resin (e.g., cation exchange resin); and eluting the bispecific antibody from the purification resin.

A multispecific binding agent (e.g., an antibody, such as a bispecific antibody), including a multispecific binding agent that binds to B7H3, including human B7H3, and GD2, including human GD2, can be genetically engineered. For example, a multispecific binding agent (e.g., an antibody, such as a bispecific antibody), including a multispecific binding agent that binds to B7H3, including human B7H3, and GD2, including human GD2 comprises, for example, a variable region generated by recombinant DNA engineering techniques. In this regard, a variable region is optionally modified by insertions, deletions, or changes in the amino acid sequence of the antibody to produce an antibody of interest, including as described above. Polynucleotides encoding complementarity determining regions (CDRs) of interest are prepared, for example, by using polymerase chain reaction to synthesize variable regions using mRNA of antibody producing cells as a template (see, for example, Courtenay Luck, “Genetic Manipulation of Monoclonal Antibodies,” in Monoclonal Antibodies: Production, Engineering and Clinical Application, Ritter et al. (eds.), page 166 (Cambridge University Press 1995); Ward et al., “Genetic Manipulation and Expression of Antibodies,” in Monoclonal Antibodies: Principles and Applications, Birch et al., (eds.), page 137 (Wiley Liss, Inc. 1995); and Larrick et al., Methods: A Companion to Methods in Enzymology, 2: 106-110, 1991). Current antibody manipulation techniques allow construction of engineered variable regions containing at least one CDR and, optionally, one or more framework amino acids from a first antibody and the remainder of the variable region from a second antibody. Such techniques are used, e.g., to humanize an antibody or to improve its affinity for a binding target.

“Humanized antibodies” are antibodies in which CDRs of heavy and light variable chains of non-human immunoglobulins are transferred into a human variable region. Constant regions need not be present, but if they are, they optionally are substantially identical to human immunoglobulin constant regions, e.g., at least about 85-90%, about 95%, 96%, 97%, 98%, 99% or more identical, in some embodiments. Hence, in some instances, all parts of a humanized immunoglobulin, except possibly the CDRs, are substantially identical to corresponding parts of natural human immunoglobulin sequences. For example, humanized antibodies are human immunoglobulins (e.g., host antibody) in which hypervariable region residues of the host antibody are replaced by hypervariable region residues from a non-human species (donor antibody) such as mouse, rat, rabbit, or a non-human primate having the desired specificity, affinity, and capacity.

In some embodiments, multispecific binding agents (e.g., antibodies), including multispecific binding agents that binds to B7H3, including human B7H3, and GD2, including human GD2 described herein are useful in compositions and in methods of treating, preventing, or alleviating a cancer, including one or more symptoms of the cancer. Such a cancer includes, but is not limited to, any cancer wherein the cancer cell expresses or overexpresses B7H3 and/or GD2. Such B7H3 and/or GD2 expressing cancer cells may help cancer cells escape immune surveillance and clearance (e.g., tumor immunity) or contribute to cancer cell resistance to anti-cancer drugs. For example, B7H3 is understood to be either a co-stimulatory molecule for T cell activation or play a nonimmunological role in regulating signaling pathways. Additionally, GD2 is highly expressed on neuroectoderm-derived tumors and sarcomas, and GD2 has been shown to enhance tumor cell proliferation and invasiveness, including in small cell lung cancer and osteosarcoma cells. In addition, multispecific binding agents described herein, such as multispecific antibodies (e.g., antibodies, such as bispecific antibodies), that bind to B7H3 and GD2, are useful to selectively target and enhance removal of cancer cells.

In some embodiments, described herein is a method for treating or preventing a cancer in a subject comprising administering to the subject a multispecific binding agent (e.g., an antibody, such as a bispecific antibody), including a multispecific binding agent that binds to B7H3, including human B7H3, and GD2, including human GD2, described herein or a pharmaceutical composition comprising the multispecific binding agent (e.g., an antibody, such as a bispecific antibody) described herein. For example, the method comprises administering an amount of a multispecific binding agent (e.g., a bispecific antibody), such as a multispecific binding agent that binds to B7H3, including human B7H3, and GD2, including human GD2 described herein effective to inhibit proliferation of cancer cells or to remove cancer cells. In some embodiments, the method includes administering a multispecific binding agent (e.g., a bispecific antibody) that includes an antigen binding site that binds B7H3 (see, e.g., CDRs and VH/VL of Table 1) and an antigen binding site that binds GD2 (see, e.g., CDRs and VH/VL of Table 2) resulting in the inhibition of the proliferation of cancer or tumor cells or to remove cancer or tumor cells. In some embodiments, the method includes administering a multispecific binding agent (e.g., a bispecific antibody) that includes an antigen binding site that binds B7H3 (see, e.g., CDRs and VH/VL of Table 1) and an antigen binding site that binds GD2 (see, e.g., CDRs and VH/VL of Table 2), resulting in enhancement of the removal of cancer cells.

In some embodiments, described herein is a method for alleviating one or more symptoms associated with a cancer in a subject comprising administering to the subject a multispecific binding agent (e.g., an antibody, such as a bispecific antibody), including a multispecific binding agent that binds to B7H3, including human B7H3, and GD2, including human GD2, described herein or a pharmaceutical composition comprising the multispecific binding agent (e.g., an antibody, such as a bispecific antibody) described herein.

In some embodiments, described herein is a method for decreasing tumor size in a subject with a tumor comprising administering to the subject a multispecific binding agent (e.g., an antibody, such as a bispecific antibody), including a multispecific binding agent that binds to B7H3, including human B7H3, and GD2, including human GD2, described herein or a pharmaceutical composition comprising the binding agent (e.g., an antibody, such as a bispecific antibody) described herein.

In some embodiments, described herein is a method for enhancing tumor cell removal in a subject with a tumor comprising administering to the subject a multispecific binding agent (e.g., an antibody, such as a bispecific antibody), including a multispecific binding agent that binds to B7H3, including human B7H3, and GD2, including human GD2, described herein or a pharmaceutical composition comprising the multispecific binding agent (e.g., an antibody, such as a bispecific antibody) described herein.

In some embodiments, one or more polynucleotides, vectors, and/or cells as described herein can be used in methods of inhibiting proliferation of cancer cells or to remove cancer cells in vivo (e.g., in a method of treating cancer in a subject).

In some embodiments, described herein is a method selectively binding a cell co-expressing B7 Homolog 3 (B7H3) and disialoganglioside (GD2), such as a cancer cell, in a subject comprising administering to the subject a multispecific binding agent (e.g., an antibody, such as a bispecific antibody), including a multispecific binding agent that binds to B7H3, including human B7H3, and GD2, including human GD2, described herein or a pharmaceutical composition comprising the multispecific binding agent (e.g., an antibody, such as a bispecific antibody) described herein.

The terms “cancer,” “malignancy,” “neoplasm,” “tumor,” “tumour,” and “carcinoma,” are used interchangeably herein to refer to cells that exhibit relatively abnormal, uncontrolled, and/or autonomous growth, so that they exhibit an aberrant growth phenotype characterized by a significant loss of control of cell proliferation. In general, cells of interest for detection or treatment in the present application include precancerous (e.g., benign), malignant, pre-metastatic, metastatic, and non-metastatic cells. The teachings of the present disclosure may be relevant to any and all cancers. To give but a few, non-limiting examples, in some embodiments, teachings of the present disclosure are applied to one or more cancers such as, for example, a brain cancer, an eye cancer, a lung cancer, a skin cancer, and a sarcoma. The multispecific binding agent (e.g., an antibody, such as a bispecific antibody) of the present disclosure can be used for the treatment of B7H3+ and/or GD2+ expressing cancer cells or tumors. Accordingly, cancers and the cells of such cancers that can be targeted using the compositions and methods described herein include, but are not limited to, a brain cancer, an eye cancer, a lung cancer, a skin cancer, and a sarcoma. In some embodiments, the brain cancer is neuroblastoma or glioblastoma. In some embodiments, the eye cancer is a retinoblastoma. In some embodiments, the lung cancer is small cell lung cancer. In some embodiments, the skin cancer is melanoma. In some embodiments, the sarcoma is selected from osteosarcoma, rhabdomyosarcoma, Ewing's sarcoma in children and adolescents, liposarcoma, fibrosarcoma, leiomyosarcoma and other soft tissue sarcomas in adults. Accordingly, specific examples of the types of cancers that can be targeted using the compositions and methods described herein include, but are not limited to, neuroblastoma, glioblastoma, retinoblastoma, small cell lung cancer, melanoma, osteosarcoma, rhabdomyosarcoma, Ewing's sarcoma in children and adolescents, liposarcoma, fibrosarcoma, leiomyosarcoma and other soft tissue sarcomas in adults.

Additionally, the term “solid tumor” refers to an abnormal mass of tissue that usually does not contain cysts or liquid areas. Solid tumors may be benign or malignant. Different types of solid tumors are named for the type of cells that form them. Examples of solid tumors are sarcomas (including cancers arising from transformed cells of mesenchymal origin in tissues such as cancellous bone, cartilage, fat, muscle, vascular, hematopoietic, or fibrous connective tissues), carcinomas (including tumors arising from epithelial cells), melanomas, lymphomas, mesothelioma, neuroblastoma, retinoblastoma, etc. Cancers involving solid tumors include, without limitations, brain cancer, lung cancer, stomach cancer, duodenal cancer, esophagus cancer, breast cancer, colon and rectal cancer, renal cancer, bladder cancer, kidney cancer, pancreatic cancer, prostate cancer, ovarian cancer, melanoma, mouth cancer, sarcoma, eye cancer, thyroid cancer, urethral cancer, vaginal cancer neck cancer, lymphoma, and the like.

In some embodiments, the antibody is a human antibody, including, but not limited to, an antibody having variable regions in which both the framework and CDR regions are derived from human germline immunoglobulin sequences as described, for example, in Kabat et al. (1991) Sequences of proteins of Immunological Interest, Fifth Edition, U.S. Department of Health and Human Services, NIH Publication No. 91-3242. If the antibody contains a constant region, the constant region also preferably is derived from human germline immunoglobulin sequences. Human antibodies may comprise amino acid residues not encoded by human germline immunoglobulin sequences, for example, to enhance the activity of the antibody, but do not comprise CDRs derived from other species (e.g., a mouse CDR placed within a human variable framework region).

A method of modulating (e.g., inhibiting, reducing, preventing) tumor growth in a subject also is provided. For example, the method comprises administering to the subject a composition comprising a multispecific binding agent (e.g., a bispecific antibody) described herein in an amount effective to modulate tumor growth in the subject.

Also provided is a method of treating cancer by administering a multispecific binding agent (e.g., an antibody, such as a bispecific antibody) such as a multispecific binding agent that binds to B7H3, including human B7H3, and GD2, including human GD2, to a subject in need thereof, alone or in combination with another agent. Accordingly, the subject of a method described here can be administered one or more therapeutic agents described herein in combination with a multispecific binding agent (e.g., an antibody, such as a bispecific antibody), including a multispecific binding agent that binds to B7H3, including human B7H3, and GD2, including human GD2, described herein or a pharmaceutical composition comprising the multispecific binding agent (e.g., an antibody, such as a bispecific antibody) described herein.

A particular administration regimen of a multispecific binding agent (e.g., an antibody, such as a bispecific antibody) for a particular subject will depend, in part, upon the agent used, the amount of agent administered, the route of administration, and the cause and extent of any side effects. The amount of agent (e.g., an antibody, such as a bispecific antibody) administered to a subject (e.g., a mammal, such as a human) should be sufficient to affect the desired response over a reasonable time frame. According, in some embodiments, the amount of a multispecific binding agent (e.g., an antibody, such as a bispecific antibody) or pharmaceutical composition described herein administered to a subject is an effective amount. In some embodiments, the amount of a multispecific binding agent (e.g., an antibody, such as a bispecific antibody) or pharmaceutical composition described herein administered to a subject is a therapeutically effective amount. Some conditions or disease states require prolonged treatment, which may or may not entail administering doses of multispecific binding agents (e.g., antibodies, such as bispecific antibodies), including multispecific binding agents that bind to B7H3, including human B7H3, and GD2, including human GD2 (e.g., antibodies, such as bispecific antibodies), over multiple administrations.

Suitable routes of administering a composition comprising a multispecific binding agent (e.g., an antibody, such as a bispecific antibody), such as a multispecific binding agent that binds to B7H3, including human B7H3, and GD2, including human GD2 (e.g., an antibody, such as a bispecific antibody), are well known in the art. Although more than one route can be used to administer a multispecific binding agent (e.g., an antibody, such as a bispecific antibody), a particular route can provide a more immediate and more effective reaction than another route. Depending on the circumstances, a composition comprising a multispecific binding agent (e.g., an antibody, such as a bispecific antibody) such as a multispecific binding agent that binds to B7H3, including human B7H3, and GD2, including human GD2 is applied or instilled into body cavities, absorbed through the skin or mucous membranes, ingested, inhaled, and/or introduced into circulation. For example, it may be desirable to deliver a composition comprising a multispecific binding agent (e.g., an antibody, such as a bispecific antibody) such as a multispecific binding agent that binds to B7H3, including human B7H3, and GD2, including human GD2 through injection by intravenous, subcutaneous, intraperitoneal, intracerebral (intra-parenchymal), intracerebroventricular, intramuscular, intra-ocular, intraarterial, intraportal, intralesional, intramedullary, intrathecal, intraventricular, transdermal, subcutaneous, intraperitoneal, intranasal, enteral, topical, sublingual, urethral, vaginal, or rectal means, by sustained release systems, or by implantation devices. If desired, a multispecific binding agent (e.g., an antibody, such as a bispecific antibody) such as a multispecific binding agent that binds to B7H3, including human B7H3, and GD2, including human GD2 is administered regionally via intraarterial or intravenous administration feeding the region of interest, e.g., via the hepatic artery for delivery to the liver. Alternatively, a multispecific binding agent (e.g., an antibody, such as a bispecific antibody) such as a multispecific binding agent that binds to B7H3, including human B7H3, and GD2, including human GD2 is administered locally via implantation of a membrane, sponge, or another appropriate material on to which the binding agent has been absorbed or encapsulated. Where an implantation device is used, the device is, one aspect, implanted into any suitable tissue or organ, and delivery of a multispecific binding agent (e.g., an antibody, such as a bispecific antibody) such as a multispecific binding agent that binds to B7H3, including human B7H3, and GD2, including human GD2 is, for example, via diffusion, timed-release bolus, or continuous administration. In other aspects, the multispecific binding agent is administered directly to exposed tissue during tumor resection or other surgical procedures.

The present disclosure provides a composition, such as pharmaceutical composition, comprising a multispecific binding agent (e.g., an antibody, such as a bispecific antibody) such as a multispecific binding agent that binds to B7H3, including human B7H3, and GD2, including human GD2 and a carrier (e.g., a pharmaceutically acceptable carrier). The particular carrier employed may depend on chemico-physical considerations, such as solubility and lack of reactivity with the binding agent or co-therapy, and by the route of administration. Pharmaceutically acceptable carriers are well-known in the art, examples of which are described herein. Illustrative pharmaceutical forms suitable for injectable use include sterile aqueous solutions or dispersions and sterile powders for the extemporaneous preparation of sterile injectable solutions or dispersions. Injectable formulations are further described in, e.g., Pharmaceutics and Pharmacy Practice, J. B. Lippincott Co., Philadelphia. Pa., Banker and Chalmers. eds., pages 238-250 (1982), and ASHP Handbook on Injectable Drugs, Toissel, 4th ed., pages 622-630 (1986)). A pharmaceutical composition comprising a multispecific binding agent (e.g., an antibody, such as a bispecific antibody) such as a multispecific binding agent that binds to B7H3, including human B7H3, and GD2, including human GD2 is, in one aspect, placed within containers, along with packaging material that provides instructions regarding the use of such pharmaceutical compositions. Generally, such instructions include a tangible expression describing the reagent concentration, as well as, in some embodiments, relative amounts of excipient ingredients or diluents (e.g., water, saline or PBS) that may be necessary to reconstitute the pharmaceutical composition.

In some aspects, a method described herein further comprises administering one or more additional agents, including therapeutic agents (e.g., combination therapy), which may be present in a composition or may be administered with a multispecific binding agent (e.g., an antibody, such as a bispecific antibody) such as a multispecific binding agent that binds to B7H3, including human B7H3, and GD2, including human GD2 or provided in a separate composition using the same or a different route of administration. The one or more additional agents, including therapeutic agents, may be administered together or separately (e.g., simultaneously, alternatively, sequentially) with a multispecific binding agent (e.g., an antibody, such as a bispecific antibody). Such additional therapeutic agents include, but are not limited to, an anticancer drug (e.g., a chemotherapeutic agent), radiotherapy (by applying irradiation externally to the body or by administering radio conjugated compounds), an anti-tumor antigen or marker antibody (the antigen or marker being for example CD4, CD38, CA125, PSMA, c-MET, VEGF, CD137, VEGFR2, CD20, HER2, HER3, SLAMF7, CD326, CAIX, CD40, CD47, or EGF receptor), a checkpoint inhibitor or an immunomodulating antibody (for example an antibody targeting PD-1, PD-LI, TIM3, CD38, GITR, CD134, CD134L, CD137, CD137L, CDS0, CD86, B7-H3, B7-H4, B7RP1, LAG3, ICOS, TIM3, GAL9, CD28, AP2MI, SHP-2, OX-40, VISTA, TIGIT, BTLA, HVEM, CD160, etc.), a vaccine (in particular, a cancer vaccine, for example GVAX), an inhibitor, a cytoxic agent, an adjuvant, standard-of-use protocol, one or more other compounds targeting cancer cells or stimulating an immune response against cancer cells, or any combination thereof. In some embodiments, the additional agent is an immune check point inhibitor, such as a PD-1 antagonist, for example an anti-PD1 antibody or an anti-PDL1 antibody. In certain embodiments, when such one or more additional agent or therapy is or comprises an antibody, the format of and/or the antigen targeted by such antibody can be chosen among those listed in the literature and possibly adapted to a given cancer. Such antigens and corresponding antibodies include, but are not limited to, PD-L1 (e.g., Atezolizumab, Avelumab, Durvalumab, Nivolumab, Pembrolizumab), VEGF (e.g., Bevacizumab), PD-1 (e.g., Cemiplimab), EGFR (e.g., Cetuximab, Necitumumab, Panitumumab), CD38 (e.g., Daratumumab, Isatuximab), SLAMF7 (e.g., Elotuzumab), CTLA-4 (e.g., Ipilimumab), CCR4 (e.g., Mogamulizumab), CD20 (e.g., Obinutuzumab, Ofatumumab, Rituximab), PDGFRα (e.g., Olaratumab), HER2 (e.g., Pertuzumab, Trastuzumab), and VEGFR2 (e.g., Ramucirumab). Therapeutic antibodies also include antibody-drug conjugates (ADCs), which can also be used in combination with a multispecific binding agent (e.g., an antibody, such as a bispecific antibody) described herein. Such ADCs include, but are not limited to, Gemtuzumab ozogamicin, Brentuximab vedotin, Trastuzumab emtansine, Inotuzumab ozogamicin, Polatuzumab vedotin, Enfortumab vedotin, Trastuzumab deruxtecan, Sacituzumab govitecan, Moxetumomab pasudotox, Ibritumomab tiuxetan, Iodine (I131) tositumomab, and Blinatumomab.

In some embodiments, when the one or more additional agent or therapy is a chemotherapeutic agent, the chemotherapeutic agent can be any one of those known in the art for use in cancer therapy. Such chemotherapeutic agents includes, without limitation, alkylating agents, anthracyclines, epothilones, nitrosoureas, ethylenimines/methylmelamine, alkyl sulfonates, alkylating agents, antimetabolites, pyrimidine analogs, epipodophylo toxins, enzymes such as L-asparaginase; biological response modifiers such as IFNα, IFN-γ, IL-2, IL-12, G-CSF and GM-CSF; platinum coordination complexes such as cisplatin, oxaliplatin and carboplatin, anthracenediones, substituted urea such as hydroxyurea, methylhydrazine derivatives including N-methylhydrazine (MIH) and procarbazine, adrenocortical suppressants such as mitotane and aminoglutethimide; hormones and antagonists including adrenocorticosteroid antagonists such as prednisone and equivalents, dexamethasone and aminoglutethimide; progestin such as hydroxyprogesterone caproate, medroxyprogesterone acetate and megestrol acetate; estrogen such as diethylstilbestrol and ethinyl estradiol equivalents; antiestrogen such as tamoxifen; androgens including testosterone propionate and fluoxymesterone/equivalents; antiandrogens such as flutamide, gonadotropin-releasing hormone analogs and leuprolide; non-steroidal antiandrogens such as flutamide; molecules that target TGF pathways, IDO (in doleamine deoxigenase), Arginase, and/or CSFIR; PARP inhibitors such as olaparib, Veliparib, Iniparib, Rucaparib; and MET inhibitors such as tivantinib, foretinib, golvatinib, cabozantinib and crizotinib.

Cytotoxic agents that can be used include a substance that inhibits or prevents a cellular function and/or causes cell death or destruction. Exemplary cytotoxic agents include, but are not limited to, radioactive isotopes (e.g., At211, 1131, 1125, Y90, Re186, Re188, Sm153, Bi212, P32, Pb212 and radioactive isotopes of Lu); growth inhibitory agents; enzymes and fragments thereof such as nucleolytic enzymes; and toxins such as small molecule toxins or enzymatically active toxins of bacterial, fungal, plant or animal origin, including fragments and/or variants thereof. Other exemplary cytotoxic agents can be selected from anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine kinase angiogenesis inhibitors, immunotherapeutic agents, proapoptotic agents, inhibitors of LDH-A; inhibitors of fatty acid biosynthesis; cell cycle signaling inhibitors; HDAC inhibitors, proteasome inhibitors; and inhibitors of cancer metabolism.

Inhibitors that can be used include, but are not limited to, kinase inhibitors such as imatinib, baricitinib gefitinib, erlotinib, sorafenib, dasatinib, sunitinib, lapatinib, nilotinib, pirfenidone, pazopanib, crizotinib, vemurafenib, vandetanib, ruxolitinib, axitinib, bosutinib, regorafenib, tofacitinib, cabozantinib, ponatinib, trametinib, dabrafenib, afatinib, ibrutinib, ceritinib, idelalisib, nintedanib, palbociclib, lenvatinib, cobimetinib, abemaciclib, acalabrutinib, alectinib, binimetinib, brigatinib, encorafenib, erdafitinib, everolimus, fostamatinib, gilter, larotrectinib, lorlatinib, netarsudil, osimertinib, pemigatinib, pexidartinib, ribociclib, temsirolimus, XL-092, XL-147, XL-765, XL-499, and XL-880. In some embodiments, a multispecific binding agent (e.g., an antibody, such as a bispecific antibody) as disclosed herein can be combined with one or more inhibitors of the following kinases for the treatment of cancer: Akt1, Akt2, Akt3, TGF-βR, PKA, PKG, PKC, CaM-kinase, phosphorylase kinase, MEKK, ERK, MAPK, mTOR, EGFR, HER2, HER3, HER4, 1NS-R, IGF-1R, IR-R, PDGFαR, PDGFβ/R, CSFIR, KIT, FLK-II, KDR/FLK-1, FLK-4, flt-1, FGFR1, FGFR2, FGFR3, FGFR4, Ron, Sea, TRKA, TRKB, TRKC, FLT3, VEGFR/Flt2, Flt4, EphAI, EphA2, EphA3, EphB2, EphB4, Tie2, Src, Fyn, Lck, Fgr, Btk, Fak, SYR, FRK, JAK (JAK1 and or JAK2), ABL, ALK, CDK7, CDK12, KRAS, and B-Raf.

Additional non-limiting examples of inhibitors that can be combined with a multispecific binding agent (e.g., an antibody, such as a bispecific antibody) of the present disclosure for treatment of cancer include an FGFR inhibitor (FGFR1, FGFR2, FGFR3 or FGFR4, e.g., pemigatinib, an EGFR inhibitor (also known as ErB-1 or HER-1; e.g. erlotinib, gefitinib, vandetanib, orsimertinib, cetuximab, necitumumab, or panitumumab), a VEGFR inhibitor or pathway blocker (e.g., bevacizumab, pazopanib, sunitinib, sorafenib, axitinib, regorafenib, ponatinib, vandetanib, ramucirumab, lenvatinib, ziv-aflibercept), a PARP inhibitor (e.g. olaparib, rucaparib, veliparib or niraparib), a JAK inhibitor (e.g., ruxolitinib, baricitinib, itacitinib), an IDO inhibitor (e.g., epacadostat, NLG919, or BMS-986205, MK7162), an LSD1 inhibitor, a TDO inhibitor, a PI3K-delta inhibitor (e.g., parsaclisib), a PI3K-gamma inhibitor such as PI3K-gamma selective inhibitor, a Pim inhibitor, a CSF1R inhibitor, a TAM receptor tyrosine kinases (Tyro-3, Axl, and Mer), an adenosine receptor antagonist (e.g., A2a/A2b receptor antagonist), an HPK1 inhibitor, a chemokine receptor inhibitor (e.g. CCR2 or CCR5 inhibitor), a SHP1/2 phosphatase inhibitor, a histone deacetylase inhibitor (HDAC) such as an HDAC8 inhibitor, an angiogenesis inhibitor, an interleukin receptor inhibitor, bromo and extra terminal family members inhibitors (for example, bromodomain inhibitors or BET inhibitors, or combinations thereof.

The additional agent may be a pharmaceutically acceptable salt, ester, amide, hydrate, and/or prodrug of any of these therapeutic agents described above or other agents.

Still further, the present disclosure provides a variety of kits or articles of manufacture containing a multispecific binding agent (e.g., an antibody, such as a bispecific antibody) such as a multispecific binding agent that binds to B7H3, including human B7H3, and GD2, including human GD2 as described herein or related compositions that allow the administration, storage, or other use of such a multispecific binding agent. In some embodiments, a provided kit comprises a vessel, syringe, a vial, or other container comprising such compositions, optionally together with one or more articles of manufactures, diluents, reagents, solid phases, and/or instructions for the correct use of the kit.

It is understood that modifications which do not substantially affect the activity of the various embodiments described herein are also provided within the definition of the subject matter described herein. Accordingly, the following examples are intended to illustrate but not limit the present disclosure.

### EXAMPLES

### Example 1. Bispecific Antibody Generation and Binding Specificity

Antibodies to B7H3 and GD2 were generated by either phage display or directed mutagenesis, and bispecific antibodies were constructed from the exemplary antibodies as described herein.

The extracellular domain of human B7H3 was purchased from Acro Biosystems (Cat #B73-H52E2). The non-biotinylated extracellular domain of B7H3 was biotinylated using EZ-Link NHS-PEG12-Biotin (ThermoScientific Cat. No. 21312) using standard protocol as needed. A Fab formatted phage library was used in selections to isolate pools of phage that showed binding to the human B7H3 by polyclonal phage ELISA using standard protocols. Individual phage clones were screened for the ability to bind the biotinylated human B7H3 by monoclonal phage ELISA using standard protocols. The VL and VH regions identified in the phage screen described above were formatted into a bivalent monospecific native human full-length IgG1 structure and expressed via transient transfection in HEK cells and subsequent purification with protein A resin. Purified antibodies were tested for binding to human B7H3 using an Octet (Pall ForteBio) biolayer interferometer.

A murine GD2 antibody was used as a starting point for identification of the GD2 antibody with a reduced affinity for GD2 with a more human framework sequence. Mutations were introduced to humanize the antibody using the IGV3-23 human germline sequence. Mutations were selected based upon structural modeling and homology. After screening candidates for cell binding, further mutations were selected for back-mutation to the original murine sequence to tune binding affinity.

Bispecific (1×1) antibodies were created from the identified B7H3 and GD2 antibodies as depicted in FIG. 1 by reformatting the B7H3 antibodies into the first polypeptide chain (chain 1) and second polypeptide chain (chain 2) of an antibody construct having constant regions of SEQ ID NOS: 69 and 70, respectively and GD2 into the third polypeptide chain (chain 3) and fourth polypeptide chain (chain 4) of an antibody construct having constant regions of SEQ ID NOS: 71 and 72, respectively, transient transfection of the 4 chains into HEK cells, which were cultured in culture medium to produce the antibodies. The bispecific antibodies contained in the cultured medium were subsequently isolated with an anti-CH1 resin followed by polishing using a strong cation exchange resin. Bispecfic constructs in which either the B7H3 or the GD2 arm were replaced with non-binding arms were also created. The resultant B7H3×GD2 and the corresponding B7H3× non-binding arm bispecific antibodies were then tested for cell binding on a human B7H3B78 murine melanoma cell line which overexpressed both human B7H3 and GD2. Candidates showing avidity in the cell binding were advanced to further testing.

To measure binding affinity in B7H3 or GD2 binder discovery campaigns, the 1×1 bispecific antibody was captured to a biosensor on an Octet (Pall ForteBio) biolayer interferometer. Soluble antigens (e.g., B7H3 or GD2) were then added to the system and binding was measured.

Human and cynomolgus orthologs for B7H3 were obtained in proteins of His-tagged format from AcroBiosystems (Human B7H3 (Cat #B73-H82E6) Cyno B7H3 (Cat #B73-C52Ha)), whereas the rat ortholog for B7H3 was obtained from R&D Systems (Recombinant Rat B7-H3 Fc Chimera Protein Cat #10610-B3-050). GD2 was obtained from Biosynth International-Carbosynth (GD2-Oligosaccharide-sp-biotin catalog #0071835).

Exemplary results for the Octet affinity analysis are shown in Table 3. Results indicate that the B7H3 antibody has single digit nM binding affinity to human, cynomolgus and rat B7H3 proteins, whereas the GD2 antibody has significantly weaker binding affinity to human GD2 with greater than triple digit nM binding.

TABLE 3
Octet affinity analysis

| Antigen | kₐ (M⁻¹s⁻¹) | k_{d} (s⁻¹) | Kd (nM) (via kinetics) |
|---|---|---|---|
| Human B7H3 | 2.35E+05 | 2.54E−04 | 1.08 |
| Cynomolgus B7H3 | 2.06E+05 | 2.39E−04 | 1.16 |
| Rat B7H3 | 1.99E+05 | 1.37E−03 | 6.88 |
| GD2 | 3.17E+04 | 6.98E−02 | 2200 |

### Example 2. Bispecific Antibody Cell Binding Assays

The exemplary bispecific antibody (designated Exemplary Bispecific Antibody 1 (Ex. bsAb. 1) herein) was evaluated for binding to multiple cell types, including cells overexpressing B7H3 and GD2, for example, murine melanoma B78 cells, as well as human neuroblastoma cells.

Briefly, the human neuroblastoma cell line CHLA-20 and the murine melanoma cell line B78 overexpressing both human B7H3 and human GD2 were used to assess cellular binding EC50s of antibodies via flow cytometry. Antibodies used in the assessment were Exemplary Bispecific Antibody 1 (Ex. bsAb. 1) or versions of this antibody in which either the GD2 specific arm was replaced with a non-binding arm (B7-H3×Non-Binder) or the B7H3 specific arm was replaced with a non-binding arm (GD2×Non-Binder). Cells were incubated with various concentrations of the indicated antibodies for 1 hour at 4° C. in in a 96-well V-bottom plate followed by washing and incubation with AF488 conjugated Goat Anti-Human IgG Fab Fragment (Jackson Immuno Research) for 30 minutes at 4° C. Following washing and resuspension in cold BD stain buffer, the median fluorescence intensity (MFI) for each sample was determined by flow cytometry and plotted versus concentration of antibody.

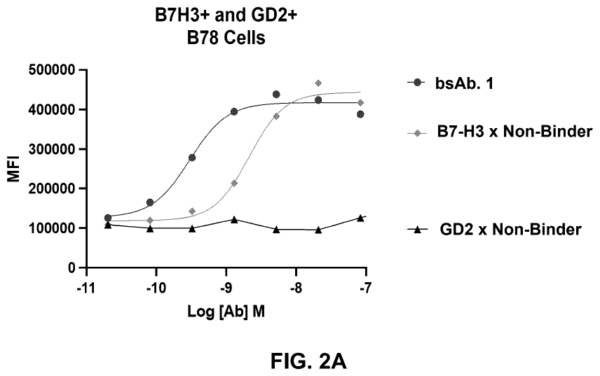

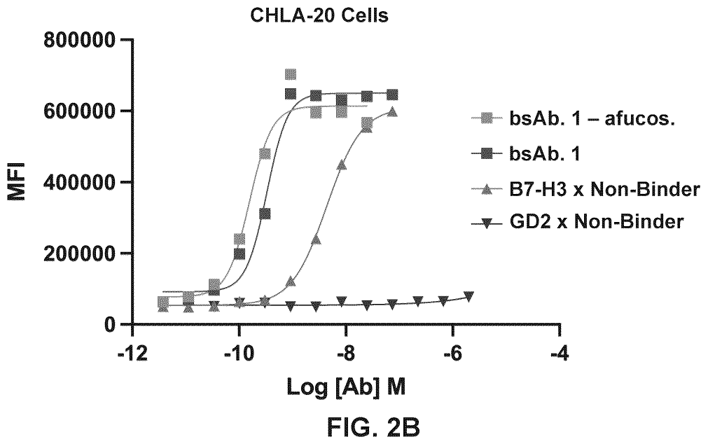

Exemplary binding curves for the B78 cells co-expressing B7H3 and GD2 are depicted in FIG. 2A and for the CHLA-20 cells are depicted in FIG. 2B. Exemplary EC50s values for these binding assays are provided in Table 4. Results indicate that the exemplary bispecific antibody showed significantly higher avidity for cells co-expressing B7H3 and GD2, which was attributable to the GD2 binding arm because the control antibody having only a functional GD2 binding arm (GD2×Non-Binder) showed no observable binding to either type of cell, whereas the control antibody having only a functional B7H3 binding arm (B7-H3×Non-Binder) showed some binding to both cell types. Additionally, the exemplary antibody showed similar binding specificity and avidity to the B78 cells co-expressing B7H3 and GD2 and the human neuroblastoma cells.

TABLE 4
EC50 Values

| | | B7H3 × Non- | GD2 × Non- |
|---|---|---|---|
| Cell Line | Ex. bsAb. 1 | binding arm | binding arm |
| B78 hB7H3+ and | 3.003e−010 | 2.136e−009 | no observable |
| hGD2+ | | | binding) |
| CHLA20 | 7.031e−011 | 3.459e−009 | no observable |
| | | | binding |

Additionally, the exemplary bispecific antibody (designated Exemplary Bispecific Antibody 1 herein) and versions of this antibody in which either the GD2 specific arm or the B7H3 specific arm were replaced with non-binding arms were similarly evaluated for binding to cells expressing only GD2, for example, murine melanoma B78 cells. Results indicate minimal binding to these cells for any of the antibodies tested. Thus, there were no differences in binding between these antibodies to cells expressing only GD2.

These results demonstrate that the bispecific antibodies described herein efficiently and specifically bind to cells co-expressing B7H3 and GD2.

### Example 3. Functional Assays

An exemplary bispecific antibody (designated Exemplary Bispecific Antibody 1 herein) was evaluated for ADCC on a cell type overexpressing B7H3 and GD2, for example, the murine melanoma B78 cells.

Briefly, the ADCC Reporter Bioassay (Promega) was utilized to assess the ADCC potential of a set of antibodies on murine melanoma B78 cells overexpressing both human B7H3 and human GD2, or B78 cells overexpressing human GD2, but not B7H3, following the manufacturers' protocol. Antibodies utilized in this assay were Exemplary Bispecific Antibody 1 (Ex. bsAb. 1), an afucosylated version of Exemplary Bispecific Antibody 1 (Ex. bsAb. 1—afucos.), Exemplary Bispecific Antibody 3 (Ex. bsAb. 3), which contains a silent Fc, versions of Exemplary Bispecific antibody in which either the GD2 specific arm was replaced with a non-binding arm (B7-H3×Non-Binder) or the B7H3 specific arm was replaced with a non-binding arm (GD2×Non-Binder). The afucosylated version of Exemplary Bispecific Antibody 1 was generated by in CHO cells wherein expression of alpha-(1,6)-fucosyltransferase is inhibited by RNA interference of the FUT8 mRNA. Target B78 cells (T) were incubated with various concentration of the indicated antibodies and the Jurkat NFAT effector cells (E) at an E:T ratio of 5:1 for 6 hours at 37° C. in a humidified CO₂ incubator. Following the incubation, the Bio-Glo Luciferase Assay reagent was added, and relative luminescence units (RLU) were determined on a plate reader.

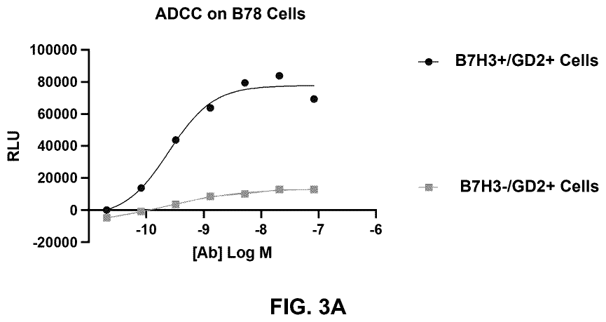

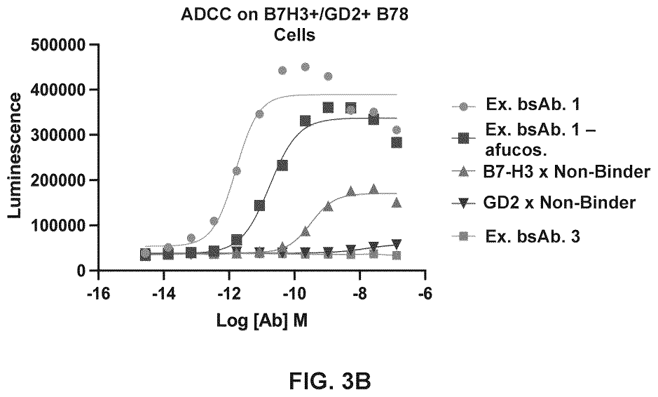

Exemplary results of the ADCC assays are depicted in FIG. 3A and FIG. 3B. Results indicate that the exemplary bispecific antibody showed significant ADCC activity towards cells co-expressing B7H3 and GD2, but little to no ADCC activity on cells expressing only GD2.

Thus, these results demonstrate that the bispecific antibodies described herein efficiently and specifically induced ADCC activity on cells co-expressing B7H3 and GD2, while leaving cells only expressing GD2 unaffected.

### Example 4. Pharmacokinetic Assays

To assess the pharmacokinetic profile of an exemplary bispecific antibody (designated Exemplary Bispecific Antibody 1 herein), adult male Sprague-Dawley rats were administered the exemplary antibody at 5 mg/kg IV bolus. At various timepoints, serum samples were collected and stored at −80° C. until analysis. Concentrations of the exemplary antibody in rat serum samples were determined using the human IgG1 ELISA kit (Cayman Chemical 500910). The concentrations of the exemplary antibody were then used to calculate pharmacokinetic parameters by employing non-compartmental analysis (Phoenix™ WinNonlin).

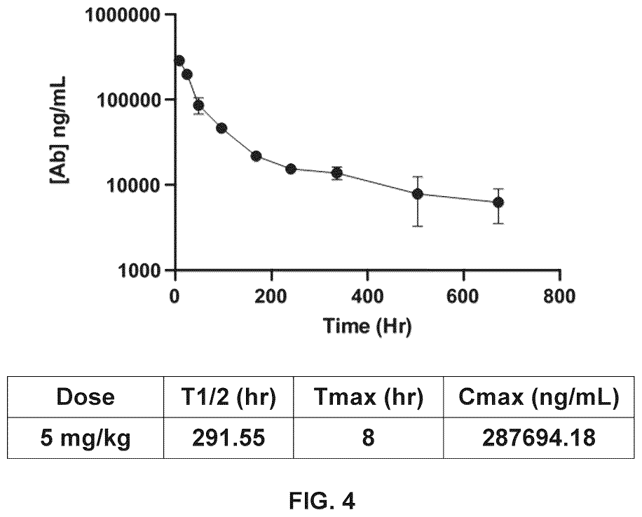

Exemplary results of these analysis are shown in FIG. 4. Results indicate that the half-life of the exemplary antibody in rats is approximately 12 days, reaching a maximum serum concentration (Tₘₐₓ) at about 8 hours, and a maximum plasma concentration (Cₘₐₓ) of 287,694 ng/mL. The exemplary antibody also persisted in the serum for about 28 days at above 1,000 ng/ml.

Thus, these results demonstrate that the pharmacokinetic profiles of the bispecific antibodies described herein are consistent with a monoclonal IgG antibody.

### Example 5. Immunogenicity Assays

To test the immunogenicity of an exemplary bispecific antibody (designated Exemplary Bispecific Antibody 1 herein), a cytokine release assay was performed on the exemplary bispecific antibody and compared to an antibody known to have a low immunogenicity profile.

For these assays, an exemplary bispecific antibody (designated Exemplary Bispecific Antibody 1 herein) or trastuzumab (an antibody with a low immunogenicity profile that does not cause cytokine release in the clinic) were incubated at 4 concentrations (250, 50, 10, and 5 ug/mL) in triplicate with blood from 10 healthy donors. After incubation, plasma was collected and multiplex cytokine analysis was performed for IL-2, IL-6, IL-8, IL-10, IFN-γ, and TNF-α using the Luminex protein analysis system. Statistical analysis was then performed to determine if there was a relevant difference between the exemplary bispecific antibody test sample and trastuzumab. The threshold for a relevant difference was specified based on the variation in outcome for the refence standard (trastuzumab) and for this analysis was determined to be 1.5× root mean square error of a one-way ANOVA with donor as the predictor for the trastuzumab data. This is based on specific guidelines for determining equivalence of products as described in Tsong et al., J. Biopharm. Stat., 27(2):197-205 (2017). The level of each cytokine at the 4 concentrations tested in relation to this equivalence limit were plotted.

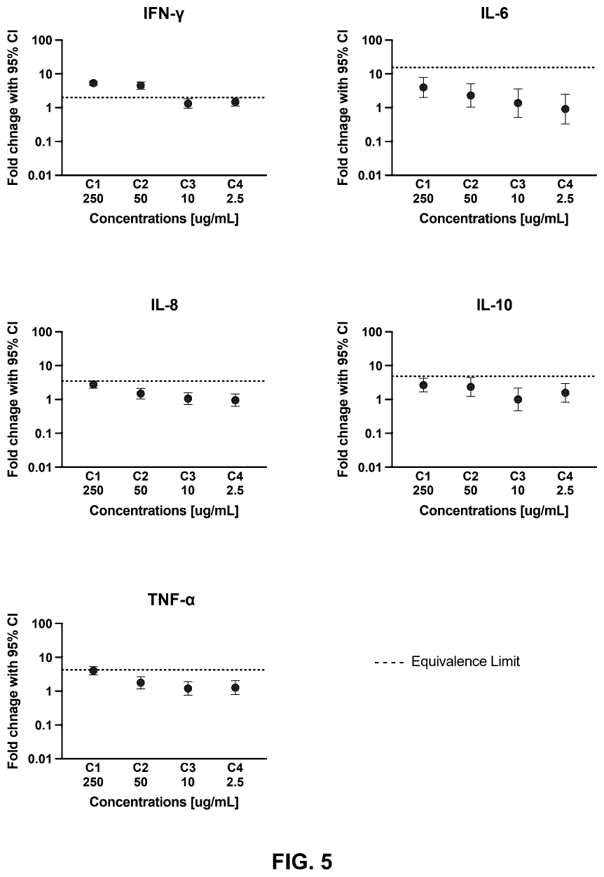

Exemplary results of these assays are shown in FIG. 5. Results indicate that the exemplary bispecific antibody resulted in little to no cytokine release, which was comparable to trastuzumab, which is predictive of little to no immunogenicity in vivo.

### Example 6. Antibody Production and Developability Assays

Various antibody production methods were used to generate, isolate, and purify an exemplary bispecific antibody (designated Exemplary Bispecific Antibody 1 herein), which was tested in various developability methods. For example, various antibody production methods, including cell culture, antibody isolation techniques, including use of protein A or IgG1-CH1 resins, and purification techniques, including polishing by strong cation exchange, were employed. In order to assess developability factors, various methods for assessing bispecific antibodies characteristics, including size exclusion chromatography (SEC), hydrophobic interaction chromatography (HIC), standup monolayer adsorption chromatography (SMAC).

Heavy chain and light chain constructs were transfected into HEK293 or CHO cells using standard protocols. Monoclonal antibodies (mAbs) or bispecifics secreted into the culture media were purified using protein A or CH1 affinity resins

The exemplary bispecific antibody was affinity isolated from the culture medium using protein A type resin or anti-IgG10-CH1 resin. Clarified supernatants generated from 500 mL fed-batch shake flask were affinity purified using CaptureSelect™ CH1-XLAffinity Matrix bulk resin (ThermoFisher PN 194346201L) or MabSelect™ Sure-LX (Cytiva PN 17547403). Resin was hand-packed into columns of 5 cm bed height and run at 1 min residence time, 300 cm/hr using an Akta Pure fast protein liquid chromatography system (FPLC, Cytiva) capable of 25 mL/min flowrate, room temperature. Resin was equilibrated with Dulbecco's PBS (pH 7.4, without Ca2+/Mg2+) prior to loading clarified supernatant, re-equilibrated, washed with DPBS containing 500 mM NaCl, and eluted with a 0.1 M sodium acetate pH 3.5 or other acidic elution buffer. Eluate was immediately neutralized in a dropwise manner with 1 M Tris, pH 9.0. Samples in neutralized elution buffer were dialyzed into DPBS overnight at 4° C. using a dialysis cassette with a 3.5 kDa molecular weight cutoff (Slide-A-Lyzer; ThermoFisher Scientific, PN 66110).

For bispecific antibody preparations, affinity-isolated antibodies from two or more separate lots were pooled prior to polishing or alternatively single lots were used for polishing. Each single or pooled lot was polished by cation exchange chromatography (CEX), such as MonoS 5/50 from Cytiva (0.5 cm×5 cm, 1 mL, 10 μm particle diameter; GE Healthcare Life Sciences, PN17516801), using an AKTA pure fast protein liquid chromatography instrument (FPLC, Cytiva) running Unicorn v7.2 software at a flow rate equivalent to a 2 minute residence time. Aseptic techniques were used at all times. Prior to purification, the column and entire flow-path was sanitized in 1 M NaOH, followed by equilibration with MES pH 6.0. Prior to polishing, the sample was either buffer exchanged into 20 mM MES buffer [pH 6.0, MES=(2-(Nmorpholino)ethanesulfonic acid] or adjusted to match the conductivity and pH of the equilibration buffer. Up to 35 mg was loaded onto the equilibrated column followed by a wash of equilibration buffer to remove unbound sample. Sample was eluted using a linear gradient from 0 to 300 mM NaCl in 20 mM MES pH 6.0 over 20 to 50 CV with fractions collected at 1-mL intervals. Fractions from each major peak were pooled and assessed by non-reducing SDS-PAGE. The pooled sample was buffer exchanged into DPBS as described in the procedure above, sterile filtered through a 0.2 μm filter and stored at 4° C. until analysis.

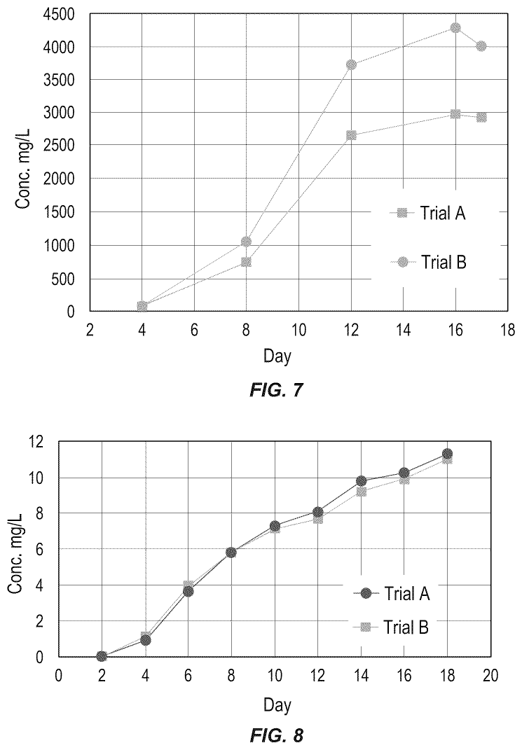

Following the above culturing, isolation, and purification methods, the concentration of the exemplary bispecific antibody was assayed using protein A titer analysis via HPLC, or BioHT, the results of which are depicted in FIG. 7 and FIG. 8, respectively. Results indicate that within 12 days of culture, antibody concentrations reached at least 2,000 mg/L and was as high as about 8,000 mg/L. When the culture was extended to 16 days, antibody concentrations reached about 10,000 mg/L for at least two clonal lots that were assayed.

Size exclusion chromatography (SEC) analysis was performed using a 7.8 mm ID×30 cm TSKgel G3000SWXL column (Tosoh Bioscience LLC, PN 08541) on an Agilent 1100 HPLC. The exemplary bispecific antibody as described herein was normalized to 1 mg/mL concentration in Dulbecco's PBS (pH 7.4, without Ca2+/Mg2+) and clarified via centrifugation to pellet particulates while still retaining soluble aggregates. The mobile phase buffer was Dulbecco's PBS (pH 7.4, without Ca2+/Mg2+). 10 μL sample was loaded and isocratically eluted at 1.0 mL/min over 20 minutes. Absorbance was monitored at 280 nm. Chromatographic peaks were integrated to determine % homogeneity and retention time. The column stationary phase along with choice of mobile phase supports hydrophobic interaction in addition to molecular sizing (hydrophobic interaction much milder compared to SMAC). Data analysis was performed using Agilent ChemStation B.04.03.

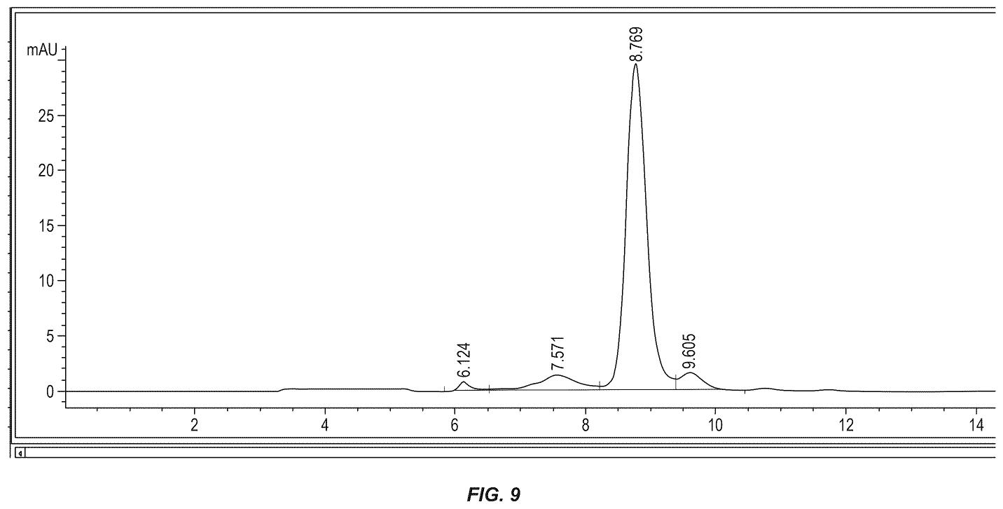

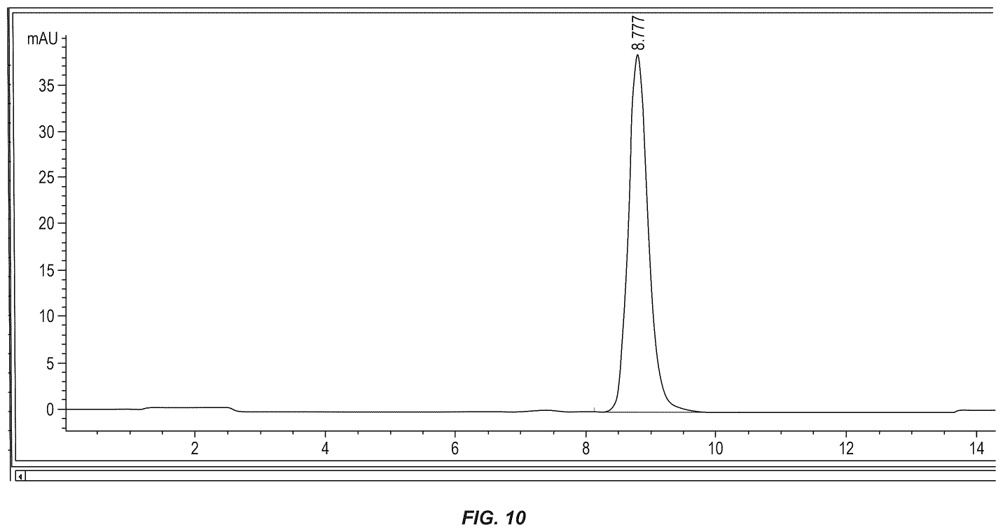

Exemplary SEC results are shown in FIG. 9 and FIG. 10. FIG. 9 shows the SEC results for protein A isolated antibody, whereas as FIG. 10 shows the SEC results for protein A isolated antibody followed by purification with CEX. Results indicate low bispecific antibody aggregation.

Hydrophobic interaction chromatography (HIC) analysis was performed using a 4.6 mm ID×3.5 cm TSKgel Butyl-NPR column (Tosoh Bioscience LLC, PN 14947) on an Agilent 1100 HPLC. The exemplary bispecific antibody as described herein was normalized to 2 mg/mL concentration in dPBS (pH 7.4) and then diluted with an equal volume of mobile phase buffer B to a final protein concentration of 1 mg/mL. The column was equilibrated with 100% mobile phase Buffer B (2 M ammonium sulfate/20 mM sodium phosphate, pH 7.0) at a flow rate of 1 mL/min. 10 μL sample was loaded and eluted using a gradient from 100% mobile phase buffer B to 100% mobile phase buffer A (20 mM sodium phosphate, pH 7.0) at 1.0 mL/min over 15 min, held at 100% A for 3 min to wash the column, and returned 100% B for 2 min for equilibration. Absorbance was monitored at 280 nm. Sample retention time was calculated and compared to a set of standard controls to identify bispecific antibodies with increased retention time (increased hydrophobicity).

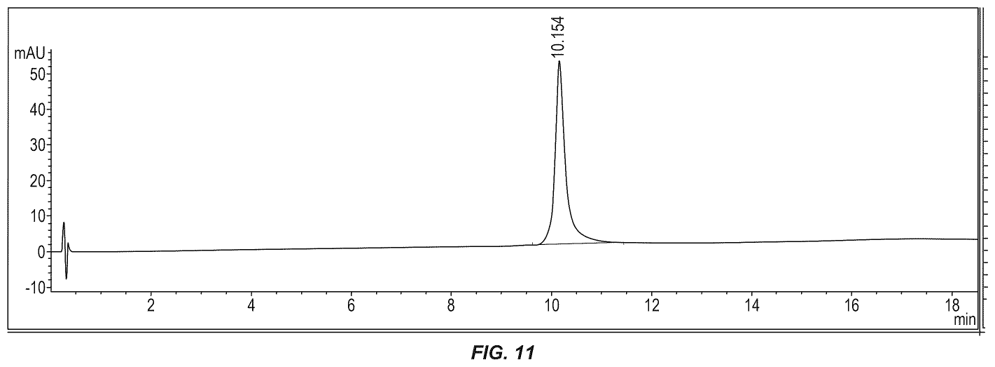

Exemplary HIC results are shown in FIG. 11. Antibody hydrophobicity can impact antibody aggregation, solubility and viscosity. Results show similar retention times for the exemplary bispecific antibody, and indicate a low propensity for aggregation and precipitation. The HIC elution profiles (e.g., sharpness of elution peak and uniform retention times) of the exemplary bispecific antibody suggested low propensities to aggregate and/or precipitate.

Overall, the results of the developability assays show that the developability criteria are met for the exemplary bispecific antibody.

Standup monolayer adsorption chromatography (SMAC) analysis was performed using a 4.6 mm ID×300 mm Zenix SEC 300 column (Sepax Technologies, PN 213300P-4630) on an Agilent 1100 HPLC. The exemplary bispecific antibody as described herein was normalized to 1 mg/mL concentration in dPBS (pH 7.4) and clarified via centrifugation to pellet particulates. The mobile phase buffer was dPBS (pH 7.4, without calcium and magnesium). 10 μL sample was loaded and isocratically eluted at 0.25 mL/min over 32 min. Absorbance was monitored at 280 nm. Sample retention time was calculated and compared to a set of standard controls to identify bispecific antibodies with increased retention time (increased propensity to form aggregates).

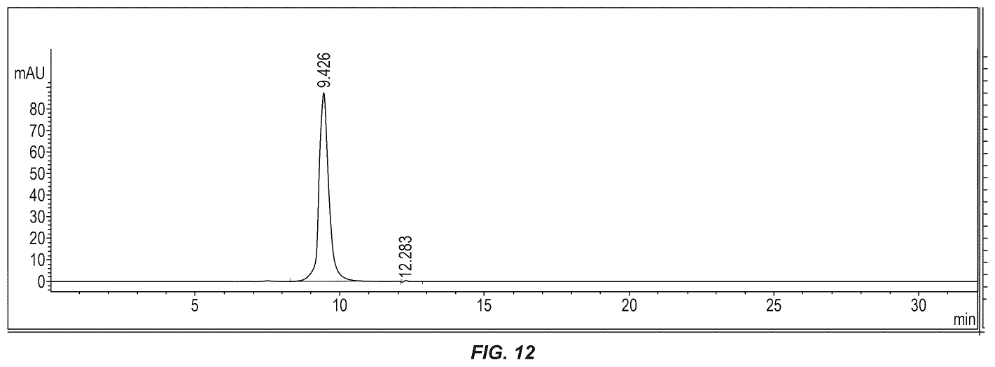

Exemplary SMAC results are shown in FIG. 12. Results show that the exemplary bispecific antibody had similar short retention times, indicating colloidal stability and low propensity to aggregate.

Liquid chromatography mass spectrometry (LC-MS) analysis, including intact Mass Measurements, was also performed on various preparations of the exemplary bispecific antibody. Briefly, intact masses of purified antibodies were measured using an Agilent 6545XT AdvanceBio LC/Q-TOF instrument. The system comprises a 1290 Infinity II UHPLC mated to a high-resolution, accurate mass quadrupole time-of-flight mass spectrometer (6545XT) equipped with a Dual Jet Stream electrospray ionization source. Affinity-purified antibodies were normalized to 1.0 mg/mL concentration in Dulbecco's PBS (pH 7.4, without Ca²⁺/Mg²⁺), no further sample manipulation was performed. Samples were stored in a refrigerated autosampler set to 6° C. Data were acquired using Agilent MassHunter Acquisition 10.1 (Build 10.1.48). For intact mass measurements, the instrument was operated in high resolution, extended mass range mode (2 GHz), with the Quad AMU manually set to 800, and all acquisition done in positive Ion Polarity. Instrument parameters were: Gas Temp=350° C., Gas Flow=12 L/min, Nebulizer=60 psig, Sheath Gas Temp=400° C., Sheath Gas Flow=11. Scan source parameters were: VCap=5500 V, Nozzle Voltage=2000 V, Fragmentor=380, Skimmer1=140, and OctopoleRFPeak=750. MS1 scans were acquired at a scan rate of 1.00 spectrum/s over a region of 850 to 5000 m/z. Reference mass correction was enabled using the m/z 922 mass calibrant. Samples were chromatographed using Agilent PLRP-S column (2.1 mm×50 mm, 5 μm, 1000 Å) heated to 60° C. in a column oven. Mobile phase A was 0.1% formic acid in water (v/v), mobile phase b was 99.9% acetonitrile with 0.1% formic acid (v/v) and the flow rate was 0.50 mL/min. The column was equilibrated in 20% B. Sample (typically 1 μg) was injected and briefly desalted on column for 2 min with the flow diverted to waste using the diverter valve on the Q-TOF (VCap and Nozzle Voltage values were both set to zero during this time segment). After 2 min, the diverter was switched to MS and gradient acquisition began. A steep gradient from 20% B to 100% was conducted over 3 min followed by a 1-min hold at 100% B, a return to 20% B over 1 min, and held for 1 min for equilibration. Samples were processed using Agilent MassHunter BioConfirm 10.0 (Build 10.1.10136.0). Spectra were averaged and deconvoluted using the Maximum Entropy algorithm to produce zero-charge spectra. Mass values of individual chains and post-translational modifications were calculated using the BioConfirm Chemical Dictionary and tables of proteoform masses were manually created and curated.

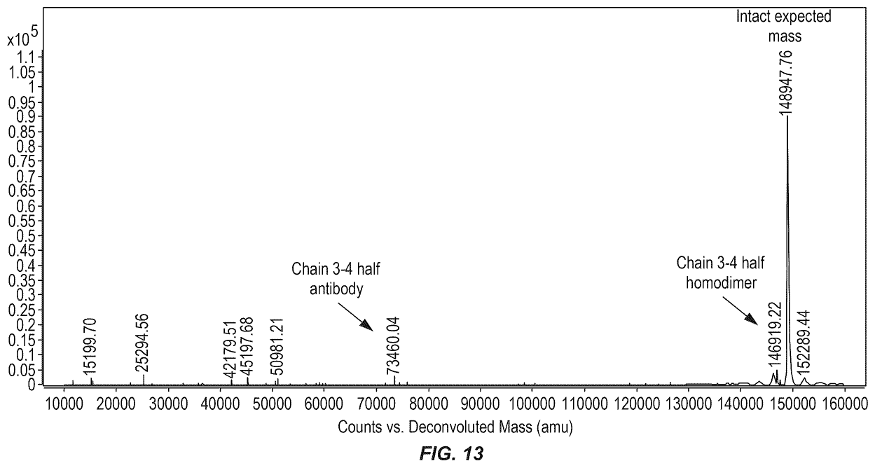

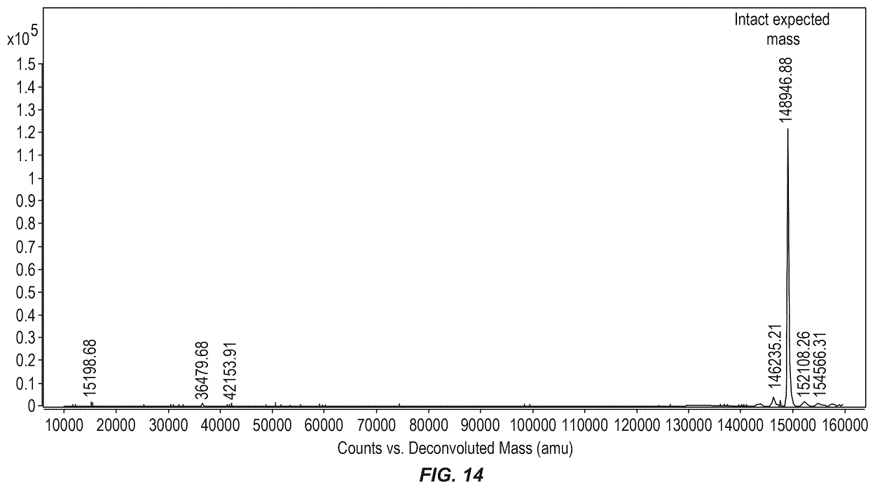

Exemplary results are shown in FIG. 13 and FIG. 14. FIG. 13 shows the LC-MS results for protein A isolated antibody, whereas as FIG. 14 shows the LC-MS results for protein A isolated antibody followed by purification with CEX. Results indicate protein A isolated antibody preparations contained both half antibodies (Half—3,4) and homodimers (Hole-Hole—3,4,3,4) as depicted in FIG. 6 as impurities, whereas the further purification with CEX removed these impurities.

### Example 7. Viscosity Analysis

Viscosity profiling for Exemplary Bispecific Antibody 1 (Ex. bsAb. 1) and an afucosylated version of Exemplary Bispecific Antibody 1 (Ex. bsAb. 1—afucos) was performed.

Viscosity is an important attribute for manufacturability and delivery/dose preparation. During the manufacturing process of a pharmaceutical composition comprising antibodies for administration by subcutaneous injection, the antibodies will often be concentrated and exchanged into a target buffer, which can include using ultrafiltration/diafiltration (UF/DF). The viscosity of a solution containing such antibodies is expected to increase during concentration. However, some solutions can experience sharp increases in viscosity at high concentrations of antibodies. The upper limit for most current UF/DF processing is approximately 80 cP. For delivery by subcutaneous injection, the viscosity should be about 15 cP or lower to enable ease of administration when pushing the plunger of a syringe containing the solution.

In order to assess viscosity, various concentrations of solutions containing either Ex. bsAb. 1 or Ex. bsAb. 1—afucos, and 10 mM histidine at pH 6.0 were generated. Each solution was assayed using a VROC (viscometer-rheometer-on-a-chip) initium one plus (RheoSense) at 25° C. measuring the viscosity as a function of the pressure drop, the dimensions of the microchannel, and the flow rate. Glycerol standards at 6 cP and 100 cP were used to calibrate the instrument and assess system suitability.

The viscosity profiling results are provided in Table 5. These results showed that both Ex. bsAb. 1 and Ex. bsAb. 1—afucos can be concentrated to approximately 170 mg/mL and still be less than or about equal to the preferred viscosity limit (15 cP) for subcutaneous administration. Moreover, these results showed that both Ex. bsAb. 1 and Ex. bsAb. 1—afucos can be concentrated to over 200 mg/mL and still be less than or approximately equal to the preferred viscosity limit (80 cP) for UF/DF.

TABLE 5
Viscosity Analysis

| Sample | Viscosity (cP) at 25° C. |
|---|---|
| 10 mM Histidine pH 6 Buffer | 0.9 |
| Ex. bsAb. 1: 47.4 mg/mL | 1.4 |
| Ex. bsAb. 1: 126.8 mg/mL | 4.8 |
| Ex. bsAb. 1: 157.4 mg/mL | 9.9 |
| Ex. bsAb. 1: 181.7 mg/mL | 17.4 |
| Ex. bsAb. 1: 227.1 mg/mL | 83.5 |
| Ex. bsAb. 1- afucos: 55.8 mg/mL | 1.7 |
| Ex. bsAb. 1- afucos: 137.6 mg/mL | 5.1 |
| Ex. bsAb. 1- afucos: 159.4 mg/mL | 11.4 |
| Ex. bsAb. 1- afucos: 185.7 mg/mL | 23.0 |
| Ex. bsAb. 1- afucos: 203.8 mg/mL | 48.8 |

### Example 8. Thermal Stability Measured by Scanning Calorimetry

Thermal stability for Exemplary Bispecific Antibody 1 (Ex. bsAb. 1) and an afucosylated version of Exemplary Bispecific Antibody 1 (Ex. bsAb. 1—afucos) was assessed.

The thermal stability of Ex. bsAb. 1 and Ex. bsAb. 1—afucos was determined by Differential Scanning Calorimetry (DSC). Briefly, protein solutions were diluted to ˜1 mg/mL in a reference buffer of 10 mM histidine, 8% (w/v) sucrose, and a pH 6.0, and were heated at 1° C./min from 20° C. to 100° C. The difference in heat absorbed between the solution and a reference was recorded. Melting point peaks in the resulting thermogram (e.g., Tm's or transition temperatures) represent the half-way point of unfolding of the domains of the proteins. Lower Tm values correspond to lower domain stability than higher Tm values. The profile of a standard IgG1 (NIST mAb) is also assessed.

The thermal stability results are provided in Table 6. Both Ex. bsAb. 1 and Ex. bsAb. 1—afucos exhibited similar thermograms indicating that both bsAb have comparable thermal stability. The profile of the standard IgG1 is also shown for reference. Both Ex. bsAb. 1 and Ex. bsAb. 1—afucos and the reference IgG1 show the same high onset, or start, of unfolding (˜58° C.), which show that Ex. bsAb. 1 and Ex. bsAb. 1—afucos have similar thermal stabilities as a standard IgG1.

TABLE 6
Thermal stability

| | Onset (° C.) | Tm1 | Tm2 | Tm3 |
|---|---|---|---|---|
| Molecule | of unfolding | (° C.) | (° C.) | (° C.) |
| Ex. bsAb. 1 | 58.0 | 69.0 | 77.4 | N/A |
| Ex. bsAb. 1 - afucos | 58.0 | 69.3 | 77.5 | N/A |
| NIST mAb (standard) | 58.0 | 70.0 | 83.5 | 93.2 |

### Example 9. Storage and Freeze-Thaw Stability

The storage stability was determined for Exemplary Bispecific Antibody 1 (Ex. bsAb. 1) and an afucosylated version of Exemplary Bispecific Antibody 1 (Ex. bsAb. 1—afucos). Briefly, samples at a concentration of 50 mg/mL were prepared in a buffer containing 10 mM histidine and 8% (w/v) sucrose at pH 6, and stored for 4 weeks at 5° C. (storage), 25° C. (accelerated), and 40° C. (stress). At each timepoint, samples were pulled and stored at −80° C., and later thawed and analyzed by SEC and MS.

SEC analysis showed an increase in high molecule weight species (HMWS) regardless of storage temperature, however the samples containing Ex. bsAb. 1—afucos showed a negligible increase (about 0.3%) at 5° C. MS analysis showed slight increases (<2.5%) in deamidation at heavy chain N33 and oxidation at light chain M105, regardless of storage temperatures. All samples at all conditions and timepoints were colorless, clear to slightly opalescent, and free of visible particles and precipitation.

The freeze-thaw stability of Ex. bsAb. 1 and Ex. bsAb. 1—afucos was also evaluated. Samples at a concentration of 50 mg/mL were prepared in a buffer containing 10 mM histidine and 8% (w/v) sucrose at pH 6. They were divided into three tubes. The first tube served as the TO condition without undergoing any freezing. The second and third tubes went through 2 and 4 freeze/thaw cycles, respectively. This process included freezing at −80° C. and subsequent thawing at 25° C. Samples were analyzed by SEC.

The results from SEC suggested that freeze-thawing had no impact on the molecular physical stability for both Ex. bsAb. 1 and Ex. bsAb. 1—afucos.

SEQUENCES
Exemplary Multispecific Binding Agents
Exemplary Bispecific Antibody 1 (B7H3 x GD2)
>First Polypeptide Chain of a B7H3 antibody construct (Domains A-B-D-E)
DIQMTQSPSSLSASVGDRVTITCRASQSVSSAVAWYQQKPGKAPKLLIYSASSLYS
GVPSRFSGSRSGTDFTLTISSLQPEDFATYYCQQSGRSLYTFGQGTKVEIKGQPRE
PQVCTLPPSRDELTKNQVSLTCLVRGFYPSDIAVEWESNGQPENNYKTTPPVLDSD
GSFFLYSKLTVDKSRWQQGNVFSCSVMHEALHNHYTQKSLSLSPGKDKTHTCPPC
PAPELLGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKTISKAKG
QPREPQVYTLPPSRDELTKNQVSLWCLVKGFYPSDIAVEWESNGQPENNYKTTPP
VLDSDGSFFLYSKLTVDKSRWQQGNVFSCSVMHEALHNHYTQKSLSLSPGK (SEQ
ID NO: 53)
>Second Polypeptide Chain of a B7H3 antibody construct (Domains F-G)
EVQLVESGGGLVQPGGSLRLSCAASGFTFSTYYIHWVRQAPGKGLEWVAWIHPSG
KYTYYADSVKGRFTISADTSKNTAYLQMNSLRAEDTAVYYCARHYQVGAMDYWG
QGTLVTVSSAQPREPQVYTLPPCRDWLTKNQVSLTCLVKGFYPSDIAVEWESNGQ
PENNYKTTPPVLDSDGSFFLYSKLTVDKSRWQQGNVFSCSVMHEALHNHYTQKSL
SLSPGK (SEQ ID NO: 54)
>Third Polypeptide Chain of a GD2 antibody construct (Domains H-I-J-K)
DIQMTQSPSSLSASVGDRVTITCRSSQSLVHRNGNTYLHWYQQKPGKAPKLLIHKV
SNRFSGVPSRFSGSGSGTDFTLTISSLQPEDFATYYCSQSTHVPPLTFGQGTKVEI
KRTVAAPSVFIFPPSDEQLKSGTASVVCLLNNFYPREAKVQWKVDNALQSGNSQE
SVTEQDSKDSTYSLSSTLTLSKADYEKHKVYACEVTHQGLSSPVTKSFNRGECDKT
HTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEK
TISKAKGQPREPQVYTLPPSREEMTKNQVSLSCAVKGFYPSDIAVEWESNGQPEN
NYKTTPPVLDSDGSFFLVSKLTVDKSRWQQGNVFSCSVMHEALHNHYTQKSLSLS
PGK (SEQ ID NO: 55)
>Fourth Polypeptide Chain of a GD2 antibody construct (Domains L-M)
EVQLVESGGGLVQPGGSLRLSCAASGSSFTGYNMNWVRQAPGKGLEWVAAIDPY
YGGTYYADSVKGRFTISADTSKNTAYLQMNSLRAEDTAVYYCASGMEYWGQGTLV
TVSSASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKKVEPKSC (SEQ
ID NO: 56)
Exemplary Bispecific Antibody 2 (GD2 x B7H3)
>First Polypeptide Chain of a GD2 antibody construct (Domains A-B-D-E)
DIQMTQSPSSLSASVGDRVTITCRSSQSLVHRNGNTYLHWYQQKPGKAPKLLIHKV
SNRFSGVPSRFSGSGSGTDFTLTISSLQPEDFATYYCSQSTHVPPLTFGQGTKVEI
KGQPREPQVCTLPPSRDELTKNQVSLTCLVRGFYPSDIAVEWESNGQPENNYKTT
PPVLDSDGSFFLYSKLTVDKSRWQQGNVFSCSVMHEALHNHYTQKSLSLSPGKDK
THTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIE
KTISKAKGQPREPQVYTLPPSRDELTKNQVSLWCLVKGFYPSDIAVEWESNGQPE
NNYKTTPPVLDSDGSFFLYSKLTVDKSRWQQGNVFSCSVMHEALHNHYTQKSLSL
SPGK (SEQ ID NO: 57)
>Second Polypeptide Chain of a GD2 antibody construct (Domains F-G)
EVQLVESGGGLVQPGGSLRLSCAASGSSFTGYNMNWVRQAPGKGLEWVAAIDPY
YGGTYYADSVKGRFTISADTSKNTAYLQMNSLRAEDTAVYYCASGMEYWGQGTLV
TVSSAQPREPQVYTLPPCRDWLTKNQVSLTCLVKGFYPSDIAVEWESNGQPENNY
KTTPPVLDSDGSFFLYSKLTVDKSRWQQGNVFSCSVMHEALHNHYTQKSLSLSPG
K (SEQ ID NO: 58)
>Third Polypeptide Chain of a B7H3 antibody construct (Domains H-I-J-K)
DIQMTQSPSSLSASVGDRVTITCRASQSVSSAVAWYQQKPGKAPKLLIYSASSLYS
GVPSRFSGSRSGTDFTLTISSLQPEDFATYYCQQSGRSLYTFGQGTKVEIKRTVAA
PSVFIFPPSDEQLKSGTASVVCLLNNFYPREAKVQWKVDNALQSGNSQESVTEQD
SKDSTYSLSSTLTLSKADYEKHKVYACEVTHQGLSSPVTKSFNRGECDKTHTCPPC
PAPELLGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKTISKAKG
QPREPQVYTLPPSREEMTKNQVSLSCAVKGFYPSDIAVEWESNGQPENNYKTTPP
VLDSDGSFFLVSKLTVDKSRWQQGNVFSCSVMHEALHNHYTQKSLSLSPGK (SEQ
ID NO: 59)
>Fourth Polypeptide Chain of a B7H3 antibody construct (Domains L-M)
EVQLVESGGGLVQPGGSLRLSCAASGFTFSTYYIHWVRQAPGKGLEWVAWIHPSG
KYTYYADSVKGRFTISADTSKNTAYLQMNSLRAEDTAVYYCARHYQVGAMDYWG
QGTLVTVSSASTKGPSVFPLAPSSKSTSGGTAALGCLVKDYFPEPVTVSWNSGALT
SGVHTFPAVLQSSGLYSLSSVVTVPSSSLGTQTYICNVNHKPSNTKVDKKVEPKSC
(SEQ ID NO: 60)
Exemplary Bispecific Antibody 3 (B7H3 x GD2; silent Fc)
>First Polypeptide Chain of a B7H3 antibody construct (Domains A-B-D-E)
DIQMTQSPSSLSASVGDRVTITCRASQSVSSAVAWYQQKPGKAPKLLIYSASSLYS
GVPSRFSGSRSGTDFTLTISSLQPEDFATYYCQQSGRSLYTFGQGTKVEIKGQPRE
PQVCTLPPSRDELTKNQVSLTCLVRGFYPSDIAVEWESNGQPENNYKTTPPVLDSD
GSFFLYSKLTVDKSRWQQGNVFSCSVMHEALHNHYTQKSLSLSPGKDKTHTCPPC
PAPEAAGGPSVFLFPPKPKDTLMISRTPEVTCVVVDVSHEDPEVKFNWYVDGVEV
HNAKTKPREEQYNSTYRVVSVLTVLHQDWLNGKEYKCKVSNKALKAPIEKTISKAK
GQPREPQVYTLPPSRDELTKNQVSLWCLVKGFYPSDIAVEWESNGQPENNYKTTP
PVLDSDGSFFLYSKLTVDKSRWQQGNVFSCSVMHEALHNHYTQKSLSLSPGK
(SEQ ID NO: 61)
>Second Polypeptide Chain of a B7H3 antibody construct (Domains F-G)
EVQLVESGGGLVQPGGSLRLSCAASGFTFSTYYIHWVRQAPGKGLEWVAWIHPSG
KYTYYADSVKGRFTISADTSKNTAYLQMNSLRAEDTAVYYCARHYQVGAMDYWG
QGTLVTVSSAQPREPQVYTLPPCRDWLTKNQVSLTCLVKGFYPSDIAVEWESNGQ
PENNYKTTPPVLDSDGSFFLYSKLTVDKSRWQQGNVFSCSVMHEALHNHYTQKSL
SLSPGK (SEQ ID NO: 62)
>Third Polypeptide Chain of a GD2 antibody construct (Domains H-I-J-K)
DIQMTQSPSSLSASVGDRVTITCRSSQSLVHRNGNTYLHWYQQKPGKAPKLLIHKV
SNRFSGVPSRFSGSGSGTDFTLTISSLQPEDFATYYCSQSTHVPPLTFGQGTKVEI
KRTVAAPSVFIFPPSDEQLKSGTASVVCLLNNFYPREAKVQWKVDNALQSGNSQE
SVTEQDSKDSTYSLSSTLTLSKADYEKHKVYACEVTHQGLSSPVTKSFNRGECDKT
HTCPPCPAPEAAGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALKAPIEK
TISKAKGQPREPQVYTLPPSREEMTKNQVSLSCAVKGFYPSDIAVEWESNGQPEN
NYKTTPPVLDSDGSFFLVSKLTVDKSRWQQGNVFSCSVMHEALHNHYTQKSLSLS
PGK (SEQ ID NO: 63)
>Fourth Polypeptide Chain of a GD2 antibody construct (Domains L-M)
EVQLVESGGGLVQPGGSLRLSCAASGSSFTGYNMNWVRQAPGKGLEWVAAIDPY
YGGTYYADSVKGRFTISADTSKNTAYLQMNSLRAEDTAVYYCASGMEYWGQGTLV
TVSSASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKKVEPKSC (SEQ
ID NO: 64)
Exemplary Bispecific Antibody 4 (GD2 x B7H3; silent Fc)
>First Polypeptide Chain of a GD2 antibody construct (Domains A-B-D-E)
DIQMTQSPSSLSASVGDRVTITCRSSQSLVHRNGNTYLHWYQQKPGKAPKLLIHKV
SNRFSGVPSRFSGSGSGTDFTLTISSLQPEDFATYYCSQSTHVPPLTFGQGTKVEI
KGQPREPQVCTLPPSRDELTKNQVSLTCLVRGFYPSDIAVEWESNGQPENNYKTT
PPVLDSDGSFFLYSKLTVDKSRWQQGNVFSCSVMHEALHNHYTQKSLSLSPGKDK
THTCPPCPAPEAAGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALKAPIE
KTISKAKGQPREPQVYTLPPSRDELTKNQVSLWCLVKGFYPSDIAVEWESNGQPE
NNYKTTPPVLDSDGSFFLYSKLTVDKSRWQQGNVFSCSVMHEALHNHYTQKSLSL
SPGK (SEQ ID NO: 65)
>Second Polypeptide Chain of a GD2 antibody construct (Domains F-G)
EVQLVESGGGLVQPGGSLRLSCAASGSSFTGYNMNWVRQAPGKGLEWVAAIDPY
YGGTYYADSVKGRFTISADTSKNTAYLQMNSLRAEDTAVYYCASGMEYWGQGTLV
TVSSAQPREPQVYTLPPCRDWLTKNQVSLTCLVKGFYPSDIAVEWESNGQPENNY
KTTPPVLDSDGSFFLYSKLTVDKSRWQQGNVFSCSVMHEALHNHYTQKSLSLSPG
K (SEQ ID NO: 66)
>Third Polypeptide Chain of a B7H3 antibody construct (Domains H-I-J-K)
DIQMTQSPSSLSASVGDRVTITCRASQSVSSAVAWYQQKPGKAPKLLIYSASSLYS
GVPSRFSGSRSGTDFTLTISSLQPEDFATYYCQQSGRSLYTFGQGTKVEIKRTVAA
PSVFIFPPSDEQLKSGTASVVCLLNNFYPREAKVQWKVDNALQSGNSQESVTEQD
SKDSTYSLSSTLTLSKADYEKHKVYACEVTHQGLSSPVTKSFNRGECDKTHTCPPC
PAPEAAGGPSVFLFPPKPKDTLMISRTPEVTCVVVDVSHEDPEVKFNWYVDGVEV
HNAKTKPREEQYNSTYRVVSVLTVLHQDWLNGKEYKCKVSNKALKAPIEKTISKAK
GQPREPQVYTLPPSREEMTKNQVSLSCAVKGFYPSDIAVEWESNGQPENNYKTTP
PVLDSDGSFFLVSKLTVDKSRWQQGNVFSCSVMHEALHNHYTQKSLSLSPGK
(SEQ ID NO: 67)
>Fourth Polypeptide Chain of a B7H3 antibody construct (Domains L-M)
EVQLVESGGGLVQPGGSLRLSCAASGFTFSTYYIHWVRQAPGKGLEWVAWIHPSG
KYTYYADSVKGRFTISADTSKNTAYLQMNSLRAEDTAVYYCARHYQVGAMDYWG
QGTLVTVSSASTKGPSVFPLAPSSKSTSGGTAALGCLVKDYFPEPVTVSWNSGALT
SGVHTFPAVLQSSGLYSLSSVVTVPSSSLGTQTYICNVNHKPSNTKVDKKVEPKSC
(SEQ ID NO: 68)
ADDITIONAL SEQUENCES
Antibody Architecture Constant Region(s) Sequences
(Identified alterations are according to the EU numbering system)
>First Polypeptide Chain Constant Regions
GQPREPQVCTLPPSRDELTKNQVSLTCLVRGFYPSDIAVEWESNGQPENNYKTTP
PVLDSDGSFFLYSKLTVDKSRWQQGNVFSCSVMHEALHNHYTQKSLSLSPGKDKT
HTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEK
TISKAKGQPREPQVYTLPPSRDELTKNQVSLWCLVKGFYPSDIAVEWESNGQPEN
NYKTTPPVLDSDGSFFLYSKLTVDKSRWQQGNVFSCSVMHEALHNHYTQKSLSLS
PGK (SEQ ID NO: 69)
>Second Polypeptide Chain Constant Region
AQPREPQVYTLPPCRDWLTKNQVSLTCLVKGFYPSDIAVEWESNGQPENNYKTTP
PVLDSDGSFFLYSKLTVDKSRWQQGNVFSCSVMHEALHNHYTQKSLSLSPGK
(SEQ ID NO: 70)
>Third Polypeptide Chain Constant Regions
RTVAAPSVFIFPPSDEQLKSGTASVVCLLNNFYPREAKVQWKVDNALQSGNSQES
VTEQDSKDSTYSLSSTLTLSKADYEKHKVYACEVTHQGLSSPVTKSFNRGECDKTH
TCPPCPAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTI
SKAKGQPREPQVYTLPPSREEMTKNQVSLSCAVKGFYPSDIAVEWESNGQPENNY
KTTPPVLDSDGSFFLVSKLTVDKSRWQQGNVFSCSVMHEALHNHYTQKSLSLSPG
K (SEQ ID NO: 71)
>Fourth Polypeptide Chain Constant Region
ASTKGPSVFPLAPSSKSTSGGTAALGCLVKDYFPEPVTVSWNSGALTSGVHTFPAV
LQSSGLYSLSSVVTVPSSSLGTQTYICNVNHKPSNTKVDKKVEPKSC (SEQ ID NO:
72)
>Domain B (CH3-Y349C; K370R)
GQPREPQVCTLPPSRDELTKNQVSLTCLVRGFYPSDIAVEWESNGQPENNYKTTP
PVLDSDGSFFLYSKLTVDKSRWQQGNVFSCSVMHEALHNHYTQKSLSLSPGK
(SEQ ID NO: 73)
>Domain D (CH2)
APELLGGPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKTISKAK
(SEQ ID NO: 74)
>Domain E (CH3-T366W)
GQPREPQVYTLPPSRDELTKNQVSLWCLVKGFYPSDIAVEWESNGQPENNYKTTP
PVLDSDGSFFLYSKLTVDKSRWQQGNVFSCSVMHEALHNHYTQKSLSLSPGK
(SEQ ID NO: 75)
>Domain G (CH3-G341A; S354C; E357W)
AQPREPQVYTLPPCRDWLTKNQVSLTCLVKGFYPSDIAVEWESNGQPENNYKTTP
PVLDSDGSFFLYSKLTVDKSRWQQGNVFSCSVMHEALHNHYTQKSLSLSPGK
(SEQ ID NO: 70)
>Domain I (CL-Kappa)
RTVAAPSVFIFPPSDEQLKSGTASVVCLLNNFYPREAKVQWKVDNALQSGNSQES
VTEQDSKDSTYSLSSTLTLSKADYEKHKVYACEVTHQGLSSPVTKSFNRGEC (SEQ
ID NO: 76)
>Domain J (CH2)
APELLGGPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKTISKAK
(SEQ ID NO: 77)
>Domain K (CH3-D356E, L358M, T366S, L368A, Y407V)
GQPREPQVYTLPPSREEMTKNQVSLSCAVKGFYPSDIAVEWESNGQPENNYKTTP
PVLDSDGSFFLVSKLTVDKSRWQQGNVFSCSVMHEALHNHYTQKSLSLSPGK
(SEQ ID NO: 78)
>Domain M (CH1)
ASTKGPSVFPLAPSSKSTSGGTAALGCLVKDYFPEPVTVSWNSGALTSGVHTFPAV
LQSSGLYSLSSVVTVPSSSLGTQTYICNVNHKPSNTKVDKKVEPKSC (SEQ ID NO:
72)

Throughout this application various publications, patents, patent applications and other documents have been referenced. The disclosures of these publications, patents, patent applications and other documents in their entireties are hereby incorporated by reference in this application for all purposes, including in order to more fully describe the state of the art to which this the subject matter disclosed herein pertains. Although the disclosed subject matter has been described with reference to the examples provided above, it should be understood that various modifications can be made without departing from the spirit of the disclosed subject matter. Many variations will become apparent to those skilled in the art upon review of this specification.

1. A bispecific antibody comprising a first polypeptide chain, a second polypeptide chain, a third polypeptide chain, and a fourth polypeptide chain, wherein the first polypeptide chain and the second polypeptide chain comprise an antigen binding domain for B7 Homolog 3 (B7H3), wherein the third polypeptide chain and the fourth polypeptide chain comprise an antigen binding domain for disialoganglioside (GD2), and wherein:
(A) the first polypeptide chain comprises the amino acid sequence of SEQ ID NO: 53;
(B) the second polypeptide chain comprises the amino acid sequence of SEQ ID NO: 54;
(C) the third polypeptide chain comprises the amino acid sequence of SEQ ID NO: 55;
(D) the fourth polypeptide chain comprises the amino acid sequence of SEQ ID NO: 56.

2. A bispecific antibody comprising a first polypeptide chain, a second polypeptide chain, a third polypeptide chain, and a fourth polypeptide chain, wherein the first polypeptide chain and the second polypeptide chain comprise an antigen binding domain for disialoganglioside (GD2), wherein the third polypeptide chain and the fourth polypeptide chain comprise an antigen binding domain for B7 Homolog 3 (B7H3), and wherein:
(A) the first polypeptide chain comprises the amino acid sequence of SEQ ID NO: 57;
(B) the second polypeptide chain comprises the amino acid sequence of SEQ ID NO: 58;
(C) the third polypeptide chain comprises the amino acid sequence of SEQ ID NO: 59;
(D) the fourth polypeptide chain comprises the amino acid sequence of SEQ ID NO: 60.

3. A bispecific antibody comprising a first polypeptide chain, a second polypeptide chain, a third polypeptide chain, and a fourth polypeptide chain, wherein the first polypeptide chain and the second polypeptide chain comprise an antigen binding domain for B7 Homolog 3 (B7H3), wherein the third polypeptide chain and the fourth polypeptide chain comprise an antigen binding domain for disialoganglioside (GD2), and wherein:
(A) the first polypeptide chain comprises a light chain variable (VL) region and constant regions, wherein the VL region comprises a VL CDR1, a VL CDR2, and a VL CDR3 amino acid sequence as set forth in SEQ ID NO: 26 and the constant regions comprise the amino acid sequence of SEQ ID NO: 69;
(B) the second polypeptide chain comprises a heavy chain variable (VH) region and a constant region, wherein the VH region comprises a VH CDR1, a VH CDR2, and a VH CDR3 amino acid sequence as set forth in SEQ ID NO: 25 and the constant region comprises the amino acid sequence of SEQ ID NO: 70;
(C) the third polypeptide chain comprises a light chain variable (VL) region and constant regions, wherein the VL region comprises a VL CDR1, a VL CDR2, and a VL CDR3 amino acid sequence as set forth in SEQ ID NO: 52 and the constant regions comprise the amino acid sequence of SEQ ID NO: 71;
(D) the fourth polypeptide chain comprises a heavy chain variable (VH) region and a constant region, wherein the VH region comprises a VH CDR1, a VH CDR2, and a VH CDR3 amino acid sequence as set forth in SEQ ID NO: 51 and the constant region comprises the amino acid sequence of SEQ ID NO: 72.

4-6. (canceled)

7. A bispecific antibody comprising a first polypeptide chain, a second polypeptide chain, a third polypeptide chain, and a fourth polypeptide chain, wherein the first polypeptide chain and the second polypeptide chain comprise an antigen binding domain for disialoganglioside (GD2), wherein the third polypeptide chain and the fourth polypeptide chain comprise an antigen binding domain for B7 Homolog 3 (B7H3), and wherein:
(A) the first polypeptide chain comprises a light chain variable (VL) region and constant regions, wherein the VL region comprises a VL CDR1, a VL CDR2, and a VL CDR3 amino acid sequence as set forth in SEQ ID NO: 52 and the constant regions comprise the amino acid sequence of SEQ ID NO: 69;
(B) the second polypeptide chain comprises a heavy chain variable (VH) region and a constant region, wherein the VH region comprises a VH CDR1, a VH CDR2, and a VH CDR3 amino acid sequence as set forth in SEQ ID NO: 51 and the constant region comprises the amino acid sequence of SEQ ID NO: 70;
(C) the third polypeptide chain comprises a light chain variable (VL) region and constant regions, wherein the VL region comprises a VL CDR1, a VL CDR2, and a VL CDR3 amino acid sequence as set forth in SEQ ID NO: 26 and the constant regions comprise the amino acid sequence of SEQ ID NO: 71;
(D) the fourth polypeptide chain comprises a heavy chain variable (VH) region and a constant region, wherein the VH region comprises a VH CDR1, a VH CDR2, and a VH CDR3 amino acid sequence as set forth in SEQ ID NO: 25 and the constant region comprises the amino acid sequence of SEQ ID NO: 72.

8-16. (canceled)

17. The bispecific antibody of claim 1, wherein bispecific antibody is conjugated or recombinantly fused to a diagnostic agent, detectable agent, or therapeutic agent.

18. The bispecific antibody of claim 17, wherein the therapeutic agent is a chemotherapeutic agent, cytotoxin, or drug.

19. One or more vectors comprising one or more polynucleotides encoding the first polypeptide chain, the second polypeptide chain, the third polypeptide chain, and the fourth polypeptide chain of bispecific antibody of claim 1.

20. (canceled)

21. A method of generating a bispecific antibody composition comprising culturing a cell comprising the one or more vectors of claim 19 in a culture medium to produce the bispecific antibody in the culture medium, isolating the bispecific antibody from the culture medium, and purifying the bispecific antibody isolated from the culture medium, thereby generating the bispecific antibody composition.

22-23. (canceled)

24. A pharmaceutical composition comprising an effective amount of the bispecific antibody of claim 1 and a pharmaceutically acceptable carrier.

25-31. (canceled)

32. A method of treating or preventing a cancer in a subject comprising administering to the subject the bispecific antibody of claim 1.

33. A method for alleviating one or more symptoms associated with a cancer in a subject comprising administering to the subject the bispecific antibody of claim 1.

34-40. (canceled)

41. A method of selectively binding a cell co-expressing B7 Homolog 3 (B7H3) and disialoganglioside (GD2) in a subject comprising administering to the subject the bispecific antibody of claim 1.

42. The bispecific antibody of claim 2, wherein bispecific antibody is conjugated or recombinantly fused to a diagnostic agent, detectable agent, or therapeutic agent.

43. The bispecific antibody of claim 42, wherein the therapeutic agent is a chemotherapeutic agent, cytotoxin, or drug.

44. One or more vectors comprising one or more polynucleotides encoding the first polypeptide chain, the second polypeptide chain, the third polypeptide chain, and the fourth polypeptide chain of bispecific antibody of claim 2.

45. A method of generating a bispecific antibody composition comprising culturing a cell comprising the one or more vectors of claim 44 in a culture medium to produce the bispecific antibody in the culture medium, isolating the bispecific antibody from the culture medium, and purifying the bispecific antibody isolated from the culture medium, thereby generating the bispecific antibody composition.

46. A pharmaceutical composition comprising an effective amount of the bispecific antibody of claim 2 and a pharmaceutically acceptable carrier.

47. A method of treating or preventing a cancer in a subject comprising administering to the subject the bispecific antibody of claim 2.

48. A method for alleviating one or more symptoms associated with a cancer in a subject comprising administering to the subject the bispecific antibody of claim 2.

49. A method of selectively binding a cell co-expressing B7 Homolog 3 (B7H3) and disialoganglioside (GD2) in a subject comprising administering to the subject the bispecific antibody of claim 2.
